SEQUENCE LISTING (99 sequences)

SEQ ID NO: 1 - RNA, 19 nt, Artificial
  SMS2-i6 sense strand of duplex portion
gcauuuucuguaucagaaa

SEQ ID NO: 2 - RNA, 19 nt, Artificial
  SMS2-i6 antisense strand of duplex portion
uuucugauacagaaaaugc

SEQ ID NO: 3 - RNA, 19 nt, Artificial
  SMS2-i7 sense strand of duplex portion
gucacuucuggugguauca

SEQ ID NO: 4 - RNA, 19 nt, Artificial
  SMS2-i7 antisense strand of duplex portion
ugauaccaccagaagugac

SEQ ID NO: 5 - RNA, 19 nt, Artificial
  SMS2-i8 sense strand of duplex portion
cuguuuuggugguaccauu

SEQ ID NO: 6 - RNA, 19 nt, Artificial
  SMS2-i8 antisense strand of duplex portion
aaugguaccaccaaaacag

SEQ ID NO: 7 - RNA, 19 nt, Artificial
  SMS2-i104 sense strand of duplex portion
gggcauugccuucauauau

SEQ ID NO: 8 - RNA, 19 nt, Artificial
  SMS2-i104 antisense strand of duplex portion
auauaugaaggcaaugccc

SEQ ID NO: 9 - RNA, 19 nt, Artificial
  SMS2-i105 sense strand of duplex portion
ggcuguuucugagauacaa

SEQ ID NO: 10 - RNA, 19 nt, Artificial
  SMS2-i105 antisense strand of duplex portion
uuguaucucagaaacagcc

SEQ ID NO: 11 - RNA, 19 nt, Artificial
  SMS2-i106 sense strand of duplex portion
ggugguggauuguccauaa

SEQ ID NO: 12 - RNA, 19 nt, Artificial
  SMS2-i106 antisense strand of duplex portion
uuauggacaauccaccacc

SEQ ID NO: 13 - RNA, 19 nt, Artificial
  SMS2-i107 sense strand of duplex portion
ggauuguccauaacuggau

SEQ ID NO: 14 - RNA, 19 nt, Artificial
  SMS2-i107 antisense strand of duplex portion
auccaguuauggacaaucc

SEQ ID NO: 15 - RNA, 19 nt, Artificial
  SMS2-i108 sense strand of duplex portion
ccauaacuggaucacauau

SEQ ID NO: 16 - RNA, 19 nt, Artificial
  SMS2-i108 antisense strand of duplex portion
auaugugauccaguuaugg

SEQ ID NO: 17 - RNA, 19 nt, Artificial
  SMS2-i109 sense strand of duplex portion
gcacacgaacacuacacua

SEQ ID NO: 18 - RNA, 19 nt, Artificial
  SMS2-i109 antisense strand of duplex portion
uaguguaguguucgugugc

SEQ ID NO: 19 - RNA, 19 nt, Artificial
  SMS2-i3 sense strand of duplex portion
ggaugguauugguuggguu

SEQ ID NO: 20 - RNA, 19 nt, Artificial
  SMS2-i3 antisense strand of duplex portion
aacccaaccaauaccaucc

SEQ ID NO: 21 - RNA, 19 nt, Artificial
  SMS2-i11 sense strand of duplex portion
ggcucuuucugcguuacaa

SEQ ID NO: 22 - RNA, 19 nt, Artificial
  SMS2-i11 antisense strand of duplex portion
uuguaacgcagaaagagcc

SEQ ID NO: 23 - RNA, 19 nt, Artificial
  SMS2-i1 sense strand of duplex portion
ggucacuuggaaagucaaa

SEQ ID NO: 24 - RNA, 19 nt, Artificial
  SMS2-i1 antisense strand of duplex portion
uuugacuuuccaagugacc

SEQ ID NO: 25 - RNA, 19 nt, Artificial
  SMS2-i2 sense strand of duplex portion
ccggacuacauccagauuu

SEQ ID NO: 26 - RNA, 19 nt, Artificial
  SMS2-i2 antisense strand of duplex portion
aaaucuggauguaguccgg

SEQ ID NO: 27 - RNA, 19 nt, Artificial
  SMS2-i5 sense strand of duplex portion
cauagagacagcaaaacuu

SEQ ID NO: 28 - RNA, 19 nt, Artificial
  SMS2-i5 antisense strand of duplex portion
aaguuuugcugucucuaug

SEQ ID NO: 29 - DNA, 13 bp, Artificial
  SMS2-13-003
tgataccaccaga

SEQ ID NO: 30 - DNA, 13 bp, Artificial
  SMS2-13-006
tgcagatgatccc

SEQ ID NO: 31 - DNA, 13 bp, Artificial
  SMS2-13-007
cgtgttgtgatat

SEQ ID NO: 32 - DNA, 13 bp, Artificial
  SMS2-13-008
acttgtctgggag

SEQ ID NO: 33 - DNA, 13 bp, Artificial
  SMS2-13-009
agaggaagtctcc

SEQ ID NO: 34 - DNA, 13 bp, Artificial
  SMS2-13-010
agatggggaacca

SEQ ID NO: 35 - DNA, 13 bp, Artificial
  SMS2-13-011
agtctccattgag

SEQ ID NO: 36 - DNA, 13 bp, Artificial
  SMS2-13-012
ccagaagtgacga

SEQ ID NO: 37 - DNA, 13 bp, Artificial
  SMS2-13-014
ttgcctgagagtc

SEQ ID NO: 38 - DNA, 13 bp, Artificial
  SMS2-13-017
aagttttgctgtc

SEQ ID NO: 39 - DNA, 13 bp, Artificial
  SMS2-13-019
ttgaagcagccag

SEQ ID NO: 40 - DNA, 13 bp, Artificial
  SMS2-13-020
gcagcaaggaatt

SEQ ID NO: 41 - RNA, 25 nt, Artificial
  SMS1 sense siRNA sequence
auacauuguaauacaccgauacagg

SEQ ID NO: 42 - RNA, 25 nt, Artificial
  SMS1 antisense siRNA sequence
ccuguaucgguguauuacaauguau

SEQ ID NO: 43 - RNA, 25 nt, Artificial
  SMS2 sense siRNA sequence
auacauaguuauacagcgauacagg

SEQ ID NO: 44 - RNA, 25 nt, Artificial
  SMS2 antisense siRNA sequence
ccuguaucgcuguauaacuauguau

SEQ ID NO: 45 - RNA, 25 nt, Artificial
  aSMase sense siRNA sequence
auugguuucccuuuaugaagggagg

SEQ ID NO: 46 - RNA, 25 nt, Artificial
  aSMase antisense siRNA sequence
ccucccuucauaaagggaaaccaau

SEQ ID NO: 47 - RNA, 25 nt, Artificial
  nSMase1 sense siRNA sequence
aauagaaccacaucugcauucuugg

SEQ ID NO: 48 - RNA, 25 nt, Artificial
  nSMase1 antisense siRNA sequence
ccaagaaugcagaugugguucuauu

SEQ ID NO: 49 - RNA, 25 nt, Artificial
  nSMase2 sense siRNA sequence
aaucgauguagaucuugaucugagg

SEQ ID NO: 50 - RNA, 25 nt, Artificial
  nSMase2 antisense siRNA sequence
ccucagaucaagaucuacaucgauu

SEQ ID NO: 51 - DNA, 16 bp, Artificial
  sense primer for human APP770
atgctgcccggtttgg

SEQ ID NO: 52 - DNA, 28 bp, Artificial
  antisense primer for human APP770
ctagttctgcatctgctcaaagaacttg

SEQ ID NO: 53 - DNA, 21 bp, Artificial
  SMS2-i1 sense strand
ggucacuuggaaagucaaann

SEQ ID NO: 54 - DNA, 21 bp, Artificial
  SMS2-i1 antisense strand
uuugacuuuccaagugaccnn

SEQ ID NO: 55 - DNA, 21 bp, Artificial
  SMS2-i2 sense strand
ccggacuacauccagauuunn

SEQ ID NO: 56 - DNA, 21 bp, Artificial
  SMS2-i2 antisense strand
aaaucuggauguaguccggnn

SEQ ID NO: 57 - DNA, 21 bp, Artificial
  SMS2-i3 sense strand
ggaugguauugguuggguunn

SEQ ID NO: 58 - DNA, 21 bp, Artificial
  SMS2-i3 antisense strand
aacccaaccaauaccauccnn

SEQ ID NO: 59 - DNA, 21 bp, Artificial
  SMS2-i4 sense strand
gcagauuguuguugaucaunn

SEQ ID NO: 60 - DNA, 21 bp, Artificial
  SMS2-i4 antisense strand
augaucaacaacaaucugcnn

SEQ ID NO: 61 - DNA, 21 bp, Artificial
  SMS2-i5 sense strand
cauagagacagcaaaacuunn

SEQ ID NO: 62 - DNA, 21 bp, Artificial
  SMS2-i5 antisense strand
aaguuuugcugucucuaugnn

SEQ ID NO: 63 - DNA, 21 bp, Artificial
  SMS2-i6 sense strand
gcauuuucuguaucagaaann

SEQ ID NO: 64 - DNA, 21 bp, Artificial
  SMS2-i6 antisense strand
uuucugauacagaaaaugcnn

SEQ ID NO: 65 - DNA, 21 bp, Artificial
  SMS2-i7 sense strand
gucacuucuggugguaucann

SEQ ID NO: 66 - DNA, 21 bp, Artificial
  SMS2-i7 antisense strand
ugauaccaccagaagugacnn

SEQ ID NO: 67 - DNA, 21 bp, Artificial
  SMS2-i8 sense strand
cuguuuuggugguaccauunn

SEQ ID NO: 68 - DNA, 21 bp, Artificial
  SMS2-i8 antisense strand
aaugguaccaccaaaacagnn

SEQ ID NO: 69 - DNA, 21 bp, Artificial
  SMS2-i11 sense strand
ggcucuuucugcguuacaann

SEQ ID NO: 70 - DNA, 21 bp, Artificial
  SMS2-i11 antisense strand
uuguaacgcagaaagagccnn

SEQ ID NO: 71 - DNA, 21 bp, Artificial
  SMS2-i104 sense strand
gggcauugccuucauauaunn

SEQ ID NO: 72 - DNA, 21 bp, Artificial
  SMS2-i104 antisense strand
auauaugaaggcaaugcccnn

SEQ ID NO: 73 - DNA, 21 bp, Artificial
  SMS2-i105 sense strand
ggcuguuucugagauacaann

SEQ ID NO: 74 - DNA, 21 bp, Artificial
  SMS2-i105 antisense strand
uuguaucucagaaacagccnn

SEQ ID NO: 75 - DNA, 21 bp, Artificial
  SMS2-i106 sense strand
ggugguggauuguccauaann

SEQ ID NO: 76 - DNA, 21 bp, Artificial
  SMS2-i106 antisense strand
uuauggacaauccaccaccnn

SEQ ID NO: 77 - DNA, 21 bp, Artificial
  SMS2-i107 sense strand
ggauuguccauaacuggaunn

SEQ ID NO: 78 - DNA, 21 bp, Artificial
  SMS2-i107 antisense strand
auccaguuauggacaauccnn

SEQ ID NO: 79 - DNA, 21 bp, Artificial
  SMS2-i108 sense strand
ccauaacuggaucacauaunn

SEQ ID NO: 80 - DNA, 21 bp, Artificial
  SMS2-i108 antisense strand
auaugugauccaguuauggnn

SEQ ID NO: 81 - DNA, 21 bp, Artificial
  SMS2-i109 sense strand
gcacacgaacacuacacuann

SEQ ID NO: 82 - DNA, 21 bp, Artificial
  SMS2-i109 antisense strand
uaguguaguguucgugugcnn

SEQ ID NO: 83 - DNA, 5269 bp, Homo sapiens
  CDS (423)..(2390)
gctgagtctgagggaggctccggacccgagagccgcgagagccgccgccgctgcggccgccgccagatctgcggccgggagcccgggctgtgaggagccgggaggagcggggtgcgctgccgggcgctgaccgccctcccgcccgccgtcagaggtctgcggtgacagctcttcttcagagagaaggacaacaaggtcccagtggcccctcctcagggtctgcagtaggcctccgcatggcccaccgaggtgaaccatgaccggctggccaacattcgccattgaccagccggagttgcatctcgccaggaggtgacccctcctccagctgcccccaactcgcccaccctcgcccaggaaagtgcccgcagctgccacggacaccatgtagtagggccggctgcggcgcccagtgagctgcgatggttttgtacacgaccccctttcctaacagctgtctgtccgccctgcactgtgtgtcctgggcccttatctttccatgctactggctggtggaccggctcgctgcctccttcatacccaccacctacgagaagcgccagcgggcagacgacccgtgctgcctgcagctgctctgcactgccctcttcacgcccatctacctggccctcctggtggcctcgctgccctttgcgtttctcggctttctcttctggtccccactgcagtcggcccgccggccctacatctattcacggctggaagacaagggcctggccggtggggcagccctgctcagtgaatggaagggcacggggcctggcaaaagcttctgctttgccactgccaacgtctgcctcctgcccgactcactcgccagggtcaacaacctttttaacacccaagcgcgggccaaggagatcgggcagagaatccgcaatggggccgcccggccccagatcaaaatttacatcgactcccccaccaatacctccatcagcgccgctagcttcagcagcctggtgtcaccacagggcggcgatggggtggcccgggccgtccccgggagcattaagaggacagcctctgtggagtacaagggtgacggtgggcggcaccccggtgacgaggctgccaacggcccagcctctggggaccctgtcgacagcagcagcccggaggatgcctgcatcgtgcgcatcggtggcgaggagggcggccggccacctgaagctgacgaccctgtgcctgggggccaggccaggaacggagctggcgggggcccaaggggccagacgcccaaccataatcagcaggacggggattcagggagcctgggcagcccctcggcctcccgggagtccctggtgaaggggcgagctgggccagacaccagtgccagcggggagccaggtgccaacagcaagctcctgtacaaggcctcggtggtgaagaaggcggctgcacgcaggaggcggcaccccgacgaggccttcgaccatgaggtctccgccttcttccccgccaacctggacttcctgtgcctgcaggaggtgtttgacaagcgagcagccaccaaattgaaagagcagctgcacggctacttcgagtacatcctgtacgacgtcggggtctacggctgccagggctgctgcagcttcaagtgtctcaacagcggcctcctctttgccagccgctaccccatcatggacgtggcctatcactgttaccccaacaagtgtaacgacgatgccctggcctctaagggagctctgtttctcaaggtgcaggtgggaagcacacctcaggaccaaagaatcgtcgggtacatcgcctgcacacacctgcatgccccgcaagaggacagcgccatccggtgtgggcagctggacctgcttcaggactggctggctgatttccgaaaatctacctcctcgtccagcgcagccaaccccgaggagctggtggcatttgacgtcgtctgtggagatttcaactttgataactgctcctctgacgacaagctggagcagcaacactccctgttcacccactacagggacccctgccgcctggggcctggtgaggagaagccgtgggccatcggtactctgctggacacgaacggcctgtacgatgaggatgtgtgcacccccgacaacctgcagaaggtcctggagagtgaggagggccgcagggagtacctggcgtttcccaccagcaagagctcgggccagaaggggcggaaggagctgctgaagggcaacggccggcgcatcgactacatgctgcatgcagaggaggggctgtgcccagactggaaggccgaggtggaagaattcagttttatcacccagctgtccggcctgacggaccacctgccagtagccatgcgactgatggtgtcttcgggggaggaggaggcatagaccgtccggagcagcggggcctctgccagcccttgcagctgcagcccatccctgggccatgtcccctccatcgagtgcccggtgcttgggggaggagggcagggacagggagggagccacagtcagtgcccgggaacctggaagctgcgctgctctgcgcctctgggcctcactgtggacagaggagtcaggcccgccccaggagcctccagctgcctaaccagtgccattctttcacaacacgattttctacaaatctacagcacaaccgagtttgtaacccgtgggttagtatgaggaccgggttcgtgtactctctgtatctcctcttaagcttcgtccagggttctttatttttgtctgctgccaatgtcgtctcgcatgcctgcaccctcgcatgcacgctgcccgcatgccacgtgccacgctgtagccacagaccccttgctcgggcctcacccaaggccaaactccaaacacaatcagaaccagccaaagaagcacttcctgggcacggccaccagctctcccgcctccagtgtgggccggctcctgcagggtccgagggctgcatctctaccagccagcccagggctcttcccagggtctcgcattcaagggcaattacattttaaaaagaaaaacagaaaaaggttaatcacaaaaccaaccctcacttcacagggtctgtaagtcactcatagaactttgctcttcccgagacagggtcccttccccagctcaggcacaacagagtctggcaggctctggcaccctgggcctcctccgggagcctcccatctgggcagtggagccataaacggggatccgagaagagagtatccacttttttttttacaggaagaagggactcacagcataaacgggggtgggggggatcctgattttgaaaataatctatttgtagcttctcttctatcaaaaccaacacatcctcttctttctgccaatcctctcccccacgggacacctctctggttcgggaccaatccctccctggggacgtgccccacctgcgtgccggctgagctcaggaacccctgcctgccccccgggtggggctgcggctctggcctcccaggcccatcctcaacagctaccccagccaacaccaaggccacaaggggaccccggcctaggaggcaggaagccaaggtgcagagagcagcctggccctcaccagtgcgcaagctggggcagcaaggctgacagttgctgcatgcccagggcagggtgtggtactggcacccaagttcagcatggcagagctggccaacagcttgtggtccccgatctgcctccagccccaagatgcctacagcccccaggccccttcggcagcactgcctctgcccacctgcctttaagagactccagggctgctcctgtcatgcagcgaaggttttgtctgtttcaaagttcgagactcaacttgagggactgtttttgacaatccccgctgacctccgctcctcgtggcgccctggccctacacccagcctggcccagggccggctttgcctggtgaggctggagggagcaccaggacctgctgtctgctgtcagcccctcctggtgctggtgccctgatgctgtgccttgtcacccattgagctgcaagagggaccaagagggggccacgcagccagccagatgcctggccctgtgctggggcagacaacgctgcagagcccagggagcctggcgctaggacgtgcgtccttgtgacactggcctgtctgaactcacctggcctgggaagcaccgtctgcccgggcccaagccctgcccctccagagtccagagccaggaaggggctgctgagggcgagcatcctgctgggctctctgcccggcccacccctccaaggggctggcctgtgagccttgactgggattcatgatgtggaggcccccaacttccagaagcagctggtactctgctcacacaagcgactgggccggccggccctggacccctagaccccgagccgcctgccgactgcctgcacagggagagcagttgaggcccgggcagggcccccacaccagaccccaacatagcttccccacccaggcaccccctcccggggcagcaggcgtgggagtcagggctgcatgctcctcccctcccacctcacaggcggccttaggcaagtcattttctgtcatcacaaggtcgcctctgcctagtcaggtcctggggtccagagtaaggatgtgcggcccccaggcccccgcacacctccctcagcaccaagaccgggacccccccacccacgtgtctcattgtggctgcctatggactcccgggccttgtgtgcaggccaggcccttccactgattttttaaagtgaaccattgctggatctcagattctgtggcatctaaggcctagcaggggtgggcacacgggtcacccgaggcccataccaagactctgttcctgccctaggcccagtctcaaaggaagccacaaggcgcgggggccactgaggaaggaaatgttcattttcatttgtccaaaaccaccttaagttttaagtatattaatcttgatgctttttaactattgctttttaacttgctgagatttagaaatactgttataaaaacttttttaatttctgtatttttttctgtattgtatcttcatgggacattaggggttttctatggtaagcacacctatggttttggtaaaaacattatcaaatatatatccagacggttcttccctagaagaaaaacaagtctttacacctgataaaatattttgcgaagagaggtgttctttttccttactggtgctgaaaggaaggatggataacgaggagaaaataaaactgtgaggctcaa

SEQ ID NO: 84 - protein, 655 aa, Homo sapiens
MVLYTTPFPNSCLSALHCVSWALIFPCYWLVDRLAASFIPTTYEKRQRADDPCCLQLLCTALFTPIYLALLVASLPFAFLGFLFWSPLQSARRPYIYSRLEDKGLAGGAALLSEWKGTGPGKSFCFATANVCLLPDSLARVNNLFNTQARAKEIGQRIRNGAARPQIKIYIDSPTNTSISAASFSSLVSPQGGDGVARAVPGSIKRTASVEYKGDGGRHPGDEAANGPASGDPVDSSSPEDACIVRIGGEEGGRPPEADDPVPGGQARNGAGGGPRGQTPNHNQQDGDSGSLGSPSASRESLVKGRAGPDTSASGEPGANSKLLYKASVVKKAAARRRRHPDEAFDHEVSAFFPANLDFLCLQEVFDKRAATKLKEQLHGYFEYILYDVGVYGCQGCCSFKCLNSGLLFASRYPIMDVAYHCYPNKCNDDALASKGALFLKVQVGSTPQDQRIVGYIACTHLHAPQEDSAIRCGQLDLLQDWLADFRKSTSSSSAANPEELVAFDVVCGDFNFDNCSSDDKLEQQHSLFTHYRDPCRLGPGEEKPWAIGTLLDTNGLYDEDVCTPDNLQKVLESEEGRREYLAFPTSKSSGQKGRKELLKGNGRRIDYMLHAEEGLCPDWKAEVEEFSFITQLSGLTDHLPVAMRLMVSSGEEEA

SEQ ID NO: 85 - DNA, 5148 bp, Mus musculus
  CDS (470)..(2437)
cgcggagctgttcgccgctccaggtgctgagtccgagggaggctccggatgccagagccgcggtcgctgtaaccatcgccgccgcgagagccgtctctaggaagccccggtcgcgaggagctgggaggcgccgcgtgcgctgccccgcaccgagcgccctcctgtgcgccacctgagaaaagaaaagaagagaagagaagagaagagaagagaagagaagagaagagaagagaagagaagagaaaaaaacacaacagttaaaaggttttcagcagactccggtgtcccagcatggccccttgaagcgaaccatgactgtctgtgggacactcgccactgatcatccagagtcacatctcaccaggaggtgacctctcctccatcctccacctgccccctgcctgggacagtgctgcggctgccacggacaccatgtagtagagccggctgcggcgcccagtgagctgcaatggttttgtacacgaccccctttcctaatagctgtctgtctgccctccacgccgtgtcctgggccctcatcttcccatgttactggctggtggaccggctgttggcctctttcatacccaccacctatgagaagcgccagagggcagatgacccctgctgcctgcagctgttctgcacggtcctcttcacgccagtctacctggctctgcttgtggctgcactgccctttgcctttctcgggttcatcttctggtctccactgcagtctgcccgccggccctactcctactcccggctagaagacaagaacccagctggtggggcagccctgcttagtgaatggaagggcacgggggcaggcaaaagcttttgcttcgccacggccaatgtctgcctcctccccgactcactcgcgaggctcaataatgtgttcaacactcaagctcgggccaaagagattgggcagagaatccgcaatggggccgccaggcctcagatcaagatctacatcgattctcccaccaacacctccatcagtgcagccagcttcagcagcctggtgtcgccacagggtggtgatggatccagagctgttcctgggagcattaagaggacagcctctgtggaatacaagggggatggtgggcgtcaccccagcgacgaggctgccaacggcccagcctctggagaacaggctgacggaagccttgaggacagctgcattgtgcgaattggtggcgaggaaggaggtcggccacaagaagctgatgaccctgctgccgggagccaagccaggaatggggctggtgggaccccaaagggccagacacccaaccacaatcagcgagatggggattctgggagcctgggcagcccctcagcctccagggagtccttggtgaaggcacgggctgggcaagacagtggtggcagcggagagccaggggctaacagcaagctgctgtataagacctctgtggtgaagaaggcagcagcccgcaggaggcgccaccctgatgaagcctttgaccacgaggtctcagccttcttcccggccaatctggacttcctgtgcctgcaggaggtgtttgacaagcgtgcggctgctaagttgaaagagcagctacacggctacttcgagtacatcctgtatgatgttggggtctacggttgtcatggttgctgcaatttcaaatgtctcaacagcggtctcttcttcgccagccgctaccctgtcatggacgtggcctatcactgttaccccaatgggtgcagcttcgatgccctggcctctaagggagctctgtttctcaaggtgcaggtgggaagcacacctcaggaccaaagaattgttgggtacatcgcctgcacacacctgcatgccccaccagaggacagtgccgtccggtgtgagcagctggacctgcttcaggactggctggctgatttccgaaaatctacctcctcgaccagcacagccaaccccgaggagctggtggtgtttgacgtcatctgtggagatttgaactttgataactgctcttctgatgacaaactggagcagcagcactcgctctttactcgctacaaagacccctgccgccttgggcccggggaggagaagccttgggccattggcaccctgctggacacaaacggtctctatgatgaggatgtgtgtaccccggacaaccttcaaaaggtgttggagagcgaggaaggccgcagggagtatctggccttccccaccagcaagagcccaggtgctgggcagaagggccggaaggatctgctgaagggcaatggccgccgcatcgactacatgctgcacgccgaggaggggctgtgccctgactggaaggctgaggtagaagaattcagttttatcacccagctgtccggcctgaccgaccacctccccgtggccatgcggctgatggtgtctgcaggggaagaggaggcatagaccagccatgccacggtctcgagcagtagggcctctgccggcccttcaagccacagcccgtgcctgggctgtgtgccctccattgagcacccagtgctgggggaaggagggcaggggacagggaaggacgagcccatcaatcccctgcaagcctgggaaccaggctgctctgtgcctctgggccaactctgcggacagaggacttgggccagcctgggagcccccggctgcctgaccagtgccattctttcagtcacgattttctacagaaatacacagcacaacctagtttgtaacccaagggttagtacgaggaccgggtttctgtactctttgtatctcttctcaagctttgtccagggttcattacttgtgtctgctgccaatgttgtctcgcatgcctgcaccctcgcatgcccactgcccgcatgccatgtgccacactgtagcacccacaccccttgctcgggcctcaaaggccaaactcaaaacacaaaaaacacaatcagaaccagccaaagaagcacttcctgggcacagctgccagctctcccccctccagcctggtggggagtgattgacggctgtcattacctgccagcccagggctcttcccagggtcttgcattcaagggcgattacattttaagaagaaaacagaaaaaaatgttacacacaaaatcacccttcccttagggtctgtaagtcactgggtagggctgtgctctttcccagcccccacagcagggtccagctggctctggccaccccagcagctcctataggagactcataccagctctggggccctaacaggaaatccaagagggcatcacttttgtacacgaaggggacccagaatataagggggtgggggttctgattttgaaactgacaggttcgtagctccttctctcctacccaacatgtcctcttctgtggggccctttgggttagcctcttcctgaggacttccctcatccatgtatggctgagctcaggaaccacttgcctgcccccaagtcaggctgcgcctctggcctcccaggcccacccttccacaatggctaccctggataaagtggcaggaaaccaagatagaatggcttctttaccactgcaagctcaggtggccagccaacagccactgcttgtctagagcagggctgactccagacagcatccagaagccactcttgccaacgtgtgactagagccagtcccaagctcagcacagtaaggctgctagaagcccactgctctccacctggtcctgctcaggtgcccaagagcactgatcctgccgacctcccttcagagaacggaggctgatccctgaccacacaggaagggacgacgtctgcctcaggctgctcagctactgatttgagggactatttgggtattcccaacggtctcttcagagtgctctggtcttgtatctggcctggcgatgatggagggagcatcaggcccctgtggttgggacctctaaaatgttggtgccctgaagctgtgccttgtcacccaccaagtagcaagagggatccagaggggcacacatttgtcagacgccaggctgtgctgagcagtgaccttcagagtatgggcagtttagtattagggcatggatagctggagacactggtctcttaacctcttggcctagagaaccctgcccatgctccagtgtccagagctaggacagccttgctgggttctctgctgtgcccctcttctgccagggcctggcctcccaggaatctgggaagccctcatggagtaaaaggcccctaactcctggaaacaaccaggatcctatccattcaaggtaccatggggcaggtccaatcttcaaaccaatggcactgtgcacaaggagagtagaggaggccgaggccaccgtcagggtccctaaacctgggttagggtaaaatgtttggagtaagagccgcatccttctctccttcccaactcccagggcagccttaggcaaggtgtactgccctgtgcctctgttatcacaaggtcacctctacacggtccggttcttgaatccagaggagggacagcctgcctgccttcaggtcccctacacatctcagcactgaggccatgcccgtctgccacacactccgctgtggtttgcctgtagactcagggttcctgtactgccaggcctttctatttcaacttttaaaacagaagaggcgttgctaggatcccagactctctgacatctaaggccgacacagggatcacctgactcctaaccggctctgcctgcccaggacctctcaagggaagcatagcgaagtgtactagcttagcaaggaccattcaattcatctttatttgtccaacaccacctggagttttaagtatgaatattaaccttgatgctttttaactattgtattaacttgctcagatttagaaatactgttttaaaagacaaagaagaaacttttttaatttctgtattttttttctgtattgtatcctatgggacactaggggttttatatggtaagacacccaaggttttggtaaaacattatcaagtatatatccagacaattcttccctagaagaaaaaacaatctctatgcctgatttaaaaaaaaagtcgaaaagaggtggattttccctttatggtgctgaaaggaagacggagactgaggagaaaataaacccgtgaggaggctcaa

SEQ ID NO: 86 - protein, 655 aa, Mus musculus
MVLYTTPFPNSCLSALHAVSWALIFPCYWLVDRLLASFIPTTYEKRQRADDPCCLQLFCTVLFTPVYLALLVAALPFAFLGFIFWSPLQSARRPYSYSRLEDKNPAGGAALLSEWKGTGAGKSFCFATANVCLLPDSLARLNNVFNTQARAKEIGQRIRNGAARPQIKIYIDSPTNTSISAASFSSLVSPQGGDGSRAVPGSIKRTASVEYKGDGGRHPSDEAANGPASGEQADGSLEDSCIVRIGGEEGGRPQEADDPAAGSQARNGAGGTPKGQTPNHNQRDGDSGSLGSPSASRESLVKARAGQDSGGSGEPGANSKLLYKTSVVKKAAARRRRHPDEAFDHEVSAFFPANLDFLCLQEVFDKRAAAKLKEQLHGYFEYILYDVGVYGCHGCCNFKCLNSGLFFASRYPVMDVAYHCYPNGCSFDALASKGALFLKVQVGSTPQDQRIVGYIACTHLHAPPEDSAVRCEQLDLLQDWLADFRKSTSSTSTANPEELVVFDVICGDLNFDNCSSDDKLEQQHSLFTRYKDPCRLGPGEEKPWAIGTLLDTNGLYDEDVCTPDNLQKVLESEEGRREYLAFPTSKSPGAGQKGRKDLLKGNGRRIDYMLHAEEGLCPDWKAEVEEFSFITQLSGLTDHLPVAMRLMVSAGEEEA

SEQ ID NO: 87 - DNA, 6246 bp, Homo sapiens
atccactgtgaaatggagtttcaaaatcacaagcttctttcccacatgaacataagactaggagcacatatggaagagtaaagttgaagggaatttggatgatgatttggcaagatgctgtaagtggattaggcaaggctgctaaagagagacaggaaatggctgtgttagtgcttcggacctccgttagaagtagcagttcatggtgttgaaggtttgttgttgaaaataccttcctgaggcacctcttcattgtgcttattcttcccacaacattgtgccaagacactgtcaagcctgggcctgtcaaagcctgggatgtcaaaggcctgggtctttctcatcctgctgtaatgtattgatagctgggggctctgcagaaggacaagggaaaggtgggatagtaacatctttttgagggaagaattggcttcctttcttgaaagtggtgaaggtacagcatatagctgcatggaagaaacagtaatcggatggctaccttctacattttgtattaggaaacaaagtccattgtaagagtccatgttgatcttggaaatagaaggattgaaaaaagctaaatttccacaaagaacaagaacttgaccatctcctttttgatctgaagactaggggacaatggatatcatagagacagcaaaacttgaagaacatttggaaaatcaacccagtgatcctacgaacacttatgcaagacccgctgaacctgttgaagaagaaaacaaaaatggcaatggtaaacccaagagcttatccagtgggctgcgaaaaggcaccaaaaagtacccggactatatccaaattgctatgcccactgaatcaaggaacaaatttccactagagtggtggaaaacgggcattgccttcatatatgcagttttcaacctcgtcttgacaaccgtcatgatcacagttgtacatgagagggtccctcccaaggagcttagccctccactcccagacaagttttttgattacattgatagggtgaaatgggcattttctgtatcagaaataaatgggattatattagttggattatggatcacccagtggctgtttctgagatacaagtcaatagtgggacgcagattctgttttattattggaactttatacctgtatcgctgcattacaatgtatgttactactctacctgtgcctggaatgcatttccagtgtgctccaaagctcaatggagactctcaggcaaaagttcaacggattctacgattgatttctggtggtggattgtccataactggatcacatatcttatgtggagacttcctcttcagcggtcacacggttacgctgacactgacttatttgttcatcaaagaatattcgcctcgtcacttctggtggtatcatttaatctgctggctgctgagtgctgccgggatcatctgcattcttgtagcacacgaacactacactatcgatgtgatcattgcttattatatcacaacacgactgttttggtggtaccattcaatggccaatgaaaagaacttgaaggtctcttcacagactaatttcttatctcgagcatggtggttccccatcttttatttttttgagaaaaatgtacaaggctcaattccttgctgcttctcctggccgctgtcttggcctcctggctgcttcaaatcatcatgcaaaaagtattcacgggttcagaagattggtgaagacaatgagaaatcgacctgaggagcaaaacaaaggcatcagctcttacaccaaaagagttaacgctgtaaccaaaggtatagttttgttttttattttaggagaactgactggtaaatgaagaaatggaccaaattttgtgtaaacgattagaaagatgaacaaagtattgccctttgactggttttcttcttcatcctgagaaagatacattctcttgcagctcttcattcattggtgacaagcccccaccccgggactttactaatgagcttgttaaagaggtgccaaagaacatattcctcctttctttattctttctccaccaaaaccctctacttcagaattttttcaggatatttttcagcccaaggtcagaagaatgtgttaatattttaaataaaatatctggacatctacagccactgtgtaaatagaactgcctaatgttgagagtggtttttagcattaggtttagcaagggggagatccgtgggttgtgcgtcagctttgggtgaattttgtttctaccctgtcacggggaaagttcgggttgagtccaggagtgcacactgctgctgccacccaatgcgctacatatcacttttttttgttttgttttgttttgtttttaaaagatcattttatcttaaaaaggaaagctgatccaagtaaacacgaaagtatttgacacaccccacagattttacatgtgtgtaaatgtttcactttaaaatctctatgacagatacacaggaaacatgagatggtttctgctaatgagtggcccttgagtacacacttagatgctgtctgccctgtaaatttggatctggtgccccagggcagtcaactcttctagcacaggctgaaaacacgtgtgtgtcaactgaggttcacaccacttggggaatgagcctgttttctttccaggtcaggccctggtgtgagatcaattttagggctcctaattggagcacagaaatgtatttggtcaaatttcattgaaggtgatttcttcctttttctgttgtactttatggaaaaaccaagatggaacctgaaagttaatgtatccttgcttttagcaagagactcagtttcttataactagtcctaaggacatatgccgcaattgagtaattttacttttaccctatgaaaataacatggtgctgcactataatatactctcggaatcagtgtgttagttacagctatacagtgaagggggataactgtttctaaatttctttaaggtgatgccaaaaaatggattaaaaaatctgatcagatttaatgttaccagatttagtgcattatttaatgctattggatcttttggataattcttggcttaatttcttagctacttgaaggttaatttgcaagacttttaaaaccttagaaaagttttaaggttgcaaagttatcaacactggggcagagggtggagaggccaatgcgggtagaaggaggcagttatgtttatattgaaggtgaaatttttctttcatttagaatggaaaacatccccaaatgtatcattataaactagtcagccttgactacacaaaattgacctttaagttgcttgagaaaaacacaatgcaaatcgttcagaagggtcaacatcctttggtgctaaaatcttgtgtatgttttcagaatggctttttctgtatgttatagaataatcactaaaggaaaggtagttgaatttaaagtcatgaagcaagactctttaattcagttattttaaacaagaattaaaaccccaaacatccttggcaggctttgaagcacacagaattttctagtatttcttattaaatacaccaataataaacatatacttagtttacctgatgagtttagacataattctatataatgttcgcttatattcatttattaactagcaacacttgtgcaagaacaaggtattctgcttgaaaacactgatttttaagttcctgttgcccgattgtaaagaaaatcatctgacacagaagcctggattttgctctcctaacactggtgttttccttgagcctctaattcaaataaaacaaactgatacaccttacagccttattcatctgtgtctggataactcacttttcatctattgaataagaaaaagtgtttaaacttaataaaataaaaaattgtgcaccaattactacagatccttgaaacagtaagccttggattttagtcaagtaatccagtttttttttaatttaaaaaaaaccatcaccttttaaagaactttaaaaacacctgtgatgccaatgtggacattgccgctgccatgtctttacacgcatgtgtcgtatagctctgtcatcgagttgaggaagtccatggtttgcaaataagggtgggggaatcaaaatttcaagatggaaaaataaaacaaatagtaaaacccatttacatgtaggaaagaaaagttgagcttcaacccaatgtgccactttgatataaaacaagtatgatatatatataaactgaagatataaaacaagtatgatcttgcctttgaaatcataaatgtttggggtacctgttctttgccagttaagatacatatcttattatctttgtttttttcaagtctatgctcctgtttgaagcttttcctgtaatttaggttgtctgtgaaatacctataacatataattcctatagagtatgccacattttttttctaactcatttcaaatgaaattctctcagattctagtttttgagcttgtccactagatctgaaaataaagcatcctttcctgagtccacttgaactaattgtgaatttgttacttaatttactggcatcttgggaaacaagttttgctgtggcaggaaggctgttttgagagtgagcgttgagtctactctggtttgtggatgacattgcattagggttatttcctgtattaccagtgcccccttgtggcaatatactttatgacttggaatgcaaacaccacttttaaaagcctgttttcaagtttttgaaaggcatttgttttctgttgtttgccaataatctgaaagtatcatgtgaaaggaattattttaaaagcattttaaacctttcccaaaggtaaaatgtgctcagattataggcacttgtaattctaaccttgtatccatttgatcaaagaaaatttgtagaacatttttacctctcatgatcacaatcattagtgtctccctttataacgacctatgttttgtttactttgaaacaccaaatttgttgtcttggttttttttacatgacagaactcatgcagtttcttttttagttgaaagttcacatcttgccccttgaatagtttgaacatttctttcttaattcttttttgtttttgttttgcactgtaaaactgatgaaaactttaagccaatcttaatgcactagcctctgtagtgtaaggaagtgtgagaaggagtttcttaatgagtttacacgtttaaatcaagcatattataagttatatgccatgtgttgaaggcttttctatttatatatataaagatttttagtatttgtttttagtgtccttgtgttgcaataatttaactcctttgactcagttgctgaaaatatttcttctattaagaagtgctcttgacttcaacacccaaacactgaaaatcatgtcaatgttacccaaagataagtttttatacagatacacaccttataagttctgatttattttcttactcattatgctgtgttctaaataaatcatgaatgagaagagtgctttattgtgaaattatttaaaactgtcctttaaaagaaaaagaggaaacgatgaacaaaaactaatctaattgccaagttagaattcattatttaatttacctcctatgcaatgattaatgctgcaaaatgtatggttatgttaccgtatattcacaaaagaaatattattacaaggtttcagaggtagccaattgcattcttttggaaatttactgtactgtttcaatgtgttaagtgccttgttgtaaagtaaaattttaagtctagattcattattttcctgacatatatttttcattatgatatctactgtatgctattgtgatagttttatgaaatgcttacattttaatcaaatatgtaaatttcagaagctcttttttcctacccaccagtaccttaatcattggtttatcacattggattcaaattcaggttccttttttgataaagaggaaatttgttttccatgcttgtgattttgtgtttgtaaacatttcaggaggaatttaggagtgtaactatagtttatacttctataatgttgtcttacatttacatttttaagtgcctaattgttaaaactgatttatgtttctcttctaaagaggatatgtgtacttggtttctaatctctttggttttgctttttaaaaaatgcaagagcctatactttgtatttacctaataaaccacagtgacatcaacagtcttttcaaaattaaaaaaaaaaaaaaaaa

SEQ ID NO: 88 - DNA, 5791 bp, Mus musculus
tgtctgtcctcggttgaagcttcttggatgcatgaccaccaggctaggagcacatgccggaatgttagatatgttcagtcaactaggttggagtgggtagacgccagccatcgctgcgtgagggcctgggtatcttcctcaggagggatccatggtagagagagttgctcttggattcatcttgctgagggtcctcctcaggagttcattcttcccacggccttttctaaaggcaccgaagagagcattggtcaggccagaggaacgctggcttcctcactccctctactagggtcttggtacttggagagactttcacagggacaagaggaagaaggaacaataacatcatctgtgggaaagagaaccagcttccttgtggtgactacacagcaggttgttgtatggaaggactaggaaccagcaggccaccgactacatttcgtatcagcacacatcatcagttctaagagtccatgttgatctcgcaagtagaaggatttaaaagagaaaggttcccacagaaaccaagaaccttaccatctcctttagccctggagggtggcggacaatggatatcatagagacagcaaaacttgaaggtcacttggaaagtcaaaccaacgactccaccaacacttacacaagccccaccgaagctgtagaggaagagggcaaaaatggcaagggcaaacccaagaccttatccaacgggttacgaaagggtgccaaaaaatacccggactacatccagatttccatgcccaacgactccaagaacaagtttcccctggagtggtggaaaacaggcatcgcctttgtgtatgcgctcttcaacctcatcctgacaaccgtcatgatcaccgtcgtgcacgagagggtccctcccaaggagctcagccctccgctcccagacaagttttttgattacttcgaccgggtcaaatgggcattttctgtatcagaaataaacgggatggtattggttgggttatggatcacccagtggctctttctgcgttacaagtcaatagtggggcgcagattcttcttcatcatgggaactttatacctgtatcgctgtataactatgtatgtcactacgttacctgtgcccggaatgcacttccagtgtgctcccaagctcaatggagactctcaggcaaaaatacagcggattctccggctgatttctggcgggggactgtctatcacgggatctcacatcctgtgcggagacttcctcttcagcggccacactgtcgtgctcacacttacttacttgttcatcaaagaatattcacctcgtcacttctggtggtatcacttggtctgctggctgctgagtgcggctgggatcatctgcattctcgtagcgcatgaacactataccgtggacgtcatcattgcttactatatcacaacacggctgttttggtggtaccattccatggccaacgaaaagaacttgaaggtctcttcccagacgaacttcttgtctcgggcttggtggttccccatcttctacttttttgagaagaatgtgcaaggctcaattccttgctgcttttcctggccgctgtcctggccccctggctgcttcaagtcatcgtgcagaaagtattcccgggtccagaagatcggggaggataatgagaagtctacctgagctgccaacccggcagccgctctcacaccaaaagagtccgtgctgcaaccgaaggcacgtgcggctttatatttattttcagagaactgactggtaaaatgaagtggaccaaattttatgcaaaagattggagcgatgaagtattacctttggcttttttttattcatcccaagaaacatatattttcctgcagctcttcgttcattgatgacaaagcccccacactggagttctgaagaggtggcaaagaacacgccgagcctctcctcgccttccttcacttccacgttctttccagattgctttttttctcccttcaaggtcagaagagtttgctaacgttttgaataaaatgtctggatatatacagccactgtgtaaatacagtcagttgatggcagtgggttttcaccacagagcttagtaaggggcagcctgcaggtggggcaaggtcattagggtgatgtctggtgtcccctcccctcccccgccacccattatgcatgacttttactctttttgggggaggggggttggcttttggtttttcaagacagggtttctctgtgtagccttggctgtcctggaactcactctgaagaccacacccggcctcttttttttttttttttttaaacctggtaattttagtttttaaagaaagagtattgcaaggtcatgtgaaagagacacacccatggatattttttccctgtgtggggatttttccactttcaagtgtccgtggcagataaccggcaggctccgggcttttcctgctatacccgttagatgctgcctgccgggagcccaaggcttgttaaccgcaccagcacaggctgaaaccagtgtgaacagatgctcacactggagagcaagcttgtgctctctgctagcttgggccctgccgtgccatgccgtgatgccgttacttattggaccctagagaaacatttgggcacattccatgaaaacagcttctttttattttaatatcgtgtgtctcgggaatgcaagatggatcccaaaggttttactgtttccctgccttttagcaagacccccccccccagcgctccttagggcgtgcaccaccgctgagcaactcacttcaccctgtgcacataacacgcggtgctgcccttgaacatgtccctggcatcagctcttaacctcagccatgtaggaatgagggacaactgttatctcatgtgaaaagatgggtctctaagatctggtgggtttaagaacatcggactcagttagaaggttactcagtgctgggatatcatagtccttgcctttggttgagatctactgaaagattagtttgtggaacttgtagaaaaacttctaaggattcacagcatgggagcagccagtggaaggggtggttacattctcatgggtatggcatagagaacatccctgggttcgtccctgtaagctagtcggccttgattgtttaaaattaaactttaaattgcttgggagaaaaaaaaaagcacaatacagatgtgttcagaagggccattgtcttctggtgctataattctgtgtgtgtgtttcagagggtctcgttttctgtgttactgggtgggtaaaggataggatgctaaacccaaagacacgagcataaaaatctaagtctgactatcctcacaagagcagagaccctaagtgtctttggcagactttgaaatgcagggacttttttttttcaagtatttttatcgagtaagccaataataaattcatacccagtttaccttgtactagacaattcggtacaactcctcacgtgttcatttcctcagctctgtggaaagctctgctccgaacactagtttaccttcagggtctgattatgagagaatcatttggcgctgaagcctggcctttgctctgcgagtctctcactgaaacaaaacactgaaacaaaacacctgaaaagccttatccagcctggctaggattctccatgattcttcaaccgaggaaaatgcttagacattgttacatttcatgttgattgttgggaacccttcaaacagaaaactttgcatttagtcaggtaaaccaggctgttggaaacaataccatcactttcttaaggtttgaaaatacccatgagccctgtgtggaccctgctgttccctgttcttgtagtagctgtgattcctaataggtaccgatggggatgcaagatgacaactcattgcagtgcaggaaagtaagacttcctttaagatggtctcatctcactgtgtagcccagattggcctccaaccttcagccctcctgtatcagccccctgggtgctgggattgcaggtgtgtttccatgcctacccaggcacagcaggacatggtaccagcaccttttctttgcccattaagatgcagattttacccattaagtgtgggtaaggcgcaagcgggcgcctgtgctcctgttggaaggttttccccataatatagagcttctggaaaacgtccacaaatatagaacacacatgtttttcctgaaccttcccaatgggactggagttctctgagactccgggtttgagctcctccactagatcgggaaaaggaggtatccttcccccagtgacccctgagctaattttgactgtcttttgggttgctgtcatcacaggaagcagctttttgctggggacacaaggttggctgagagtcagctcttgcaatccctgggcaggtttgtggccatccatcccttgctgtgttgccagcgcccccttgtggtgatatgtcatgtctgaaatccaaatgccactttaaaaatatattttcaaggtttctctaaagtttatctgttgggccttttgttgttgctgttttcagggttttttgccaaaagttgaaattcccatgggaaaggagctattttaaagacattcttatccttcccaaagggaaagttctcagaatttgagtcaaagaaagttgaaggtcacatttacctcttatgtcacaatcattcgtgtctggcccatagctacagactttgtttacttcaaaacccaaagtcagctgtctcatttttatttatgtgccagaactcatcatttcacttgtaattgaaaggtcttaccttgttccataaaatattttaaactctttgttaaatgttttgattttatactataaaactgatttaaaaattaagccagtcttaacacactcaccctgtctgtagtgtaaggaagtgtgagaaggaagttcttttttattgtttgtttgtttgtttgtttgtttgtttgagacagggtttctttgtgtagccctggctgtcctggaactcacgcagtagaccaggctggccttgaactcagaaatccgcctgcctctgcctctgcctctgcctcctgaatgctaggattaaaggcatatgccaccaccgcctgacgagaaggaatttcttaataaatgagtttacatgtctaaatcaagtgtaagttatatgcgttgtgttggaaccttttctacgtttatgtataaagatttcttatattattatttttaatgccattgcgttgcaataatttagctcctgactcatttgacaaaaatacatcttttatgaagaagtactcttgactttgatatctgtacactgaaaatcatgtcagaagtacccaaagataagtttttatacagatagacgcttcgtaagcgctggcttgttacccactaactaactgtactgtgttctcaatgaatcatgaaagacaagagtgggtcactgtagaattattttaaactgtcctttaaaagagaagaaactataagctaatctagttgccaagtaggaattccttatttaatttacctcctatgcaatgattaatgctgcgaaatgtatggttaataagttacagtatattcacagaagaaatatctttccaaggtgtaagaagcagccagttgcattcttttggaaatttactatactgtttcaatgtgttaagtgccttgttgtaaagtaaaattttaagtctcgagttcattattttcctgacctatatttttcattatgattatctactgtgtgctgttgtaatcattttattaaatgcttacattttaatcaaa

SEQ ID NO: 89 - protein, 55 aa, Homo sapiens
DAEFRHDSGYEVHHQKLVFFAEDVGSNKGAIIGLMVGGVVIATVIVITLVMLKKK

SEQ ID NO: 90 - DNA, 19 bp, Artificial
  human SMS2 Fw primer
tcaatggagactctcaggc

SEQ ID NO: 91 - DNA, 19 bp, Artificial
  human SMS2 Rv primer
ccgctgaagaggaagtctc

SEQ ID NO: 92 - DNA, 18 bp, Artificial
  human G3PDH Fw primer
ccttccgtgtccccactg

SEQ ID NO: 93 - DNA, 19 bp, Artificial
  human G3PDH Rv primer
accctgttgctgtagccaa

SEQ ID NO: 94 - RNA, 19 nt, Artificial
  SMS2-i4 sense strand of duplex portion
gcagauuguuguugaucau

SEQ ID NO: 95 - RNA, 19 nt, Artificial
  SMS2-i4 antisense strand of duplex portion
augaucaacaacaaucugc

SEQ ID NO: 96 - RNA, 19 nt, Artificial
  CTR-i sense strand
uucuccgaacgugucacgu

SEQ ID NO: 97 - RNA, 19 nt, Artificial
  CTR-i antisense strand
acgugacacguucggagaa

SEQ ID NO: 98 - RNA, 27 nt, Artificial
  SMS2 sense strand
auacauaguuauacagcgauacaggnn

SEQ ID NO: 99 - RNA, 27 nt, Artificial
  SMS2 antisense strand
ccuguaucgcuguauaacuauguaunn

(19) United States
(12) Patent Application Publication
(10) Pub. No.: US 2014/0256793 A1
(43) Pub. Date: Sep. 11, 2014

(54) MEDICAMENT FOR DISEASES ASSOCIATED WITH AMYLOID beta AND SCREENING THEREOF

(71) Applicants: National University Corporation Hokkaido University, Sapporo-shi, Hokkaido (JP); Shionogi & Co., Ltd., Osaka-shi, Osaka (JP)

(72) Inventors: Kohei Yuyama, Sapporo-shi (JP); Yasuyuki Igarashi, Sapporo-shi (JP); Susumu Mitsutake, Sapporo-shi (JP); Tetsuya Yoshida, Osaka-shi (JP)

(21) Appl. No.: 14/351,530

(22) PCT Filed: Oct. 12, 2012
(86) PCT No.: PCT/JP2012/006545

§ 371 Date: Apr. 11, 2014

(30) Foreign Application Priority Data
Oct. 14, 2011 (JP) 2011-226786

(51) Int. Cl. C12N 15/113; C12N 9/16; C12Q 1/44; A61K 31/713; A61K 31/7088

(52) U.S. Cl. CPC C12N 15/1137; A61K 31/713; A61K 31/7088; C12Q 1/44; C12N 9/16
USPC 514/44 A; 536/24.5; 435/19; 435/196

(57) ABSTRACT

The present invention is directed to providing a medicament for treating or preventing a condition, disorder or disease associated with amyloid β. Provided is a method for screening a medicament for treating or preventing a disease associated with amyloid β, the method comprising: A) subjecting at least one of elements selected from the group consisting of 1) exosome; 2) neutral sphingomyelinase 2 (N-SMase2); and 3) sphingomyelin synthetic enzyme 2 (SMS2), and a candidate of the medicament in a condition in which they can interact with each other; and B) examining an influence by the candidate of the medicament to the element, wherein at least one of the elements is used as an index for determining as to whether or not the candidate is the medicament.

### TECHNICAL FIELD

The present invention is directed to the novel usage of sphingomyelin synthetic enzyme 2 (SMS2) and neutral sphingomyelinase 2 (nSMase2, also referred to as N-SMase2 in the present invention). More specifically, the present invention is directed to a pharmaceutical composition for preventing or treating diseases (e.g., Alzheimer's disease) associated with amyloid β protein (Aβ) using SMS2 and nSMase2; a substance thereof, or a method for prevention or treatment thereof. The present invention is also directed to screening of a medicament for diseases associated with amyloid β, and a screening technique for a novel regulatory factor associated with exosome and amyloid β.

### BACKGROUND ART

Alzheimer's disease (AD) is a delayed, neurological disorder which is caused as a result of the defect and death of the nerve cell (neuron) and which is accompanied by progressive memory loss and cognitive ability. AD is characterized, from the pathological point of view, by widespread extracellular deposition of amyloid fibril that is constituted of amyloid β protein (Aβ) in the nerve cells. The Aβ is generated as a physiological metabolic product through contiguous processing of an amyloid precursor protein (APP), followed by secretion to an extracellular environment in the brain. The level of the extracellular Aβ in a steady state is controlled by the balance between the generation thereof in the brain and degradation/clearance. Some pieces of evidence suggest the association between the accumulation of Aβ, which contributes to the metabolic imbalance thereof, and the onset of the pathological condition of AD (Non Patent Literature 1). Indeed, the increase of the Aβ level results in the increase of the formation of soluble oligomer, which is known to induce neural toxicity directly, and insoluble fibril (Non Patent Literature 2; Non Patent Literature 3). In cases of familial AD, the hereditary change of causal gene such as APP and presenilin seems to promote the aggregation of Aβ due to significant increase in the Aβ generation (Non Patent Literature 4). On the other hand, with regard to sporadic AD, which is a primary form of this disease, the decrease in the Aβ removal level in the brain has been recently reported (Non Patent Literature 5). This corresponds to the existing finding that Aβ is decreased in the cerebrospinal fluid (CSF) of AD patients and patients suffering from a medium degree of cognitive disorder (Non Patent Literature 6) and it suggests, for example, decrease in catabolism due to the decrease in proteolytic degradation, or a confused state of Aβ clearance through reduced flow over the blood-brain barrier into the CSF. However, there is still room for discussion with regard to the strict clearance process that is damaged in delayed AD.

Recently, it has been reported that a part of the extracellular Aβ is bound to a follicle referred to as exosome (Non Patent Literature 7). The exosome is a specific subtype of secretory, small membrane vesicles (of 40 to 100 nm in diameter) derived from various cell types (Non Patent Literature 8). They correspond to intratubular follicle (IL) of an endosome multivesicular body (MVB), which fuses together with a plasma membrane via exocytosis (Non Patent Literature 9). The well known functions of the exosome are to remove proteins and lipids which are obsolete ormisfolded, and to secrete them through the excertory system such as urine or intestines (Non Patent Literature 10; Non Patent Literature 11). In addition, accumulated pieces of evidence show that these function as a shuttle for the intracellular delivery of a vehicle comprising a specific combination of proteins, lipids and RNAs. With regard to the expression of the disease state of AD, the researches so far carried out by the inventors (Non Patent Literature 12) demonstrate that an exosome derived from pheochromocytoma PC12 strongly induces an insoluble Aβ fibril under a circumstance of endocytosis incompetence, which indicates a pathological change of an early stage that is seen in a nerve cell of an AD brain. In addition, it has been reported that Alix, which is a marker protein of exosome, is accumulated in the Aβ plaque that is found in the brain of AD patients (Non Patent Literature 13).

Sphingomyelin synthetic enzyme (SMS) is an enzyme for synthesizing sphingomyelin (SM), which is a sphingolipid that is present the most in a cell membrane, and the sphingomyelin synthetic enzyme plays an important role in cell death and survival (see Non Patent Literature 14 and 15). The SMS was identified in the year of 2004, and the two types, SMS1 and SMS2, are known to exist. It is known that the SMS1 expresses in a Golgi body, and is associated with the de novo synthesis of SM. On the other hand, the SMS2 is expressed in a Golgi body and a cell membrane, but the details are not known regarding its physiology (see Non Patent Literature 16). Non Patent Literature 16 to 19 only suggest the possibility of association with artherosclerosis. In addition, part of the inventors found that the SMS2 is associated with metabolic syndrome, and also disclosed a method for the treatment or prevention therefor (Non Patent Literature 20).

### CITATION LIST

### Non Patent Literature

Non Patent Literature 1: Hardy J et al., Science (2002) 297: 353-356
Non Patent Literature 2: Hardy J et al., Trends Pharmacol Sci (1991) 12: 383-388
Non Patent Literature 3: McLean C A et al., Ann Neurol (1999) 46: 860-866
Non Patent Literature 4: Selkoe D J, Science (1997) 275: 630-631
Non Patent Literature 5: Mawuenyega K G et al., Science (2010) 330: 1774
Non Patent Literature 6: Blennow K et al., Nat Rev Neurol (2010) 6: 131-144
Non Patent Literature 7: Rajendran L et al., Proc Natl Acad Sci USA (2006) 103: 11172-11177
Non Patent Literature 8: Simons M et al., Curr Opin Cell Biol (2009) 21: 575-581
Non Patent Literature 9: Fevrier B et al., Curr Opin Cell Biol (2004) 16: 415-421
Non Patent Literature 10: Keller S et al., Kidney Int (2007) 72: 1095-1102
Non Patent Literature 11: Gonzales P A et al., J Am Soc Nephrol (2009) 20: 363-379
Non Patent Literature 12: Yuyama K et al., J Neurochem (2008) 105: 217-224
Non Patent Literature 13: Rajendran L et al., Proc Natl Acad Sci USA (2006) 103: 11172-11177
Non Patent Literature 14: Yamaoka et al., the Journal of Biological Chemistry, 279, 18688-18693, (2004)
Non Patent Literature 15: Huitema et al., the EMBO Journal, 23, 33-44, (2004)
Non Patent Literature 16: Tafesse et al., The Journal of Biological Chemistry, 281, 29421-29425, (2006)
Non Patent Literature 17: Park et al., Circulation, 110, 3465-3471, (2004)
Non Patent Literature 18: Liu et al., Arteriosclerosis, Thrombosis, and Vascular Biology, 29, 850-856, (2009).
Non Patent Literature 19: Liu et al., Circulation Research, 105, 295-303, (2009)
Non Patent Literature 20: Mistutake S. et al., J Biol. Chem. 2011 Aug. 12; 286(32): 28544-55. Epub 2011 Jun. 13

### SUMMARY OF INVENTION

### Solution to Problem

The present invention is directed to a screening method of a regulatory factor such as a medicament, focused on the association of the metabolism of sphingomyelin with exosome and amyloid β (Aβ), and to a medicament and the like obtained thereby. In addition, the present invention provides a method for associating with a condition, disorder or disease associated with amyloid β of synthetic enzyme of sphingomyelin, and for prevention or treatment thereof.

The present invention is directed to a treatment medicament or preventive medicament for a disease (e.g., Alzheimer's disease) associated with amyloid β (Aβ) due to suppression of the synthesis of sphingolipid via SMS2 or nSMase2.

Accordingly, more specifically, the present invention provides the following.

In one aspect, the present invention provides a method for screening a treatment substance or prevention substance for a disease associated with amyloid β, comprising: (1) allowing protein of neutral sphingomyelinase 2 (N-SMase2) and/or sphingomyelin synthetic enzyme 2 (SMS2) to contact with a test substance; (2) comparing enzyme activity of the protein of the N-SMase2 and/or SMS2 to which the test substance has been contacted, with enzyme activity of protein of the N-SMase2 and/or SMS2 to which the test substance has not been contacted; and (3) when the enzyme activity of the protein of the N-SMase2 to which the test substance has been contacted is increased compared to the enzyme activity of the protein of the N-SMase2 to which the test substance has not been contacted, and/or the enzyme activity of the protein of the SMS2 to which the test substance has been contacted is decreased compared to the enzyme activity of the protein of the SMS2 to which the test substance has not been contacted, selecting the test substance as a treatment substance or prevention substance of the disease associated with amyloid β.

In another aspect, the present invention provides a method for screening a treatment substance or prevention substance for a disease associated with amyloid β, comprising: (1) allowing a cell to contact with a test substance; (2) comparing expression of N-SMase2 and/or SMS2 in the cell to which the test substance has been contacted, with expression of N-SMase2 and/or SMS2 in a control cell to which the test substance has not been contacted; and (3) when the expression of the N-SMase2 in the cell to which the test substance has been contacted is increased compared to the expression of the N-SMase2 in the control cell to which the test substance has not been contacted, and/or when the expression of SMS2 in the cell to which the test substance has been contacted is decreased compared to the expression of SMS2 in the cell to which the test substance has not been contacted, selecting the test substance as a treatment substance or prevention substance of the disease associated with amyloid β.

In another aspect, the present invention provides a method for screening a treatment substance or prevention substance for a disease associated with amyloid β, comprising: (1) allowing a cell to contact with a test substance; (2) comparing an exosome secretion level in the cell to which the test substance has been contacted, with an exosome secretion level in a control cell to which the test substance has not been contacted; and (3) when an exosome secretion level in the cell to which the test substance has been contacted is increased compared to an exosome secretion level in the control cell to which the test substance has not been contacted, selecting the test substance as a treatment substance or prevention substance of the disease associated with amyloid β.

In one embodiment, the cell and the control cell used in the method according to the present invention are nerve cells.

In another aspect, the present invention provides a pharmaceutical composition for treating or preventing a disease associated with amyloid β, comprising a substance for increasing enzyme activity or expression of protein of N-SMase2. In this aspect, the present invention may be provided as a substance for increasing enzyme activity or expression of protein of N-SMase2, for treating or preventing a disease associated with amyloid β. Alternatively, in this aspect, the present invention may be provided as a method for treating or preventing a disease associated with amyloid β in a subject, the method comprising: administering an effective amount of the substance for increasing enzyme activity or expression of protein of N-SMase2 to a subject in need of such a treatment or prevention.

In yet another aspect, the present invention provides a pharmaceutical composition for treating or preventing a disease associated with amyloid β, comprising N-SMase2. In this aspect, the present invention may be provided as N-SMase2 for treating or preventing a disease associated with amyloid β. Alternatively, in this aspect, the present invention may be provided as a method for treating or preventing a disease associated with amyloid β in a subject, the method comprising: administering an effective amount of the N-SMase2 to a subject in need of such a treatment or prevention.

In yet another aspect, the present invention provides a pharmaceutical composition for treating or preventing a disease associated with amyloid β, comprising a substance for suppressing enzyme activity or expression of protein of SMS2. In this aspect, the present invention may be provided as a substance for suppressing enzyme activity or expression of the protein of SMS2, for treating or preventing a disease associated with amyloid β. Alternatively, in this aspect, the present invention may be provided as a method for treating or preventing a disease associated with amyloid β in a subject, the method comprising: administering an effective amount of a substance for suppressing enzyme activity or expression of the protein of SMS2 to a subject in need of such a treatment or prevention.

In one embodiment, the pharmaceutical composition used in the present invention is a nucleic acid.

In another embodiment, the above-mentioned nucleic acid used in the present invention is an siRNA and/or antisense nucleic acid.

In a detailed embodiment, the above-mentioned siRNA used in the present invention is selected from the group consisting of siRNAs in the following (a) to (p):

(a) an siRNA wherein one strand of the duplex RNA portion is a base sequence set forth by SEQ ID NO: 1 and the other strand of the duplex RNA portion is a base sequence set forth by SEQ ID NO: 2;
(b) an siRNA wherein one strand of the duplex RNA portion is a base sequence set forth by SEQ ID NO: 3 and the other strand of the duplex RNA portion is a base sequence set forth by SEQ ID NO: 4;
(c) an siRNA wherein one strand of the duplex RNA portion is a base sequence set forth by SEQ ID NO: 5 and the other strand of the duplex RNA portion is a base sequence set forth by SEQ ID NO: 6;
(d) an siRNA wherein one strand of the duplex RNA portion is a base sequence set forth by SEQ ID NO: 7 and the other strand of the duplex RNA portion is a base sequence set forth by SEQ ID NO: 8;
(e) an siRNA wherein one strand of the duplex RNA portion is a base sequence set forth by SEQ ID NO: 9 and the other strand of the duplex RNA portion is a base sequence set forth by SEQ ID NO: 10;
(f) an siRNA wherein one strand of the duplex RNA portion is a base sequence set forth by SEQ ID NO: 11 and the other strand of the duplex RNA portion is a base sequence set forth by SEQ ID NO: 12;
(g) an siRNA wherein one strand of the duplex RNA portion is a base sequence set forth by SEQ ID NO: 13 and the other strand of the duplex RNA portion is a base sequence set forth by SEQ ID NO: 14;
(h) an siRNA wherein one strand of the duplex RNA portion is a base sequence set forth by SEQ ID NO: 15 and the other strand of the duplex RNA portion is a base sequence set forth by SEQ ID NO: 16;
(i) an siRNA wherein one strand of the duplex RNA portion is a base sequence set forth by SEQ ID NO: 17 and the other strand of the duplex RNA portion is a base sequence set forth by SEQ ID NO: 18;
(j) an siRNA wherein one strand of the duplex RNA portion is a base sequence set forth by SEQ ID NO: 19 and the other strand of the duplex RNA portion is a base sequence set forth by SEQ ID NO: 20;
(k) an siRNA wherein one strand of the duplex RNA portion is a base sequence set forth by SEQ ID NO: 21 and the other strand of the duplex RNA portion is a base sequence set forth by SEQ ID NO: 22;
(l) an siRNA wherein one strand of the duplex RNA portion is a base sequence set forth by SEQ ID NO: 23 and the other strand of the duplex RNA portion is a base sequence set forth by SEQ ID NO: 24, which is a complementary sequence thereof;
(m) an siRNA wherein one strand of the duplex RNA portion is a base sequence set forth by SEQ ID NO: 25 and the other strand of the duplex RNA portion is a base sequence set forth by SEQ ID NO: 26, which is a complementary sequence thereof;
(n) an siRNA wherein one strand of the duplex RNA portion is a base sequence set forth by SEQ ID NO: 27 and the other strand of the duplex RNA portion is a base sequence set forth by SEQ ID NO: 28, which is a complementary sequence thereof;
(o) an siRNA wherein one strand of the duplex RNA portion is a base sequence set forth by SEQ ID NO: 43 and the other strand of the duplex RNA portion is a base sequence set forth by SEQ ID NO: 44;
(p) an siRNA according to any of (a) to (o), wherein one to several nucleotides are added, inserted, deleted or substituted in one or both of the base sequences, and having an activity of suppressing the expression of SMS2.

In a further aspect of the present invention, the present invention provides a screening method for a medicament for the treatment or prevention of a condition, disorder or disease associated with amyloid β. This method comprises the steps of: A) subjecting at least one of elements selected from the group consisting of 1) exosome; 2) neutral sphingomyelinase 2 (N-SMase2); and 3) sphingomyelin synthetic enzyme 2 (SMS2), and a candidate of the medicament in a condition in which they can interact with each other; and B) examining the influence by the candidate of the medicament to the element, wherein at least one of the elements is an index for determining as to whether or not the candidate is the medicament.

In one embodiment, said condition, disorder or disease is due to the polymerization or fibrillation of amyloid β.

In one embodiment, said condition, disorder or disease is one or more selected from the group consisting of Alzheimer's disease, retinal disease (e.g., age-related macular degeneration (also referred to as age-related macular retinopathy), glaucoma and the like) (see Journal of Pharmacological Sciences, Vol. 134 (2009), No. 6, 309-314 and the like).

In one embodiment, said step A) is a step of subjecting a cell and said candidate to a condition in which they can interact with each other, and said step B) is a step of examining the secretion level of said exosome. Herein, the secretion level from the cell of the exosome is used as an index for determining as to whether or not the candidate is a medicament.

In one embodiment, said cell is a nerve cell.

In a certain embodiment, said element includes exosome, and there is a step included therein of contacting the exosome with Aβ1-40 and/or Aβ1-42 in the presence or in the absence of said candidate of the medicament, wherein the amount of at least one of the exosome, the Aβ1-40, the Aβ1-42 and Aβ polymer is used as an index for determining as to whether or not the candidate is the medicament.

In another embodiment, said element includes exosome, and there is a step included therein of contacting the exosome with Aβ1-40 and/or Aβ1-42, and microglia, in the presence or in the absence of said candidate of the medicament, wherein the intake of the exosome and/or the Aβ1-40 and/or the Aβ1-42 by the microglia is used as an index for determining as to whether or not the candidate is the medicament.

In yet another embodiment, there is a step included therein of examining the activity of NSMase2 and/or SMS2 in the presence or in the absence of said candidate of the medicament, wherein the decrease in the activity of the N-SMase2 and the increase in the activity of the SMS2 are used as an index for determining as to whether or not the candidate is the medicament.

In another aspect, the present invention provides a pharmaceutical composition for treating or preventing a disease associated with amyloid β, comprising a protein and/or expression vector of N-SMase2.

In yet another aspect, the present invention provides a pharmaceutical composition for treating or preventing a disease associated with amyloid β, comprising a nucleic acid for suppressing the expression of SMS2.

In one embodiment, the nucleic acid used for the pharmaceutical composition according to the present invention is an antisense nucleic acid.

In another embodiment, the nucleic acid used for the pharmaceutical composition according to the present invention is an antisense nucleic acid including a locked nucleic acid (LNA).

In another embodiment, the antisense nucleic acid used for the pharmaceutical composition according to the present invention consists of one or more of SEQ ID NO: 29 to 40.

In another aspect, the present invention provides an siRNA for treating or preventing a disease associated with amyloid β, selected from the group consisting of (a) to (p) below:

(a) an siRNA wherein one strand of the duplex RNA portion is a base sequence set forth by SEQ ID NO: 1 and the other strand of the duplex RNA portion is a base sequence set forth by SEQ ID NO: 2;
(b) an siRNA wherein one strand of the duplex RNA portion is a base sequence set forth by SEQ ID NO: 3 and the other strand of the duplex RNA portion is a base sequence set forth by SEQ ID NO: 4;
(c) an siRNA wherein one strand of the duplex RNA portion is a base sequence set forth by SEQ ID NO: 5 and the other strand of the duplex RNA portion is a base sequence set forth by SEQ ID NO: 6;
(d) an siRNA wherein one strand of the duplex RNA portion is a base sequence set forth by SEQ ID NO: 7 and the other strand of the duplex RNA portion is a base sequence set forth by SEQ ID NO: 8;
(e) an siRNA wherein one strand of the duplex RNA portion is a base sequence set forth by SEQ ID NO: 9 and the other strand of the duplex RNA portion is a base sequence set forth by SEQ ID NO: 10;
(f) an siRNA wherein one strand of the duplex RNA portion is a base sequence set forth by SEQ ID NO: 11 and the other strand of the duplex RNA portion is a base sequence set forth by SEQ ID NO: 12;
(g) an siRNA wherein one strand of the duplex RNA portion is a base sequence set forth by SEQ ID NO: 13 and the other strand of the duplex RNA portion is a base sequence set forth by SEQ ID NO: 14;
(h) an siRNA wherein one strand of the duplex RNA portion is a base sequence set forth by SEQ ID NO: 15 and the other strand of the duplex RNA portion is a base sequence set forth by SEQ ID NO: 16;
(i) an siRNA wherein one strand of the duplex RNA portion is a base sequence set forth by SEQ ID NO: 17 and the other strand of the duplex RNA portion is a base sequence set forth by SEQ ID NO: 18;
(j) an siRNA wherein one strand of the duplex RNA portion is a base sequence set forth by SEQ ID NO: 19 and the other strand of the duplex RNA portion is a base sequence set forth by SEQ ID NO: 20;
(k) an siRNA wherein one strand of the duplex RNA portion is a base sequence set forth by SEQ ID NO: 21 and the other strand of the duplex RNA portion is a base sequence set forth by SEQ ID NO: 22;
(l) an siRNA wherein one strand of the duplex RNA portion is a base sequence set forth by SEQ ID NO: 23 and the other strand of the duplex RNA portion is a base sequence set forth by SEQ ID NO: 24, which is a complementary sequence thereof;
(m) an siRNA wherein one strand of the duplex RNA portion is a base sequence set forth by SEQ ID NO: 25 and the other strand of the duplex RNA portion is a base sequence set forth by SEQ ID NO: 26, which is a complementary sequence thereof;
(n) an siRNA wherein one strand of the duplex RNA portion is a base sequence set forth by SEQ ID NO: 27 and the other strand of the duplex RNA portion is a base sequence set forth by SEQ ID NO: 28, which is a complementary sequence thereof;
(o) an siRNA wherein one strand of the duplex RNA portion is a base sequence set forth by SEQ ID NO: 43 and the other strand of the duplex RNA portion is a base sequence set forth by SEQ ID NO: 44;
(p) an siRNA according to any of (a) to (o), wherein one to several nucleotides are added, inserted, deleted or substituted in one or both of the base sequences, and having an activity of suppressing the expression of SMS2.

In one embodiment, said disease associated with amyloid β is one or two or more selected from the group consisting of Alzheimer's disease, retinal disease and age-related macular retinopathy.

In another aspect, the present invention provides a nucleic acid containing a locked nucleic acid (LNA) for treating or preventing a disease associated with amyloid β, consisting of any one or more of SEQ ID NOs: 29 to 40.

In one embodiment, said disease associated with amyloid β is one or two or more selected from the group consisting of Alzheimer's disease, retinal disease and age-related macular retinopathy.

In another aspect, the present invention provides a method for treating or preventing a disease associated with amyloid β, the method comprising: administering the pharmaceutical composition according to the present invention in an amount effective for the treatment or prevention to a subject which requires the treatment or prevention.

In one embodiment, said disease associated with amyloid β is one or two or more selected from the group consisting of Alzheimer's disease, retinal disease and age-related macular retinopathy.

In another aspect, the present invention provides a method for treating or preventing a disease associated with amyloid β, the method comprising: administering the siRNA according to the present invention in an amount effective for the treatment or prevention to a subject which requires the treatment or prevention.

In one embodiment, said disease associated with amyloid β is one or two or more selected from the group consisting of Alzheimer's disease, retinal disease and age-related macular retinopathy.

In another aspect, the present invention provides a method for treating or preventing a disease associated with amyloid β, the method comprising: administering the LNA-containing nucleic acid according to the present invention in an amount effective for the treatment or prevention to a subject which requires the treatment or prevention.

In one embodiment, said disease associated with amyloid β is one or two or more selected from the group consisting of Alzheimer's disease, retinal disease and age-related macular retinopathy.

Exosome is a dual-membrane vesicle derived from an endosome membrane released from various types of cells. In recent years, reports have been made that amyloid β (Aβ) bind to exosomes derived from nerve cells, and that marker protein of exosome is localized at the center of senile plaque of Alzheimer's disease, which suggests a possibility of some kind of role for the dynamic state of Aβ in the brain. In the present invention, exosome which was secreted from Neuro2a cells (also referred to as N2a cells) and a primary cultured cortex nerve cell was collected from a culture supernatant and an influence was examined on Aβ polymerization and Aβ intake to microglia. As a result, the exosome derived from nerve cells was found to promote the polymerization or fibrillation Aβ₁₋₄₀ or Aβ₁₋₄₂. In addition, phosphatidylserine (PS) was exposed on the surface of the exosome, which was taken in a PS dependent manner into microglia. While Aβ fibers alone are taken into microglia, the intake was promoted when the fibers were added together with exosome into a culture solution. This promotion of intake of Aβ fibers by exosome was suppressed when PS was blocked using annexin V. Furthermore, it was confirmed that the Aβ taken together with exosome into microglia was degraded within the cell. The above-mentioned result suggests the possibility for exosome to be associated with Aβ clearance. The relationship between the promotion of Aβ degeneration by exosome and the expression of the disease state of Alzheimer's disease was suggested by the present invention.

The inventors decided to perform an experiment for examining the fate of extracellular Aβ bound to exosome. Herein, the inventors demonstrated that neuroblastoma N2a and murine primary cultured cortex nerve cells compositionally released exosomes and these exosome significantly accelerated Aβ amyloid formation from a soluble form. Of special note is that when the exosome derived from nerve cells was taken into microglia, Aβ intake/degradation by microglia was enhanced. Further, the inventors demonstrated that the secretion of exosome was regulated by neutral sphingomyelinase 2 (nSMase2 or N-SMase2) and sphingomyelin synthase 2 (SMS2), known as sphingolipid synthetic enzyme. The upregulation of exosome secretion mediated by SMS2 siRNA was sufficient for the promotion of Aβ intake by microglia, and yielded a significant reduction of extracellular Aβ level in the co-culture of nerve cells and microglia cells using a transwell system. These results propose a novel mechanism for Aβ clearance mediated by exosome.

The amyloid β peptide (Aβ), which is a causal agent of Alzheimer's disease (AD), is a physiological metabolic product, and the metabolism thereof is continuously controlled in normal brains. Recent studies demonstrated that while part of extracellular Aβ binds to an exosome, which is a small membrane vesicle of endosome origin, the fate of the Aβ integrated with the exosome was mostly unknown. Herein, the inventors identified a new role for the exosome derived from nerve cells to extracellular Aβ, that is, the exosome driving a structural change of Aβ to a non-toxic amyloid fibril, and promoting intake of the Aβ intomicroglia. The Aβ taken together with exosome was further delivered to lysosome and degraded within the microglia. the inventors also found that the blocking of phosphatidylserine on the surface of the exosome by annexin V prevented the intake by exosome as well as the intake of Aβ into microglia. In addition, the inventors demonstrated that the secretion of exosome derived from nerve cells was regulated by the activity of sphingolipid synthetic enzyme including neutral sphingomyelinase 2 (nSMase2 or NSMase2) and sphingomyelin synthase 2 (SMS2). In a transwell experimentation, the upregulation of exosome secretion by the treatment of nerve cells in SMS2 siRNA promoted the intake of Aβ into microglia cells, and significantly reduced the extracellular Aβ level. The finding by the inventors indicates a new mechanism which assumes the clearance of Aβ through the binding of the Aβ to the exosome. The regulation of the release and/or the removal of vesicle may change the risk of AD.

As such, with regard to the present invention, a neural disease such as Alzheimer's disease, or the condition, disorder or disease associated with amyloid β (Aβ) is known to occur due to multiple factors. Accordingly, the actualization of a medicament having a novel working mechanism may supplement weak points of existing medicaments.

The inventors controlled the metabolism of sphingolipid, thereby developed a method for ameliorating a condition, disorder or disease associated with amyloid β, such as Alzheimer's disease, using a working mechanism that had not existed before.

The inventors proposed a method for ameliorating a condition, disorder or disease associated with amyloid β, such as Alzheimer's disease, by controlling a sphingomyelin synthetic enzyme, such as synthetic enzyme SMS2 of sphingomyelin which is a type of sphingolipid. The SMS produces an equimolar amount of ceramide when synthesizing sphingomyelin. Herein, the association with exosome is shown. Further, herein, the association is shown with phosphatidylserine-specific modulation of a neural disease such as Alzheimer's disease. In addition, herein, the association is shown between the aggregation of amyloid β and exosome. In addition, herein, the association is shown between SMS (sphingomyelinase synthetic enzyme) and SMS2, and a neural disease such as Alzheimer's disease. Thus, the present invention provides a novel screening method related to a condition, disorder or disease associated with amyloid β.

Accordingly, the inventors thought that a prevention or treatment effect could be expected for a neural disease such as Alzheimer's disease by inhibiting the SMS2 function, and considered the application to the treatment with, for example, an RNAi (RNA interference:RNA interference) method, which is a novel molecule-specific knockdown method.

An RNAi method is a technique of using a short interference dsRNA (siRNA (small interfering RNA)) to suppress the expression of a specific gene. The RNAi is a phenomenon found by Fire et al., in 1998 (Fire et al., Nature. 391: 806-11, (1998)), where a duplex RNA (double strand RNA) strongly suppresses an expression of a homologous target gene. This has been drawing the attention because it is much simpler compared to conventional congenic methods using a vector or the like, it has high specificity to a target, and it can be applied to genetic treatment. Among molecules that mediate RNAi, a small interfering RNA (siRNA) is in advancement in terms of its application (Elbashir et al., Nature. 411: 494-8, (2001)). It was found that a plurality of sequences to be a target was selected from the mRNA sequence of SMS2, the siRNA thereof was synthesized, and the SMS2 was knocked down, which would be linked to the curing of diseases.

### Advantageous Effects of Invention

Conventionally, there does not exist any medicament for ameliorating a disease associated with amyloid β (Aβ), such as Alzheimer's disease, or a neural disease, via synthetic control of sphingolipid. Thus, the present invention enables the manufacture of a medicament that has a novel working mechanism.

### BRIEF DESCRIPTION OF DRAWINGS

FIG. 1 shows Aβ amyloid generation by an exosome derived from a nerve cell. A; an exosome was collected from a culture supernatant of neuroblastoma N2a, through gradual centrifugation, as shown in the Examples. 100,000×g pellets were loaded, which were subjected to sucrose gradient centrifugation. Thus obtained fractions were analyzed with regard to exosome proteins, such as Alix and Tsg101, as well as GM1 ganglioside. B; a purified exosome (100,000×g pellet) was negatively dyed with phosphotungstic acid, followed by observation through an electron microscope. A scale bar is such that the right side is 500 nm while the left side is 100 nm. C; a culture medium of N2a cells was subjected to gradual centrifugation. Thus obtained pellets, 3,000×g (P3), 4,000×g (P4), 10,000×g (P10) and 100,000×g (P100), were mixed with 25 μM soluble Aβ, followed by incubation at 37° C. for 24 hours. The amyloid fibril formed in the incubation mixture was assayed through thioflavin T (ThT) assay. The value was ±SEM (n=3) on average. D; throughout the indicated time, ThT fluorescence intensity was assayed in the mixture which contained 25 μM Aβ and which contained or did not contain the exosome derived from a culture supernatant of N2a cells or cortex nerve cells. The value is shown as average ±SEM. *p<0.05, **p<0.01, ***p<0.001; t test.

FIG. 2 shows an influence of exosome on the oligomer formation and toxicity of Aβ. A; purified N2a-derived exosome was mixed with 25 μM soluble Aβ1-42, followed by incubation at 37° C. for 0 hours, 1 hour, 3 hours, 5 hours, 10 hours and 24 hours. This incubation mixture was subjected to a dot-blot assay using an anti-oligomer antibody (A11) and an anti-Aβ antibody (6E10). B; Aβ1-42 (25 μM) was incubated at 37° C. for 5 hours, together with N2a-derived exosome, or without the N2a-derived exosome. This incubation mixture was next exposed to a cortex nerve cell for 24 hours. The cell survival rate was assayed using WST-1 assay. The data are represented as average ±SEM. **p<0.01, ***p<0.001; t test.

FIG. 3 shows a condition of sphingolipid metabolism to exosome secretion and Aβ amyloid generation. A-B; N2a cells or cortex nerve cells were treated with imipramine, GW4869, D609 or a vesicle thereof for 24 hours. Next, an exosome was collected from a culture medium, followed by SDS-PAGE, and then western blot, thus detecting Alix, Tsg101 and GM1ganglioside. The pellet of the exosome was purified from a culture of 5×10⁶ cells. (A) Representative blot of Alix in N2a cell lysate and 100,000×g pellets (exosome). The cell lysate was purified from 2.5×10⁵ cells (B) Quantitative analysis related to western blot. The result is represented as average ±SEM. *p<0.05, **p<0.01; t test. C-D; small interfering RNA (siRNA) to aSMase, nSMase1, nSMase2, SMS1 and SMS2 was delivered to the cells in N2a. The exosome was purified from a medium of cells on which siRNA treatment was performed, and the amount of the exosome marker was assayed by western blot. (C) Alix was detected in the cell lysate and exosome. (D) The band strength of the exosome marker was analyzed. The data is shown as average ±SEM. *p<0.05, **p<0.01; t test. E; N2a cells were treated with C6-ceramide (50 μM) or bacterial SMase (100 μU)/ml) for 24 hours. The release level of the exosome was evaluated by western blot. The result is shown as average ±SEM. *p<0.05; t test. F; the exosome was isolated from a culture of N2a cells or primary cultured nerve cells which were treated with the indicated reagent. Next, they were incubated, together with 25 μM soluble Aβ42, at 37° C. throughout the indicated time. The Aβ amyloid fibril formed in the mixture was assayed using ThT assay. The data is average ±SEM (n=4). *p<0.05, **p<0.01, ***p<0.001; t test. G; the N2a cells were treated with the indicated siRNA, and then, purified exosome was mixed with 25 μM Aβ42. After 5 hour incubation, ThT fluorescence was assayed. The data is shown as average ±SEM (n=4). *p<0.05; t test.

FIG. 4 shows the movement of exosome into microglia. A; the exosome which was purified from an N2a cell culture was labeled with a dye PKH26 (red dye), followed by adding it to a microglia cell line BV-2, or a primary culture of microglia or cortex nerve cells. After incubation with the exosome for 3 hours, the cells were immobilized, DAPI-dyed, and analyzed with a confocal microscope. B; a N2a-derived exosome was allowed to bind to an AlexaFluor-conjugated annexin V (AV) and cholera toxin subunit B (CTB), and phosphatidylserine (PS) and GMlganglioside (GM1) exposed to the surface were detected respectively. A fluorescent label was visualized through a confocal microscope. The right side panel shows the same view of phase difference (P.C). The scale bar is 200 nm. C-D; the exosome collected from an N2a culture was labeled with a red dye PKH26, and then, AV or CTB treatment was performed, or AV or CTB treatment was not performed. The labeled exosome was added to BV-2 cells, followed by incubation for 3 hours. Thereafter, the cells were immobilized, and dyed with DAPI. (C) confocal images of internalized exosome are shown. (D) fluorescence intensity for each cell was determined by image analyzing. The internalization of the exosome was quantitated from three independent experiments. The value is average ±SEM. ***p<0.001; t test.

FIG. 5 shows acceleration of Aβ intake into microglia by exosome. A-B; N2a-derived exosome was incubated together with 25 μM Aβ1-42, at 37° C. for 5 hours. Next, a pre-incubated mixture was added to BV-2 cells or primary cultured microglia (final concentration of Aβ, 0.5 μM), followed by incubation throughout the indicated time. The level of Aβ1-42 in the BV-2 cells (A) and a conditioned medium (B) was quantified by ELISA. The value is average ±SEM. *p<0.05, **p<0.01, ***p<0.001; t test. C; the N2a-derived exosome, together with 25 μA Aβ1-42, was incubated at 37° C. for 5 hours, and further incubated together with annexin V (AV) or a cholera toxin subunit (CTB) at 37° C. for 15 more minutes. Next, the mixture thereof was added to BV-2 cells or primary cultured microglia, followed by co-culturing for 3 hours. The intracellular level of Aβ was assayed using ELISA. The value is shown as average ±SEM. **p<0.01; t test.

FIG. 6 shows degradation of Aβ within microglia. A; Aβ1-42 (251.1M) was incubated together with N2a-derivd exosome, or without N2a-derived exosome, at 37° C. for 5 hours. Next, such incubation mixture was exposed to BV-2 cells for 3 hours (final concentration of Aβ, 0.5 μM). After washing out free Aβ and exosome in the medium, the cells were further tracked up to 48 hours later. At the indicated time points, the intracellular level of Aβ42 was assayed using ELISA. B; the exosome was labeled with PKH26 (red dye), and added to the culture of BV-2 cells. After 3 hour incubation, the cells were dyed with LysoTracker Green, followed by analysis through a confocal microscope. The scale bar is 5 μm. C; the PKH26-labeled exosome was mixed with fluorescent dye FAM-conjugated Aβ42 (25 μM). After 5 hour incubation, such a mixture was exposed to the culture of BV-2 cells for 3 hours, and was dyed with LysoTracker Blue. The scale bar is 5 μm.

FIG. 7 shows promotion of Aβ clearance by SMS2 knockdown. A-C; N2a cells seeded within an insert were transfected by APP770 gene and the siRNA as shown. After 24 hours, the medium was removed and then the insert, to which the N2a cells were attached, was further placed for 24 hours on a well (B) with BV-2 cells, or (A) without BV-2 cells. The Aβ level in the mediums (A, B) and BV-2 cells (C) was assayed using ELISA. The value is average ±SEM. *p<0.05, **p<0.01, ***p<0.001; t test. D; N2a cells were transfected using APP gene, and siRNA with regard to N-SMase2 or SMS2. Mediums were replaced, and incubation was further performed for 24 hours. The exosome was collected from the medium, solubilized with a guanidine HCl buffer, and was subjected to western blot analysis.

FIG. 8 is a schematic view showing a role of exosome in Aβ metabolism. The exosome and Aβ are both generated from nerve cells, and are released to an extracellular space. The secretion of the exosome is bidirectionally regulated by N-SMase2 and SMS2, which are sphingolipid metabolism enzymes. The exosome promotes Aβ amyloid generation on the surface of the exosome, and then, takes part of Aβ fibril into microglia in the form of PS dependency, followed by degradation. There is a possibility for a nerve cellular exosome to promote Aβ clearance.

### DESCRIPTION OF EMBODIMENTS

Hereinafter, preferred embodiments of the present invention will be described. Throughout the present specification, unless specifically referred to, an expression in a singular form is to be understood to encompass the concept of its plurality form. Therefore, unless specifically referred to, singular form articles (for example, “a”, “an”, “the” or the like in English, and corresponding articles and adjectives or the like in other languages) are to be understood to encompass the concept of their plurality form. Furthermore, terms used herein, unless specifically referred to, are to be understood to be used in the meaning usually used in the art. Therefore, unless defined otherwise, all technical terms and scientific terms herein have the same meaning as generally recognized by those skilled in the art to which the present invention belongs. If they contradict, the present specification (including the definition) governs.

### DEFINITION

Herein, the following abbreviations will be used as needed.
Aβ: amyloid β (βamyloid) (protein)
SM: sphingomyelin
SMS: sphingomyelin synthetic enzyme
SMS1, SMS2, N-SMase2: gene name
5. SMase: sphingomyelinase
KO: knock out
wKO: double knock out
MEF: murine embryonic fibroblast
FBS: fetal bovine calf serum
DMEM: Dulbecco Modified Eagle medium
WT: wild type

Hereinafter, definitions will be listed for the terms particularly used in the specification.

As used herein, “sphingomyelin synthetase” (also referred herein as “SMS”) refers to an enzyme that synthesizes sphingomyelin (also referred herein as “SM”) wherein it converts ceramide into sphingomyelin in the presence of phosphatidylcholine (also referred herein as “PC”), and which plays an important role in cell death and survival (see, Non-patent Literatures 14 and 15). Here, phosphatidylcholine after conversion is converted into diacylglycerol. As sphingomyelin synthetases, typically, SMS1 and SMS2 are known, as well as homologs (see, Non-patent Literatures 14 and 15). SMS1 is known to be expressed in the Golgi body and be involved in de novo synthesis of SM. On the other hand, SMS2 is expressed in the Golgi body and cell membrane, and its physiological function is not known in detail (see, Non-patent Literature 16). In addition, GenBank Accession numbers of SMS1 are NM_{—}147156 (human) and NM_{—}144792 (mouse), and those of SMS2 are BC028705.1 (human), BC041369.2 (human) and NM_{—}028943 (mouse).

As used herein, “condition, disorder or disease associated with amyloid β” refers to any condition, disorder or disease associated with the behavior of amyloid β. Such a condition, disorder or disease includes, without limitation, dementia such as Alzheimer's disease as well as retinal disease and the like. The retinal disease includes glaucoma, diabetic retinopathy, age-related macular degeneration (age-related macular degeneration: AND), retinitis pigmentosa, and the like. As described in Journal of Pharmacological Sciences, Vol. 134 (2009), No. 6, 309-314 and the like, the association with the present invention includes, in particular, glaucoma and age-related macular degeneration, without limitation.

As used herein, “dementia” is a condition that used to be referred to as dementedness, and it refers to a condition in which intelligence that is once normally developed is reduced due to an acquired brain organic disorder. The “dementia” is defined as a syndrome accompanied by a cognitive disorder including “memory” and “orientation” in addition to “intelligence” as well as personality disorder.

As used herein, “Alzheimer's disease (Alzheimer's disease (AD)” is used in the same meaning as the meaning used in the subject field. Alzheimer's disease is a type of degenerative dementia, and is a progressive neurodegenerative disease characterized by senile plaque, neurofibrillary tangle, neuronal loss and cognitive disorder. Furthermore, Alzheimer's disease is a type of dementia, primary symptom of which is a decline in cognitive function, and change in personality. From the pathological and biochemical point of view, a constitutive substance of senile plaque, β-amyloid 42 (Aβ42), is assumed to be a causal substance of AD. Alzheimer's disease includes familial Alzheimer's disease (Familial AD; FAD) as well as Alzheimer-type dementia (senile dementia Alzheimer's type (SDAT)). Familial Alzheimer's disease indicates a complete autosomal dominant inheritance, and is also referred to as hereditary Alzheimer's disease. On the other hand, Alzheimer-type dementia refers to the Alzheimer's disease that develops at old age (60 years old and more), which accounts for most cases of Alzheimer's disease. In the present invention, it is understood that both types of Alzheimer's diseases are intended.

As used herein, “sphingomyelinase (SMase)” refers to an enzyme which hydrolyzes sphingomyelin (SM). Through the degradation of sphingomyelin by SMase, ceramide and phosphorylcholine are generated. In the present invention, “neutral sphingomyelinase 2(N-SMase2; also represented as nSMase2)” is a type of SMase, which refers to those having an optimum pH condition in neutrality, and which is also referred to as sphingomyelinphosphodiesterase 3. The GenBank Accession numbers of nSMase2 are NM_{—}018667 (human), NM_{—}021491 (mouse), and NM_{—}053605 (rat).

As used herein, “microglia” is also referred to as “microgliocyte”, and refers to a small neuroglial cell in the central nervous system. The microglia has many protruding portions, and it moves in an ameboid manner in a pathological condition, and demonstrates a phagocytic function. Small neuroglial cells include oligodendrocytes (oligodendroglial cell) with a small cell body and with a small number of protrusions, and Hortega cells (Hortega's cell) with a small cell body, rich in branching, and which actively ingests foreign materials. The former performs myelin formation in the axon of the central nervous system. The latter has a different origin from the other neuroglial cells, and is defined as a mesoderm. Accordingly, the latter is referred to as a mesoglial cell, and is also viewed as another species from the other neuroglial cells. Microgliocyte (microglial cell) is used as a different name for the latter. Note that neurilemma is regarded as a matter of the same nature in the peripheral nervous system.

As used herein, “exosome” is used as defined to be normally used in the subject field, and refers to an extracellular vesicular granule of 30 to 100 nanometers in diameter. The exosome is considered such that any cells are secreted therefrom.

As used herein, “amyloid βprotein (Aβ)” is used as defined to be normally used in the subject field, and it typically has a partial sequence of SEQ ID NO: 89 (human amyloid β (1-55)=DAEFRHDSGY EVHHQKLVFF AEDVGSNKGA IIGLMVGGVVIATVIVITLV MLKKK). Herein, there are many types of Aβ, and thus it will be shown as Aβ (starting point)-(ending point), such as Aβ11-42, Aβ17-42. Those starting withposition 1 may also be represented as Aβ (number) based on the number of amino acids, and these numbers may also be represented as a subscript. For example, Aβ42 (or Aβ1-42) consists of amino acid numbers 1 to 42 of SEQ ID NO: 89, and Aβ40 (or Aβ1-40) consists of amino acid numbers 1 to 40 of SEQ ID NO: 89.

As used herein, “phosphatidylserine (PS)” is used as defined to be normally used in the subject field, and it refers to a phospholipid in which a polar group is serine.

As used herein, “ceramide” is used as defined to be normally used in the subject field, and is defined as one of the acids obtained by coupling sphingosine with a fatty acid.

As used herein, “expression” of a gene, polynucleotide, polypeptide and the like refers to the gene or the like being subjected to a certain action in vivo to confer a different form. Preferably, it refers to a gene, polynucleotide and the like being transcribed and translated to be in a form of polypeptide; however, it may also be one mode of expression for a gene, polynucleotide and the like to be transcribed so that mRNA is created. More preferably, the form of such a polypeptide may be the one that has been subjected to post-translation processing.

Accordingly, as used herein, “decrease” in the “expression” of a gene, polynucleotide, polypeptide and the like refers to a state where the amount of expression significantly decreases when the factor according to the present invention is allowed to work, compared to when the factor is not allowed to work. Preferably, the decrease in the expression includes a decrease in the expression amount of polypeptide. As used herein, “increase” in the “expression” of a gene, polynucleotide, polypeptide and the like refers to a state where the amount of expression significantly increases when the factor according to the present invention is allowed to work, compared to when the factor is not allowed to work. Preferably, the increase in expression includes the increase in the expression amount of polypeptide. As used herein, “induction” of the “expression” of a gene refers to a concept of allowing a certain factor to work on a certain cell to increase the expression amount of the gene. Accordingly, the induction of expression encompasses a concept of, when the expression of a certain gene is not recognized at all, allowing the gene to express, and a concept of increase in the expression of a certain gene when the expression of the gene has already been recognized.

As used herein, “detection” or “quantification” of a gene expression (e.g., mRNA expression, and polypeptide expression) may be achieved by using, for example, an appropriate method, including assay and immunoassay methods of mRNA. As for molecular biological methods, for example, northern blotting, dot blotting and PCR method are exemplified. As for immunoassay methods, for example, ELISA, RIA, a fluorescence antibody method, western blotting, an immune structure dyeing method and the like using a microtiter plate are exemplified as the methods. Furthermore, as for a quantitative method, ELISA and RIA are exemplified. The detection or quantification may also be performed by a gene analysis method using an array (e.g., DNA array, and protein array). As for the DNA array, “DNA microarray to saishin PCR hou [DNA microarray and latest PCR method]” (Shujunsha Co., Ltd, Saibou Kougaku Bessatsu [Cell Technology, Additional Volume]) widely provides general information. As for the protein array, it is detailed in Nat Genet 2002 December; 32 Suppl: 526-32. As for a method for analyzing gene expression, in addition to the above-mentioned matters, it includes, without limitation, RT-PCR, RACE method, SSCP method, immunoprecipitation method, two-hybrid system, in vitro translation and the like. Such a method for further analysis is described in, for example, genome kaiseki jikkenhou, Nakamura Yusuke lab manual [Genome analysis experimental method, Nakamura Yusuke lab manual] “edited by Nakamura Yusuke, Yodosha Co., Ltd (2002), and the like. All of the descriptions thereof are incorporated by reference herein.

As used herein, “expression amount” refers to an amount in which a polypeptide or mRNA is expressed in a cell of interest or the like. Such expression amount includes expression amount of the polypeptide of the present invention at a protein level evaluated by any suitable methods using the antibody of the present invention including immunological measuring methods such as ELISA method, RIA method, fluorescent antibody method, Western blot method, immunohistological staining method or the like, or expression amount of the polypeptide of the present invention at an mRNA level evaluated by any suitable method including molecular biological measuring methods such as Northern blot method, dot blot method, PCR method or the like. “Change of expression amount” means increase or decrease in expression amount of a polypeptide of present invention at a protein level or mRNA level evaluated by any suitable method including the above immunological measuring methods or molecular biological measuring methods.

As used herein, it is understood that “protein” of “N-SMase2” may be a functionally equivalent variant or derivative thereof, as long as it has the functions of N-SMase2, in addition to a sequence set forth by NM_{—}018667 (human), NM_{—}021491 (mouse), and NM_{—}053605 (rat).

As for an embodiment of an aspect of “N-SMase2” according to the present invention, exemplified are: a polypeptide set forth by an amino acid sequence selected from the group consisting of SEQ ID NOs: 84, 86 and the like; a polynucleotide encoding a polypeptide set forth by an amino acid sequence selected from the group consisting of SEQ ID NOs: 84, 86 and the like; or a vector (e.g., expression vector) comprising a polynucleotide set forth by a base sequence selected from the group consisting of SEQ ID NOs: 83, 85 and the like, any of which is used from the viewpoint of promoting a condition, disorder or disease associated with amyloid β. As for the polypeptide according to the present invention, exemplified are: a polypeptide consisting of an amino acid sequence of SEQ ID NOs: 84, 86 and the like; a polypeptide consisting of an amino acid sequence, in which one or several amino acids are deleted, substituted or added in the above-mentioned amino acid sequence, and which has a disease associated with sphingomyelinase activity or amyloid β; and a derivative thereof, a partial peptide as well as a salt thereof.

As used herein, “derivative of polypeptide” refers to, for example, a derivative obtained by the acetylation, palmitoylation, myristylation, amidation, acylation, dansylation, biotinylation, phosphorylation, succinylation, anilidation, benzyloxycarbonylation, formylation, nitration, sulphonation, aldehydation, circularization, glycosylation, monomethylation, dimethylation, trimethylation, guanidinylation, amidination, maleylation, trifluoroacetylation, carbamoylation, trinitrophenylation, nitrotroponylation, polyethylene-glycolation or acetoacetylation, or the like of a polypeptide. Among these, acetylation of the N-terminus, amidation of the C-terminus, and methylation of the C-terminus add resistibility to exopeptidase which degrades a polypeptide from the terminus. Also, the stability is expected to be high in living organisms by glycosylation or polyethylene-glycolation, it is preferable when the subject effect is desired.

Variants of the protein which can be used in the present invention also include the one having “an amino acid sequence in which one or several amino acids are deleted, substituted or added” of the protein consisting of the above-mentioned sequence. For example, a protein is included which comprises (A) an amino acid sequence in which one or two or more (preferably, about 1 to 30, preferably about 1 to 10, even more preferably several, such as 1 to 5) amino acids are deleted in an amino acid sequence of SEQ ID NO: 84, 86 or the like, (B) an amino acid sequence in which one or two or more (preferably, about 1 to 30, preferably, about 1 to 10, even more preferably several, such as 1 to 5) amino acid sequences are substituted with other amino acids in an amino acid sequence of SEQ ID NO: 84, 86 or the like, (C) an amino acid sequence in which one or two or more (preferably, about 1 to 30, preferably, about 1 to 10, even more preferably several, such as 1 to 5) amino acids are added to an amino acid sequence of SEQ ID NO: 84, 86 or the like, or (D) an amino acid sequence in combination thereof. When an amino acid sequence is deleted, substituted, or added, the position of the deletion, substitution or addition is not particularly limited. However, the protein used in the present invention is a polypeptide which can treat or prevent a condition, disorder or disease associated with sphingomyelinase or amyloid β even after the deletion, substitution or addition. Such a polypeptide includes a polypeptide having at least 60% or more homology with, preferably a polypeptide having 80% or more homology with, even more preferably, having 90% or more, or 95% or more homology with an amino acid sequence of SEQ ID NO: 84, 86 or the like.

The “partial peptide of protein” which can be used in the present invention may be any partial peptide which is a partial peptide of said protein of the present invention, and preferably, which has characteristics similar to those of said protein of the present invention. For example, a peptide or the like is used which has at least 20 or more, preferably 50 or more, even more preferably 70 or more, more preferably 100 or more, and most preferably 200 or more, amino acid sequences among the constituent amino acid sequences of the protein according to the present invention.

In addition, with regard to the “partial peptide” which may be used in the present invention, as long as it treats or prevents a condition, disorder or disease associated with sphingomyelinase or amyloid β, one or two or more (preferably, about 1 to 10, and even more preferably several, such as 1 to 5) amino acids may be deleted in the amino acid sequence thereof, or one or two or more (preferably, about 1 to 20, more preferably, about 1 to 10, and even more preferably several, such as 1 to 5) amino acids may be substituted in the amino acid sequence thereof, or one or two or more (preferably, about 1 to 20, more preferably, about 1 to 10, and even more preferably several, such as 1 to 5) amino acids may be added in the amino acid sequence thereof. Such a polypeptide includes a polypeptide having at least 60% or more homology with, preferably a polypeptide having 80% or more homology with, even more preferably, having 90% or more, or 95% or more homology with an amino acid sequence of SEQ ID NO: 84, 86 or the like.

As used herein, “salt” refers to any salt of polypeptide or a derivative thereof, and preferably, any pharmaceutically acceptable salt (including inorganic salt and organic salt). For example, included are sodium salt, potassium salt, calcium salt, magnesium salt, ammonium salt, hydrochloride salt, hydrosulfate, nitrate salt, phosphate, organic acid salt (acetate salt, citric salt, maleate, malate, oxalate, lactate salt, succinate, fumarate, propionate, formate, benzoate, picrate, benzene sulfonate and the like) and the like of a polypeptide or a derivative thereof.

The derivative of the above-mentioned polypeptide may be produced using a method publicly known in the subject field. In addition, the salt of the above-mentioned polypeptide may be easily produced by those skilled in the art using any method publicly known in the subject field.

With regard to the polynucleotide used for the “expression vector” of “N-SMase2” in the present invention, exemplified are: a polynucleotide consisting of a base sequence of SEQ ID NO: 83, 85 or the like; and a polynucleotide which can hybridize under a stringent condition with the above-mentioned polynucleotide or a complementary strand thereof, the polynucleotide encoding a polypeptide having a disease associated with sphingomyelinase activity or amyloid β.

The “polynucleotide which can hybridize under a stringent condition” as used herein means a polynucleotide obtained by a well-known and commonly-practiced method in the subject field, such as colony hybridization method, plaque hybridization method, or southern blot hybridization method, with a fragment of a certain polynucleotide as a probe. Specifically, the polynucleotide means such a polynucleotide which can be identified by performing hybridization, for example, under the presence of 0.7 to 1.0 M NaCl at 65° C., using a membrane in which a colony or plaque-derived polynucleotide is immobilized, and by washing the membrane at 65° C. using a SSC (Saline Sodium Citrate: 150 mM sodium chloride, 15 mM sodium citrate) solution of 0.1 to 2 times concentration. The hybridization can be performed in accordance with a method described in Molecular Cloning: A Laboratory Manual, Second Edition (1989) (Cold Spring Harbor Laboratory Press), Current Protocols in Molecular Biology (1994) (Wiley-Interscience), DNA Cloning 1: A Practical Approach Core Techniques, Second Edition (1995) (Oxford University Press) or the like. Herein, preferably, a sequence consisting only of adenine (A) or thymine (T) is excluded from sequences that hybridize under a stringent condition.

As used herein, “hybridizable polynucleotide” refers to a polynucleotide which can hybridize to another polynucleotide under the above-mentioned hybridization condition. Specifically, for example, with regard to a polynucleotide set forth by a base sequence of SEQ ID NO: 83, 85, 89, 90 or the like, such a polynucleotide includes a polynucleotide which has at least 60% or more, preferably 80% or more, and more preferably 95% or more homology with a polynucleotide set forth by a base sequence of SEQ ID NO: 83, 85, 89, 90 or the like. Herein, as for the homology, degree of similarity is shown in score by using, for example, BLAST, a search program using the algorithm developed by Altschul et al., (The Journal of Molecular Biology, 215, 403-410 (1990)).

The above polynucleotide can be prepared in accordance with a publicly known method. The above polynucleotide can also be prepared, based on the amino acid sequence, by chemically synthesizing the polypeptide or DNA encoding the polypeptide. The chemical synthesis of DNA can be performed by using a DNA synthesizer of Shimadzu Corporation, which utilizes a thiophosphite method, a DNA synthesizer of Applied Biosystems, which utilizes a phosphoamidite method, or the like.

As used herein, “expression vector” refers to a nucleic acid sequence in which various regulatory elements or control sequences can operate in a cell of a host in addition to a structural gene (e.g., intended N-SMase2, for example) and a promoter which regulates the expression thereof. The regulatory element can preferably comprise a terminator, a selection marker such as a drug resistance gene, and an enhancer. It is a well known fact for those skilled in the art that the type of expression vector of living organisms (e.g., mouse, human and the like) and the type of regulatory element to be used may vary in accordance with host cells. The term “control sequence” as used herein refers to a DNA sequence having a functional promoter and any associated transcription element (e.g., enhancer, TATA box and the like).

As used herein, “operably linked” refers to a state in which a polynucleotide related to a gene, various regulatory elements such as a promoter or enhancer for regulating the expression of the gene, are linked in an operative manner in a host cell so that the gene can be expressed. In a case where the term refers to a transcription regulatory sequence or a translation regulatory sequence, such as a promoter, the term refers to a state in which a desired expression (working) of a sequence is arranged under the control of the transcription regulatory sequence or the translation regulatory sequence.

As used herein, “promoter” refers to a region on a DNA, where an initiation site is determined for the transcription of a gene and where the frequency thereof is directly regulated; and it is a base sequence in which an RNA polymerase binds and initiates transcription. While an estimated promoter region varies in accordance with a structural gene, it is usually at the upstream of a structural gene. However, without limitation, the estimated promoter region may also be at the downstream of the structural gene. In the present invention, a single promoter may be used, and a plurality of promoters may also be used.

While the virus vector used in the present invention can be created based on a DNA or RNA virus, the virus species of the origin is not particularly limited, and it can be any virus vector such as MoMLV vector, herpes virus vector, adenovirus vector, adeno-associated viral vector, HIV vector, Sendai virus vector, and vaccinial virus vector.

The “retrovirus”, which may be used in the present invention, is a single-chain and diploid RNA virus, which proliferates via a virion of a retrovirus and reverse transcriptase enzyme. The retrovirus may be replication-competent or may not be replication-competent. The term “retrovirus” refers to any of the publicly known retroviruses (type-c retrovirus such as Moloney murine leukemia virus (MoMuLV), Harvey murine sarcoma virus (HaMuSV) murine mammary tumor virus (MuMTV), gibbon ape leukemia virus (GaLV), feline leukemia virus (FLV) and avian sarcoma virus (RSV) and the like). The “retrovirus” which may be used in the present invention also includes human T cell leukemia virus (HTLV-1 and HTLV-2), and lentiviru families of retrovirus (without limitation, human immunodeficiency viruses HIV-1 and HIV-2, simian immunodeficiency virus (SIV), feline immunodeficiency virus (FIV), and equine immunodeficiency virus (EIV) and the like).

As a naked DNA form, the form of a plasmid DNA can be preferably exemplified, and the plasmid includes a publicly known, animal cell expressing vector plasmid. The vector plasmid preferably includes a virus promoter, such as CMV (cytomegalovirus) promoter, RSV (Rous sarcoma virus) promoter, promoter of HSV-1 virus TK gene, SV40 (simian virus 40) early promoter, and adenovirus MLP (major late promoter) promoter. In addition, it is also possible to include a marker gene capable of selecting or identifying a transfected cell; and the marker gene includes a neo gene which provides a resistance characteristic to an antibiotic substance G418 (encoding neomycinphosphotransferase), dhfr (dihydrofolate reductase) gene, CAT (chloramphenicol acetyl transferase) gene, pac (puromycin acetyltransferase) gene, and gpt (xanthine guanine phosphoribosyl transferase) gene.

As used herein, “substance (e.g., nucleic acid) for suppressing expression (of a gene such as SMS2)” is not particularly limited as long as it is a substance for suppressing the transcription of mRNA of a target gene, a substance for degrading the transcribed mRNA (such as nucleic acid), or a substance for suppressing the translation of protein from mRNA (e.g., nucleic acid). As for the subject substance, exemplified are nucleic acids such as siRNA, antisense oligonucleotide, ribozyme or an expression vector thereof. Among them, siRNA and an expression vector thereof are preferable, and in particular, siRNA is preferable. “Substance for suppressing the expression of gene” includes, besides the ones described above, protein, peptide, and other small molecules. Note that, in one embodiment, the target gene according to the present invention is a. SMS2 gene.

As used herein, “siRNA” refers to an RNA molecule having a duplex RNA portion consisting of 15 to 40 bases, and the siRNA has a function of cleaving mRNA of a target gene which has a complementary sequence with an antisense strand of said siRNA, and of suppressing the expression of the target gene. In detail, the siRNA according to the present invention is an RNA which includes a duplex RNA portion consisting of a sense RNA strand consisting of a sequence homologous to a contiguous RNA sequence in the mRNA of SMS2 gene, and an antisense RNA strand consisting of a sequence complementary to the sense RNA sequence. The designing and manufacturing of the subject siRNA and a mutant siRNA to be described below are within the scope of the technique of those skilled in the art.

The length of the duplex RNA portion is, as a base sequence, 15 to 40 bases, preferably 15 to 30 bases, more preferably 15 to 25 bases, still preferably 18 to 23 bases, and most preferably 19 to 21 bases. It is understood that the upper and lower limits thereof are not limited to the specified ones, and it may be any combination of the ones listed above. As to the terminal structure of the sense strand or antisense strand of siRNA, there is no particular restriction, and it may be selected as appropriate in accordance with purposes. For example, the terminal structure may be that having a blunt end, and may be that having a protruding end (overhang); and the type in which the 3′ terminus is protruded is preferable. The siRNA, having an overhang consisting of several bases, preferably 1 to 3, and more preferably 2, at the 3′ terminus of the sense RNA strand and antisense RNA strand, often has an effect of suppressing the expression of a target gene, which is preferable. There is no particular restriction on the type of the bases of the overhang, and it may be either a base that constitutes the RNA or a base that constitutes the DNA. A preferable overhang sequence includes dTdT (2 bp deoxy T) and the like at the 3′ terminus. For example, preferable siRNA includes, without limitation, those in which dTdT (2 bp deoxy T) is added to the 3′ terminus of the sense or antisense strand of all the siRNA.

Further, it is also possible to use an siRNA in which one to several nucleotides are deleted, substituted, inserted and/or added at either or both of the sense strand and antisense strand of the above-mentioned siRNA. Herein, the term, one to several bases, is not particularly limited; however, it refers to preferably 1 to 4 bases, even more preferably 1 to 3 bases, and most preferably 1 to 2 bases. The specific examples of the subject mutation includes, without limitation, those with the base number at the overhand portion is 0 to 3, those in which the base sequence of the 3′-overhang portion is changed to another base sequence, those in which the length of the above-mentioned sense RNA strand and antisense RNA strand is different by 1 to 3 bases due to the insertion, addition or deletion of bases, those in which the base in the sense strand and/or antisense strand is substituted with another base, and the like. However, it is necessary for the sense strand and antisense strand to hybridize in these mutant siRNAs, and it is necessary for these mutant siRNAs to have a gene expression inhibitory capability equivalent to that of siRNAs that do not have mutations.

Further, the siRNA may also be an siRNA (Short Hairpin RNA; shRNA) that has a molecule of a structure in which one of the ends is closed, such as a hairpin structure. The shRNA is an RNA comprising a sense strand RNA of a specific sequence of a target gene, an antisense strand RNA consisting of a sequence complementary to the sense strand sequence, and a linker sequence for connecting both strands thereof; and the sense strand portion hybridizes with the antisense strand portion to form a duplex RNA portion.

It is desirable for the siRNA not to show a so-called off-target effect when clinically used. The off-target effect refers to an action for suppressing the expression of another gene which is partially homologous to the siRNA used, other than the target gene. In order to avoid the off-target effect, it is possible to confirm that a candidate siRNA does not have cross reactivity by using DNA microarray in advance. Furthermore, it is also possible to confirm as to whether there is no gene that includes a portion having high homology with a sequence of a candidate siRNA, other than a target gene, using a publicly known database provided by NCBI (National Center for Biotechnology Information) or the like, to avoid the off-target effect.

It is possible to use publicly known methods, such as a method using chemical synthesis, and a method using genetic recombination technology, to create the siRNA according to the present invention. In the method using the synthesis, it is possible to synthesize a duplex RNA based on sequence information using an ordinary method. Furthermore, in the method using genetic recombination technology, it is possible to create siRNA by constructing an expression vector encoding a sense strand sequence and an antisense strand sequence, introducing the vector into a host cell, and obtaining each of the sense strand RNA and antisense strand RNA that was generated by transcription. Furthermore, it is also possible to create a desired duplex RNA by expressing shRNA that comprises: a sense strand of a specific sequence of a target gene, an antisense strand consisting of a sequence complementary to the sense strand sequence, and a linker sequence for connecting the both strands thereof, and that forms a hairpin structure.

With regard to the siRNA, as long as it has expression inhibitory activity of the target gene, all or part of the nucleic acid constituting siRNA may be a natural nucleic acid, or a modified nucleic acid.

For the nucleic acid for suppressing the expression of genes such as SMS2 of the present invention, a modified nucleic acid may be used. A modified nucleic acid means that in which a modification is provided at nucleoside (base portion, sugar portion) and/or a binding portion between nucleosides, and which has a structure different from that of natural nucleic acids. “Modified nucleoside” constituting a modified nucleic acid includes, for example, abasic nucleoside; arabino nucleoside, 2′-deoxyuridine, α-deoxyribo nucleoside, β-L-deoxyribo nucleoside, and other glycosylated nucleoside; peptide nucleic acid (PNA), peptide nucleic acid to which a phosphate group is bound (PHONA), locked nucleic acid (LNA), morpholino nucleic acid, and the like. The nucleoside having the above-mentioned glycosylation includes a nucleoside having sugar modification of substituted pentasaccharide such as 2′-O-methylribose, 2′-deoxy-2′-fluororibose, 3′-O-methylribose and the like; 1′,2′-deoxyribose; arabinose; substituted arabinose sugar; hexasaccharide and alpha-anomer. These nucleosides may also be a modified base in which the base portion is modified. Such a modified base includes, for example, pyrimidine such as 5-hydroxycytosine, 5-fluorouracil, 4-thiouracil and the like; purine such as 6-methyladenine, 6-thioguanosine and the like; and other heterocyclic bases.

“Binding between modified nucleoside” constituting a modified nucleic acid includes, for example, alkyl linker, glycelyl linker, amino linker, poly (ethylene glycol) binding, binding between methyl phosphonate nucleosides; methylphosphonothioate, phosphotriester, phosphothiotriester, phosphorothioate, phosphorodithioate, triester prodrug, sulfone, sulfonamide, sulfamate, holm acetal, N-methylhydroxylamine, carbonate, carbamate, morpholino, boranophosphonate, phosphoramidate and other binding between non-natural nucleosides.

As for a nucleic acid sequence included in the duplex siRNA according to the present invention, it is possible to use the sequences described in the sequence listing in a preferable manner. Table 1 shows nucleotide sequences of these siRNAs. In Table 1, those shown in upper case letters are sense RNA sequences and antisense RNA sequences; and those shown in lower case letters or d+upper-case letter (which means deoxy body) are 3′ terminal overhang sequences.

TABLE 1

| siRNA | | |
|---|---|---|
| Name | Sequences | |
| SMS2-i1 | GGUCACUUGGAAAGUCAAA-dTdT | (SEQ ID NO: 53) |
| | dTdT-CCAGUGAACCUUUCAGUUU | (SEQ ID NO: 54) |
| SMS2-i2 | CCGGACUACAUCCAGAUUU-dTdT | (SEQ ID NO: 55) |
| | dTdT-GGCCUGAUGUAGGUCUAAA | (SEQ ID NO: 56) |
| SMS2-i3 | GGAUGGUAUUGGUUGGGUU-dTdT | (SEQ ID NO: 57) |
| | dTdT-CCUACCAUAACCAACCCAA | (SEQ ID NO: 58) |
| SMS2-i4 | GCAGAUUGUUGUUGAUCAU-dTdT | (SEQ ID NO: 59) |
| | dTdT-CGUCUAACAACAACUAGUA | (SEQ ID NO: 60) |
| SMS2-i5 | CAUAGAGACAGCAAAACUU-dTdT | (SEQ ID NO: 61) |
| | dTdT-GUAUCUCUGUCGUUUUGAA | (SEQ ID NO: 62) |
| SMS2-i6 | GCAUUUUCUGUAUCAGAAA-dTdT | (SEQ ID NO: 63) |
| | dTdT-CGUAAAAGACAUAGUCUUU | (SEQ ID NO: 64) |
| SMS2-i7 | GUCACUUCUGGUGGUAUCA-dTdT | (SEQ ID NO: 65) |
| | dTdT-CAGUGAAGACCACCAUAGU | (SEQ ID NO: 66) |
| SMS2-i8 | CUGUUUUGGUGGUACCAUU-dTdT | (SEQ ID NO: 67) |
| | dTdT-GACAAAACCACCAUGGUAA | (SEQ ID NO: 68) |
| SMS2-i11 | GGCUCUUUCUGCGUUACAA-dTdT | (SEQ ID NO: 69) |
| | dTdT-CCGAGAAAGACGCAAUGUU | (SEQ ID NO: 70) |
| SMS2-i104 | GGGCAUUGCCUUCAUAUAU-dTdT | (SEQ ID NO: 71) |
| | dTdT-CCCGUAACGGAAGUAUAUA | (SEQ ID NO: 72) |
| SMS2-i105 | GGCUGUUUCUGAGAUACAA-dTdT | (SEQ ID NO: 73) |
| | dTdT-CCGACAAAGACUCUAUGUU | (SEQ ID NO: 74) |
| SMS2-i106 | GGUGGUGGAUUGUCCAUAA-dTdT | (SEQ ID NO: 75) |
| | dTdT-CCACCACCUAACAGGUAUU | (SEQ ID NO: 76) |
| SMS2-i107 | GGAUUGUCCAUAACUGGAU-dTdT | (SEQ ID NO: 77) |
| | dTdT-CCUAACAGGUAUUGACCUA | (SEQ ID NO: 78) |
| SMS2-i108 | CCAUAACUGGAUCACAUAU-dTdT | (SEQ ID NO: 79) |
| | dTdT-GGUAUUGACCUAGUGUAUA | (SEQ ID NO: 80) |
| SMS2-i109 | GCACACGAACACUACACUA-dTdT | (SEQ ID NO: 81) |
| | dTdT-CGUGUGCUUGUGAUGUGAU | (SEQ ID NO: 82) |

As used herein, “transgenic” refers to integration of a specific gene into an organism (or cell or the like), or an organism (for example, including animals (such as a mouse)) (or cell or the like) wherein such gene is integrated or deleted or suppressed. Among transgenic organisms (or cell or the like), one wherein a gene is deleted or suppressed is referred to as a knockout organism (or cell or the like). Therefore, “knockout”, when referring to animal or cell or the like, means states wherein a native gene which is targeted does not function or is not expressed.

As used herein, “transgenesis” refers to introducing a gene of interest into a cell, tissue or animal, conceptually encompasses “transformation”, “transduction” and “transfection” or the like, and can be realized by any technique known in the art. In addition, “transgenesis” also encompasses any of one wherein a site to be introduced is not limited and one via homologous recombination wherein a site to be introduced is limited. A method for transgenesis include, but not limited to, for example, methods using retrovirus, plasmid, vector or the like, or electroporation method, methods using particle gun (gene gun), calcium phosphate method. A cell used for transgenesis can be any cell, and use of an undifferentiated cell (for example, fibroblast or the like) is preferred.

As used herein, “preventing” refers to, by any means, not allowing the occurrence of or at least delaying a disease, disorder or symptom which the present invention targets before occurrence of the disease, disorder or symptom, or not allowing the occurrence of a disorder even if any cause itself of the disease, disorder or symptom occurs.

As used herein, “treating” refers to stopping the progression of a disease, disorder or symptom which the present invention targets, or completely or partly arresting or ameliorating a disease, disorder or symptom which the present invention targets.

As used herein, “treatment” refers to somewhat affecting a disease, disorder or symptom or preventing a subject from being in such a disease, disorder or symptom, and can encompass therapy and prevention. In narrower sense, “treatment” refers to the above action after occurrence thereof compared to “prevention”.

It is understood that “medicament” herein is interpreted in the broadest sense in the art, comprise any drug and encompass pharmaceuticals under the Parmaceutical Affairs Law, quasi-drugs or the like as well as any usage of drug and composition or the like which is intended for treatment or prevention by osteogenesis. As such examples, applications in medical art, dental art or the like are listed, for example, a gene therapeutic agent or the like is listed. Usually, a medicament comprises a solid or liquid excipient and can optionally comprise additives such as a disintegrating agent, a flavoring agent, a delayed releasing agent, a lubricant, a binding agent, a coloring agent or the like. The form of a medicament includes, but not limited to, tablets, injections, capsules, granules, powders, fine granules, controlled release formulations or the like.

As used herein, “candidate”, “candidate substance” and “test substance” are exchangeablly used, and they refer to a candidate for screening purpose, such as medicament, treatment substance and prevention substance, wherein the candidate is the object of the screening.

As used herein, “contacting” a substance (e.g., test substance and the like) to an object such as a protein and a cell, is used with an ordinary meaning, and it means that the substance and the object are arranged so close with each other that they can interact with each other.

As used herein, “control cell” is a term used for a cell with which a test substance or the like is allowed to contact, and it refers to a cell to which the test substance or the like, used as a control, has not been allowed to contact.

As used herein, “substance for increasing the enzyme activity or expression of the protein of N-SMase2” refers to any substance which increases the enzyme activity or expression of the protein of N-SMase2. Any substance can be such a substance as long as it increases the enzyme activity or expression of the protein of N-SMase2, and it may be a nucleic acid encoding N-SMase2. Without limitation, such a substance includes, for example, a substance for increasing the transcription of mRNA of a target gene, N-SMase2, or a nucleic acid encoding N-SMase2 or an expression vector comprising the nucleic acid, a substance for preventing the mRNA of the transcribed N-SMase2 from being degraded, or a substance for increasing the translation of protein from mRNA of N-SMase2, a substance for preventing translated protein of N-SMase2 from being degraded, a substance for stabilizing translated protein of N-SMase2, a substance for increasing enzyme activity of protein of N-SMase2, a variant of N-SMase2 with increased enzyme activity, a nucleic acid encoding it or an expression vector comprising the nucleic acid, or the like. Furthermore, nucleic acids (naked DNA or expression vector thereof or the like), protein, peptide or other low molecules (e.g., those that can be synthesized by combinatorial chemistry or the like), high molecules, and a complex of these substances and the like are also encompassed.

As used herein, “substance for suppressing enzyme activity or expression of protein of SMS2” refers to any substance for suppressing enzyme activity or expression of protein of SMS2. Any substance can be such a substance as long as it suppresses enzyme activity or expression of protein of SMS2. Without particular limitation, such a substance may be, for example, a substance for suppressing the transcription of a target gene, which is mRNA of SMS2, a substance for degrading transcribed mRNA of SMS2 (such as nucleic acid), a substance for suppressing translation of protein from mRNA of SMS2 (such as nucleic acid), a substance for degrading protein of translated protein of SMS2, a substance for destabilizing translated protein of SMS2, a substance for decreasing enzyme activity of protein of SMS2 (such as an antibody having neutralization activity), a variant of N-SMase2 in which enzyme activity is reduced or lost, a nucleic acid encoding it or an expression vector comprising the nucleic acid, or the like (or a substance for replacing it with a natural type by recombining (e.g., recombinant vector)), or the like. Nucleic acids (siRNA, antisense oligonucleotide, ribozyme or expression vectors thereof, and the like), protein, peptide, or other low molecules (e.g., those that can be synthesized by combinatorial chemistry or the like), high molecules, and a complex of these substances and the like are also encompassed. As for the subject substance, exemplified are, without limitation, siRNA, antisense oligonucleotide, ribozyme or expression vectors thereof, and other nucleic acids.

### DESCRIPTION OF PREFERRED EMBODIMENTS

It should be understood that while description of preferred embodiments is described below, the embodiments are illustrative of the present invention and the scope of the present invention is not limited to such preferred embodiments. It should be also understood that those skilled in the art can readily carry out modification, alternation or the like within the scope of the present invention with reference to the preferred embodiments below.

### (Method for Searching for Medicament)

In one aspect, the present invention provides a method for screening a treatment substance or prevention substance for a disease associated with amyloid β, The method comprises the steps of: (1) allowing protein of neutral sphingomyelinase 2 (N-SMase2) and/or sphingomyelin synthetic enzyme 2 (SMS2) to contact with a test substance; (2) comparing enzyme activity of the protein of the N-SMase2 and/or SMS2 to which the test substance has been contacted, with enzyme activity of protein of the N-SMase2 and/or SMS2 to which the test substance has not been contacted; and (3) when the enzyme activity of the protein of the N-SMase2 to which the test substance has been contacted is increased compared to the enzyme activity of the protein of the N-SMase2 to which the test substance has not been contacted, and/or the enzyme activity of the protein of the SMS2 to which the test substance has been contacted is decreased compared to the enzyme activity of the protein of the SMS2 to which the test substance has not been contacted, selecting the test substance as a treatment substance or prevention substance of the disease associated with amyloid β.

In another aspect, a method for screening a treatment substance or prevention substance for a disease associated with amyloid β according to the present invention comprises: (1) allowing a cell to contact with a test substance; (2) comparing expression of N-SMase2 and/or SMS2 in the cell to which the test substance has been contacted, with expression of N-SMase2 and/or SMS2 in a control cell to which the test substance has not been contacted; and (3) when the expression of the N-SMase2 in the cell to which the test substance has been contacted is increased compared to the expression of the N-SMase2 in the control cell to which the test substance has not been contacted, and/or when the expression of SMS2 in the cell to which the test substance has been contacted is decreased compared to the expression of SMS2 in the cell to which the test substance has not been contacted, selecting the test substance as a treatment substance or prevention substance of the disease associated with amyloid β. In a preferred embodiment, the cell and the control cell to be used are a nerve cell, and more preferably a nerve cell obtained from the brain.

In another aspect, a method for screening a treatment substance or prevention substance for a disease associated with amyloid β according to the present invention comprises: (1) allowing a cell to contact with a test substance; (2) comparing an exosome secretion level in the cell to which the test substance has been contacted, with an exosome secretion level in a control cell to which the test substance has not been contacted; and (3) when an exosome secretion level in the cell to which the test substance has been contacted is increased compared to an exosome secretion level in the control cell to which the test substance has not been contacted, selecting the test substance as a treatment substance or prevention substance of the disease associated with amyloid β. In a preferred embodiment, the cell and the control cell to be used are a nerve cell, and more preferably a nerve cell obtained from the brain.

In yet another aspect, the present invention provides a screening method for a medicament for the treatment or prevention of a condition, disorder or disease associated with amyloid β. This method comprises the steps of: A) subjecting at least one of elements selected from the group consisting of 1) exosome; 2) neutral sphingomyelinase 2 (N-SMase2); and 3) sphingomyelin synthetic enzyme 2 (SMS2) and a candidate of the medicament in a condition in which they can interact with each other; and B) examining an influence by the candidate of the medicament to the element, wherein at least one of the elements is an index for determining as to whether or not the candidate is the medicament. The association had not been known between an exosome and aggregation or fibrosis of amyloid β, or the clearance thereof, and the present invention provides a novel method for searching for a medicament for treating or preventing a disease associated with amyloid β. In this regard, the present invention is worthy of being focused in the subject field. In addition, the relationship had not been known between aggregation or fibrosis of exosome and amyloid β, and sphingomyelin metabolism (and in particular, SMS2 and nSMase2), and the present invention provides a novel method for searching for a medicament for treating or preventing a disease associated with amyloid β. In this regard, the present invention is worthy of being focused in the subject field.

The screening method according to the present invention may be performed with a reconstituted system in vitro or with a cell. In one embodiment, the screening method according to the present invention allows an exosome to contact with Aβ1-40 or Aβ1-42 in the presence or absence of a candidate, and based on the result, the present invention is even capable of screening a regulatory factor for the polymerization or fibrosis of the Aβ1-40 or Aβ1-42. The screening method according to the present invention also allows Aβ1-40 or Aβ1-42 to contact with microglia in the presence or absence of a candidate, and in the presence of an exosome, and based on the result, is capable of screening a regulatory factor for the intake of the Aβ1-40 or Aβ1-42 into the microglia. The present invention also examines the activity of N-SMase2 and/or SMS2 in the presence or absence of a candidate, and based on the result, the present invention is capable of screening a regulatory factor for the secretion of the exosome Herein, the decrease in the activity of N-SMase2 and the increase in the activity of SMS2 are an index for the increase in secretion of the exosome. The present invention also examines the activity of N-SMase2 and/or SMS2 in the presence or absence of a candidate, and based on the result, the present invention is capable of screening a regulatory factor for the polymerization or fibrosis of Aβ1-40 or Aβ1-42. Herein, the decrease in the activity of N-SMase2 and the increase in the activity of SMS2 are an index for the decrease in polymerization or fibrosis of Aβ1-40 or Aβ1-42.

In the method according to the present invention, it is understood that assay of the amount or level of exosome, the amount or level of Aβ1-40, the amount or level of Aβ1-42, the amount or level of SMS2 and the amount or level of N-SMase2 can be performed immunologically or physicochemically (including mass spectrometry and the like), and the assay for SMS2 or N-SMase2 can be performed by assaying enzyme activity. Such assay of enzyme activity can be actualized by any method that is publicly known in the subject field or that is described herein.

For example, for the assay of Aβ amount, a specific antibody or the like can be used, and Aβ1-X ELISA by IBL Co., Ltd or the like can be utilized in immune response, such as ELISA. For example, in an immunostaining method, an anti-Aβ antibody 4G8 (Covance) which binds to an Aβ fragment is mixed with protein G sepharose, and the mixture is immunoprecipitated under appropriate conditions (e.g., overnight at 4° C.), followed by washing under appropriate conditions (e.g., washing 5 times with a wash buffer solution (wash buffer) (50 mM Tris-HCl, pH7.6, 150 mM NaCl, 2 mM EDTA, Complete inhibitor by Roche), and washing 3 times with ultrapure water); and the purified matter can be detected or quantified by an immunostaining method using 82E1 (IBL), which is an Aβ N-Terminal truncated antibody.

Alternatively, in an exemplified embodiment, the assay of the total amount, level or the like of Aβ1-40, Aβ1-42 or an Aβ fragment including the Aβ1-40 and Aβ1-42 in the present invention can be performed by a γ-secretase assay system. For example, in such an assay system, the assay can be performed using an appropriate buffer (e.g., Buffer C (300 mM citric acid, pH6.0, 500 mM sucrose, 0.5% CHAPSO, 0.2% phosphatidylcholine, 20 mM bestatin (Bestatin), 20 mM amastatin (Amastatin), 10 mM phenanthroline (Phenanthroline), 20 mM captopril (Captopril), ×10 Complete inhibitor by Roche) available from Roche), and by using an enzyme which is available through a publicly known method, γ-secretase complex (e.g., the one obtained by purifying a membrane protein fraction, which is extracted using a surfactant (e.g., CHAPSO) from a partially purified membrane fraction of a cultured cell, by an immunoprecipitation method using an anti-nicastrin antibody, from Kakuda et al., J. Biol. Chem. 2006 May 26; 281(21): 14776-86. Epub 2006 Apr. 4., or the like). The reaction can be performed by adding an appropriate substrate peptide (e.g., Aβ42 (consisting of amino acids 1 to 42 of SEQ ID NO: 89)) to an enzyme (e.g., purified γ-secretase enzyme) which is diluted with an appropriate buffer (e.g., above-mentioned Buffer C), followed by incubation for an appropriate time and at an appropriate temperature (e.g., 37° C., 1 hour). In addition, an experiment can be performed to examine with regard to γ-secretase specific cleavage, in which a γ-secretase inhibitor (e.g., DAPT (available from Peptide Institute, Inc)) is added, as a control, at an appropriate final concentration (e.g., 10 μM), thus performing a control experiment. After a reaction has occurred, the reaction may be stopped using an appropriate method (e.g., by placement on ice), and the sample supernatant may be subjected to an appropriate analysis method (e.g., MALDI TOF MS analysis), thus performing the analysis. During the analysis, the sample may be purified as appropriate to increase the purity thereof. For example, part of the sample (enzyme reaction liquid) can be purified by mixing an appropriate specific binding substance (e.g., anti-Aβ antibody 4G8 (Covance)), which binds to a cleaved peptide or substrate peptide, or to both of them, and protein G sepharose, followed by immunoprecipitating under appropriate conditions (e.g., overnight at 4° C.), and washing under appropriate conditions (washing 5 times with a wash buffer solution (wash buffer) (50 mM Tris-HCl, pH7.6, 150 mM NaCl, 2 mM EDTA, Complete inhibitor by Roche) and washing 3 times with ultrapure water). The immunoprecipitate can be eluted under appropriate conditions (e.g., trifluoroacetic acid/acetonitrile/water (1:20:20)), and then mass spectrometry can be performed using MALDI-TOF MS or the like.

Alternatively, respective elements can be identified and/or detected or quantified by using antibodies specific to respective elements and through immune reaction (e.g., ELISA).

In the screening method according to the present invention, in addition to a candidate substance, it is also possible to include a substance known to have pharmacological activity as a positive control, and/or a substance known not to have a pharmacological activity as a negative control. Furthermore, the candidate substance to be the object, may be any substance.

In one embodiment, in the screening method of the present invention, the step A) is a step of subjecting a cell and a candidate in a condition in which they can interact with each other, and step B) is a step of examining a secretion level of an exosome, wherein the secretion level of the exosome from the cell is used as an index for determining as to whether or not the candidate is the medicament. Accordingly, in one embodiment, the method according to the present invention may comprise a step of determining as to whether or not the candidate is the medicament based on a result of examining the secretion level of the exosome from the cell. Herein, when the secretion level of the exosome is increased, it is considered that the capability for clearance by the amyloid β is also increased. Thus, the candidate that causes the increase can be determined as having a capability as a medicament for treating or preventing a disease associated with amyloid β. In a preferred embodiment, the cell to be used and the object cell are a nerve cell, and more preferably a nerve cell obtained from the brain.

In one embodiment, in the present invention, the element includes an exosome, and the present invention includes a step of allowing the exosome to contact with Aβ1-40 and/or Aβ1-42 in the presence or absence of a candidate of medicament, wherein the amount of at least one of the exosome, Aβ1-42 and Aβ polymer is used as an index for determining as to whether or not the candidate is the medicament. Herein, when the amount of exosome is increased, it is considered that the capability for clearance by the amyloid β is also increased. Thus, the candidate that causes the increase can be determined as having a capability as a medicament for treating or preventing a disease associated with amyloid β. On the other hand, when the amount of the Aβ1-40, Aβ1-42 or Aβ polymer is decreased, the candidate that causes the decrease can be determined as having a capability as a medicament for treating or preventing a disease associated with amyloid β.

In one embodiment, in the present invention, the element includes an exosome, and the present invention includes a step of allowing the exosome to contact with Aβ1-40 and/or Aβ1-42 and microglia in the presence or absence of a candidate of medicament, wherein the intake of the exosome and/or Aβ1-40 and/or Aβ1-42 into the microglia is used as an index for determining as to whether or not the candidate is the medicament. Herein, when a free exosome is decreased, or the intake of the exosome into microglia is increased, it is considered as a result of the capability for clearance by amyloid β has been increased. Thus, the candidate that causes the phenomenon can be determined as having a capability as a medicament for treating or preventing a disease associated with amyloid β. On the other hand, when the Aβ1-40 and/or Aβ1-42 is decreased, or the intake of the Aβ1-40 and/or Aβ1-42 into microglia is increased, the candidate that causes the phenomenon can be determined as having a capability as a medicament for treating or preventing a disease associated with amyloid β.

In one embodiment, the present invention comprises a step of examining activity of N-SMase2 and/or SMS2 in the presence or absence of a candidate of medicament, and the decrease in the activity of the NSMase2 and the increase in the activity of the SMS2 are used as an index for determining as to whether or not the candidate is the medicament.

In one embodiment, said condition, disorder or disease is one or more selected from the group consisting of Alzheimer's disease, retinal disease (e.g., age-related macular degeneration (also referred to as age-related macular retinopathy), glaucoma and the like) (see Journal of Pharmacological Sciences, Vol. 134 (2009), No. 6, 309-314 and the like).

(Medicament, Prevention and Treatment of a Condition, Disorder or Disease Associated with Amyloid β)

In one aspect, the present invention provides a pharmaceutical composition for treating or preventing a disease associated with amyloid β, comprising a substance for increasing enzyme activity or expression of protein of N-SMase2. In this aspect, the present invention may be provided as a substance for increasing enzyme activity or expression of protein of N-SMase2, for treating or preventing a disease associated with amyloid β; or as a method for treating or preventing a disease associated with amyloid β in a subject, the method comprising: administering an effective amount of the substance for increasing enzyme activity or expression of protein of N-SMase2 to a subject in need of such a treatment or prevention.

In one embodiment, the substance for increasing enzyme activity or expression of the protein of N-SMase2 in the present invention may be a protein and/or expression vector of N-SMase2 (nSMase2) or a functionally equivalent variant thereof (e.g., derivative, partial peptide and the like). Alternatively, the factor of such N-SMase2 (nSMase2) may be a gene treatment system to which N-SMase2 (nSMase2) is introduced. The gene treatment system may be a naked gene; or it may be introduced into a living organism by either of a conventional virus or non-virus vector, or retro virus vector or liposome subsumption form or the like. The gene of N-SMase2 (nSMase2) may be genomic DNA, cDNA, mDNA or synthetic DNA.

The N-SMase2 typically includes SEQ ID NO: 84 (human) (NM_{—}018667, 5269 bp), SEQ ID NO: 86 (mouse) (NM_{—}021491, 5148 bp), rat (NM_{—}053605, 5022 bp), (full length sequence of N-SMase2) and the like, but it is understood that, besides the ones described above, any sequences can be used as a target as long as such sequences are known as N-SMase2. Such a sequence includes those that are referred to by a plurality of Accession numbers on a genomic database.

Besides the above-mentioned proteins, such proteins which have, for example, high homology with the sequences described in these Accession numbers (normally 70% or more, preferably 80% or more, more preferably 90% or more, and most preferably 95% or more) and which have functions that the above-mentioned proteins have (such as a function of synthesizing sphingomyelin in a cell, or the like) are included in the protein according to the present invention. It is also understood that a protein is also comprised which consists of an amino acid sequence, in which one or more amino acids are added, deleted, substituted or inserted, in the amino acid sequences described in the Accession numbers associated with the above-mentioned proteins, wherein the number of amino acids that usually vary is within 30 amino acids, preferably within 10 amino acids, more preferably 5 amino acids, and most preferably 3 amino acids. Alternatively, a protein is also comprised which has high homology with a DNA sequence described in the Accession number associated with the above-mentioned nucleotide sequence. High homology means homology of 50% or more, preferably 70% or more, even more preferably 80% or more, and more preferably 90% or more (such as 95% or more, and further 96%, 97%, 98% or 99% or more). This homology can be determined by the mBLAST algorithm (Altschul et al. (1990) Proc. Natl. Acad. Sci. USA 87: 2264-8; Karlin and Altschul (1993) Proc. Natl. Acad. Sci. USA 90: 5873-7). Alternatively, the sequence targeted by the present invention may be a sequence which hybridizes under stringent conditions with a DNA sequence described in the Accession number associated with the above-mentioned nucleotide sequence. Herein, “stringent conditions” include such conditions as, for example, “2×SSC, 0.1% SDS, 50° C.”, “2×SSC, 0.1% SDS, 42° C.”, “1×SSC, 0.1% SDS, 37° C.”, and more stringent conditions include “2×SSC, 0.1% SDS, 65° C.”, “0.5×SSC, 0.1% SDS, 42° C.” and “0.2×SSC, 0.1% SDS, 65° C.”.

From the above-mentioned proteins with high homology, those skilled in the art can appropriately obtain a protein which is functionally equivalent to the above-mentioned proteins, by using a method for assaying degradation activity of sphingomyelin. A specific method for assaying activity will be exemplarily described in the Examples below. Furthermore, it is possible for those skilled in the art to appropriately obtain an endogenous gene, corresponding to the above-mentioned gene, in another living organism, based on the base sequence of the above-mentioned gene. It should be noted that as used herein, the above-mentioned protein and gene corresponding to the above-mentioned protein and gene in living organisms other than humans, or the above-mentioned protein and gene which are functionally equivalent to the above-stated protein and gene, may also be simply described with the above-mentioned names.

The method for increasing the expression of a specific endogenous gene such as N-SMase2 may also be a method for exogeneouslly introducing an expression vector or the like, such as a gene therapy method.

In a certain embodiment according to the present invention, “expression vector” is preferably, independently replicable in a host cell, and at the same time, is preferably constituted of a promoter, a ribosome-binding sequence, a nucleic acid of N-SMase2 set forth by SEQ ID NO: 83, 85 and the like or a functionally-equivalent variant thereof, and a transcription termination sequence. Furthermore, a gene for controlling a promoter may be comprised. It is understood that the above-mentioned vector may be prepared in accordance with a publicly known method and any form described herein or other publicly known forms can be applied.

In a certain embodiment, a therapeutically effective amount of N-SMase2 is administered by a method known to those skilled in the art as a “gene treatment”. The gene treatment used herein refers to a general method for treating a pathological condition of a subject by inserting an exogenous nucleic acid to an appropriate cell of the subject. The nucleic acid is inserted into a cell in such a manner to maintain the function thereof, e.g., in such a manner to maintain the ability to express a specific polypeptide. In some embodiments, a therapeutically effective amount of N-SMase2 is administered through viral gene therapy, and using a virus vector transfer cassette (such as a cassette of retrovirus, adenovirus or adeno-associated virus) comprising a nucleic acid sequence encoding N-SMase2.

The preferable subject in the present invention is a vertebrate animal subject. A preferable vertebrate animal is a warm-blooded animal. A preferable warm-blooded, vertebrate animal is a mammal. While the subject treated by the currently disclosed method is preferably a human, it is understood that the principle of the present invention indicates the effectiveness to all the vertebrate animal species included in the term “subject”. In this configuration, it is understood that the vertebrate animal is all the vertebrate animal species that are desired to be treated for disorders. The “subject” as used herein includes both human and animal subjects. Thus, treatment applications for animals are provided in accordance with the present invention.

In another aspect, provided is a pharmaceutical composition for treating or preventing a disease associated with amyloid β, comprising a substance for suppressing enzyme activity or expression of protein of SMS2. In this aspect, the present invention may be provided as a substance for suppressing enzyme activity or expression of the protein of SMS2 for treating or preventing a disease associated with amyloid β. Alternatively, in this aspect, the present invention may be provided as a method for treating or preventing a disease associated with amyloid β in a subject, the method comprising: administering an effective amount of a substance for suppressing enzyme activity or expression of the protein of SMS2 to the subject in need of such a treatment or prevention.

In one embodiment, the substance for suppressing enzyme activity or expression of protein of SMS2 that is used in the present invention may be a nucleic acid for suppressing the expression of SMS2. As for the nucleic acid for suppressing the expression of SMS2 as used herein, it is understood that any nucleic acid may be used that is described in (Nucleic Acid for Suppressing the Expression of SMS2) in the present specification.

Alternatively, the present invention provides a nucleic acid (e.g., siRNA, and antisense nucleic acid) for suppressing expression of SMS2 for treating or preventing a condition, disorder or disease associated with amyloid β. As for the nucleic acid for suppressing expression of SMS2 that is used for treating or preventing a condition, disorder or disease associated with amyloid β, it is understood that any nucleic acid described in (Nucleic Acid for Suppressing the Expression of SMS2) in the present application can be used.

In one embodiment, such a nucleic acid is a siRNA and/or antisense nucleic acid. It is possible to exemplify a specific siRNA or antisense nucleic acid described in (Nucleic Acid for Suppressing the Expression of SMS2).

In another embodiment, such an siRNA consists of any one or more selected from the group consisting of the siRNAs described in the following (a) to (p):

(a) an siRNA wherein one strand of the duplex RNA portion is a base sequence set forth by SEQ ID NO: 1 and the other strand of the duplex RNA portion is a base sequence set forth by SEQ ID NO: 2;
(b) an siRNA wherein one strand of the duplex RNA portion is a base sequence set forth by SEQ ID NO: 3 and the other strand of the duplex RNA portion is a base sequence set forth by SEQ ID NO: 4;
(c) an siRNA wherein one strand of the duplex RNA portion is a base sequence set forth by SEQ ID NO: 5 and the other strand of the duplex RNA portion is a base sequence set forth by SEQ ID NO: 6;
(d) an siRNA wherein one strand of the duplex RNA portion is a base sequence set forth by SEQ ID NO: 7 and the other strand of the duplex RNA portion is a base sequence set forth by SEQ ID NO: 8;
(e) an siRNA wherein one strand of the duplex RNA portion is a base sequence set forth by SEQ ID NO: 9 and the other strand of the duplex RNA portion is a base sequence set forth by SEQ ID NO: 10;
(f) an siRNA wherein one strand of the duplex RNA portion is a base sequence set forth by SEQ ID NO: 11 and the other strand of the duplex RNA portion is a base sequence set forth by SEQ ID NO: 12;
(g) an siRNA wherein one strand of the duplex RNA portion is a base sequence set forth by SEQ ID NO: 13 and the other strand of the duplex RNA portion is a base sequence set forth by SEQ ID NO: 14;
(h) an siRNA wherein one strand of the duplex RNA portion is a base sequence set forth by SEQ ID NO: 15 and the other strand of the duplex RNA portion is a base sequence set forth by SEQ ID NO: 16;
(i) an siRNA wherein one strand of the duplex RNA portion is a base sequence set forth by SEQ ID NO: 17 and the other strand of the duplex RNA portion is a base sequence set forth by SEQ ID NO: 18;
(j) an siRNA wherein one strand of the duplex RNA portion is a base sequence set forth by SEQ ID NO: 19 and the other strand of the duplex RNA portion is a base sequence set forth by SEQ ID NO: 20;
(k) an siRNA wherein one strand of the duplex RNA portion is a base sequence set forth by SEQ ID NO: 21 and the other strand of the duplex RNA portion is a base sequence set forth by SEQ ID NO: 22;
(l) an siRNA wherein one strand of the duplex RNA portion is a base sequence set forth by SEQ ID NO: 23 and the other strand of the duplex RNA portion is a base sequence set forth by SEQ ID NO: 24, which is a complementary sequence thereof;
(m) an siRNA wherein one strand of the duplex RNA portion is a base sequence set forth by SEQ ID NO: 25 and the other strand of the duplex RNA portion is a base sequence set forth by SEQ ID NO: 26, which is a complementary sequence thereof;
(n) an siRNA wherein one strand of the duplex RNA portion is a base sequence set forth by SEQ ID NO: 27 and the other strand of the duplex RNA portion is a base sequence set forth by SEQ ID NO: 28, which is a complementary sequence thereof;
(o) an siRNA wherein one strand of the duplex RNA portion is a base sequence set forth by SEQ ID NO: 43 and the other strand of the duplex RNA portion is a base sequence set forth by SEQ ID NO: 44;

(p) an siRNA according to any of (a) to (O), wherein one to several nucleotides are added, inserted, deleted or substituted in one or both of the base sequences, and having an activity of suppressing the expression of SMS2.

In one embodiment, the medicament or pharmaceutical composition according to the present invention further comprises a pharmaceutically acceptable excipient. When the composition of the present invention is used as a medicament or pharmaceuticals, the dosage form includes, for example, tablets, powders, fine granules, granules, coated tablets, controlled release formulations, capsules, injections or the like. The pharmaceuticals can comprise an excipient, optionally, additives such as a binding agent, a disintegrating agent, a lubricant, a flavoring agent, a coloring agent, a delayed releasing agent or the like. In a case of an oral formulation, as an excipient, for example, lactose, corn starch, sucrose, glucose, mannitol, sorbite, crystalline cellulose or the like; as a binding agent, for example, polyvinyl alcohol, polyvinylether, methylcellulose, hydroxylpropylcellulose, gum arabic, tragacanth, gelatin, shellac, polyvinylpyrrolidone, block copolymers or the like; as a disintegrating agent, for example, starch, agar, gelatin powder, crystalline cellulose, calcium carbonate, sodium bicarbonate, calcium citrate, dextrin, pectin or the like; as a lubricant, for example, magnesium stearate, talc, polyehylene glycol, silica, hydrogenated vegetable oils or the like; as a flavoring agent, for example, cocoa powder, peppermint oil, cinnamon powder or the like can be used, but not limited thereto. Optionally, it can be coated so as to be a controlled release formulation or enteric coated formulation. In a case of a formulation for injection, a pH modulating agent, a solubilizing agent, an isotonic agent, a buffering agent or the like can be used, but not limited thereto.

The medicament or pharmaceutical composition of the present invention can be mixed with a physiologically acceptable carrier, excipient or diluent or the like as above, and orally or parenterally administered as a pharmaceutical composition. As an oral formulation, it can be in dosage forms above such as granules, powders, tablets, capsules, solvents, emulsions, or suspensions. As a parenteral formulation, dosage forms such as injections, drops, medicines for external use, inhalant (nebulizer) or suppository or the like can be selected. As injections, hypodermic injections, intramuscular injections, intraperitoneal injections, intracranial injections, or intranasal injections or the like can be shown. As a medicine for external use, transnasal drugs or ointments or the like can be shown. Technique for formulating the above dosage form so as to comprise the medicament of the present invention as a major ingredient is known.

For example, tablets for oral administration can be made by adding to the nucleic acid or medicament of the present invention an excipient, a disintegrating agent, a binding agent, a lubricant or the like, mixing them and compression molding them. As an excipient, lactose, starch or mannitol or the like are generally used. As a disintegrating agent, calcium carbonate or carboxymethyl cellulose calcium or the like are generally used. As a binding agent, gum arabic, carboxymethyl cellulose or polyvinylpyrrolidone is used. As a lubricant, talc or magnesium stearate or the like are known.

When the medicament or pharmaceutical composition according to the present invention is a tablet, the tablet can be made with known coating so as to make them to be masked or an enteric coated formulation. As a coating agent, ethylcellulose or polyoxyethylene glycol or the like can be used.

Injections can also be obtained by dissolving the nucleic acid or medicament of the present invention as a major ingredient with a suitable dispersing agent, or dissolving or dispersing them in a disperse medium. Depending on selecting the disperse medium, any dosage form of an aqueous solvent or an oily solvent can be taken. For making an aqueous solvent, a distilled water, a saline, or Ringer's solution or the like are used as a disperse medium. For an oily solvent, various vegetable oils or propylene glycol or the like is used as a disperse medium. In such a case, preservative agents such as paraben or the like can be optionally added. Into injections, known isotonic agents such as sodium chloride or glucose or the like can also be added. Furthermore, analgesics such as benzalkonium chloride or procaine hydrochloride can be added.

The medicament or pharmaceutical composition of the present invention can also be made to be a medicament for external use by making it to be a solid, liquid or semi-solid composition. A solid or liquid composition can be made as a medicament for external use by making them to be similar to the composition as described above. A semi-solid composition can be prepared by optionally adding a thickening agent to a suitable solvent. For the solvent, water, ethyl alcohol or polyethylene glycol or the like can be used. For a thickening agent, bentonite, polyvinyl alcohol, acrylic acid, methacrylic acid, or polyvinylpyrrolidone or the like are generally used. To the composition, a preservative agent such as benzalknoum hydrochloride or the like can be added. It can also be made to be a suppository by combining with, as a carrier, an oily substrate such as cacao butter or an aqueous gel substrate such as cellulose derivatives.

When the medicament or pharmaceutical composition of the present invention is used as a gene therapeutic agent, a method of directly administering the nucleic acid or medicament of the present invention by an injection, as well as a method of administering a vector wherein the nucleic acid is incorporated are listed. As the vector, an adenoviral vector, an adeno-associated viral vector, a herpesviral vector, a vaccinia viral vector, a retroviral vector, a lentiviral vector or the like are listed, and it can be effectively administered by using these viral vectors.

It is also possible to introduce the medicament or pharmaceutical composition of the present invention into a phospholipid vesicle such as a liposome and administer it. A vesicle retaining an siRNA or shRNA can be introduced into a given cell by lipofection method. Then, the cell thus obtained is administered systemically, for example, intravenously, intraarterially or the like. It can also be administered locally to site or the like of obesity, diabetes, dyslipidemia and fatty liver. Since siRNA exerts a highly excellent effect of specific and post-transcriptional suppression in vitro but is quickly degraded by a nuclease activity in a serum and has limited duration time in vivo, a more optimal and effective delivery system has been sought to be developed. As one example, by Ochiya, T et al; Nature Med., 5: 707-710, 1999, Curr. Gene Ther., 1:31-52, 2001, it was reported that when atelocollagen that is a biocompatible material is mixed with a nucleic acid to form a complex, it has an effect of protecting the nucleic acid from degrading enzymes in vivo and thus is a carrier which is very suitable as a carrier for an siRNA. Therefore, such a form can be used, but a method for introducing the nucleic acid or medicament of the present invention is not limited thereto. Since it is not degraded in vivo quickly by nucleic acid degrading enzymes in a serum, a long-lasting effect can be achieved in such a way. For example, in Takeshita F. PNAS. (2003) 102(34) 12177-82, Minakuchi Y Nucleic Acids Research (2004) 32(13) e109, it was reported that atelocollagen derived from a bovine skin forms a complex with a nucleic acid and has an effect of protecting the nucleic acid from degrading enzymes in vivo, which is thus very suitable as a carrier for an siRNA. Such techniques can be used.

The medicament or pharmaceutical composition of the present invention is administered to a mammal including a human in a necessary amount (an effective amount) which is within the scope of dosage amount that is deemed as safe. The dosage amount for the nucleic acid or medicament of the present invention can be suitably determined, ultimately by the judgment of a doctor or vetenarian in view of the kind of dosage form, administration method, age or body weight of a patient, symptom of a patient or the like. As an example, although it varies depending on age, sex, symptom, route for administration, times of administration, dosage form, for example, the dosage amount in a case of an adenovirus is about 10⁶-10¹³ per one administration per day and is administered at interval of 1 to 8 weeks.

It is also possible to use a commercially available kit for transgenesis (for example, Adeno Express: Clontech) in order to introduce a siRNA or shRNA into a tissue or organ of interest.

The medicament or pharmaceutical composition of the present invention can further comprise an effective ingredient. Such additional medicaments that can be comprised are variously considered depending on its purpose.

In another aspect, the present invention provides use of a nucleic acid (for example, siRNA, and antisense nucleic acid) for suppressing the expression of SMS2 of the present invention, for the manufacture of a medicament for treating or preventing a condition, disorder or disease associated with amyloid β. Here, it is understood that as a nucleic acid that suppresses the expression of SMS2 of the present invention, any nucleic acid described in section (Nucleic Acid for Suppressing the Expression of SMS2) or the present section can be used.

In another aspect, the method of the present invention is a method for manufacturing a pharmaceutical composition or medicament for treating or preventing a condition, disorder or disease associated with amyloid β, comprising a nucleic acid for suppressing the expression of SMS2, the method comprising: mixing the nucleic acid that suppresses the expression of SMS2 with a pharmaceutically acceptable excipient. Besides a medicament form, food, health food, functional food or the like which are approved by the authorities can be similarly manufactured. In such a case, in place of a pharmaceutically excipient, a secondary ingredient can be used depending on its purpose.

An effective amount of the medicament or pharmaceutical composition of the present invention refers to an amount in which the medicament or pharmaceutical composition of the present invention can exert an intended pharmaceutical efficacy, and the smallest concentration among such effective amounts is sometimes referred herein as a minimal effective amount, which can be suitably determined by those skilled in the art based on the description of the present specification. For determining such effective amounts, besides actual administration, animal model or the like can be used. The present invention is also useful in determining such effective amounts.

In a further aspect, the present invention provides a method for treating or preventing a condition, disorder or disease associated with amyloid β, the method comprising: administering the nucleic acid (for example, siRNA, antisense nucleic acid) that suppresses the expression of SMS2 of the present invention to a subject in need of the treatment or prevention. Here, it is understood that as a nucleic acid that suppresses the expression of SMS2 which can be used, any nucleic acid described in the previous section (Nucleic Acid for Suppressing the Expression of SMS2) or the present section can be used.

As to the administration to a subject or to an individual, any method can be used as described in the present section, and the administration can be generally carried out by a method publicly known to those skilled in the art, such as intraarterial injection, intravenous injection, hypodermic injection or the like. Dosage amounts vary depending on body weight or age of a patient, administration method or the like, but those skilled in the art (doctors, veterinarians, pharmacists or the like) can suitably select a reasonable dosage amount.

An individual that is an object for the method of treatment or prevention of the present invention is not specifically limited as long as it is an organism that can develop a condition, disorder or disease associated with amyloid β, but preferably a human.

The amount of an effective ingredient used in the medicament of composition of the present application, or an effective ingredient used in the method for treatment or prevention of the present invention, can be readily determined by those skilled in the art by taking into consideration purpose of use, objective diseases (kind, severity or the like), age, body weight, sex, anamnesis of a patient, form or kind of cell, or the like. Frequency at which the method of treatment of the present invention is carried out on a subject (or a patient) can also be readily determined by those skilled in the art in view of purpose of use, objective diseases (kind, severity or the like), age, body weight, sex, anamnesis of a patient, course of treatment or the like. Such frequency includes, for example, administration once per day to once per several months (for example, once per week to once per month). It is preferable to carry out administration once per day to once per month with observing the course.

The type and amount of ingredients used in the method of treatment of the present invention can be readily determined by those skilled in the art in view of purpose of use, objective diseases (kind, severity or the like), age, bodyweight, sex, anamnesis of a patient, form or type of site of administration of a patient or the like, based on the information obtained from the method of the present invention (for example, information regarding a disease). Frequency at which the method of monitoring of the present invention is administered on a subject (or a patient) can also be readily determined by those skilled in the art in view of purpose of use, objective diseases (kind, severity or the like), age, body weight, sex, anamnesis of a patient, course of treatment or the like. The frequency of monitoring a status of a disease includes, for example, monitoring once per day to once per several months (for example, once per week to once per month). It is preferable to carry out monitoring once per day to once per month while observing the course.

The present invention can be used as a kit or the like, in such a case, it can be accompanied with written instructions. “Written instructions” herein refers to ones wherein the method of treatment of the present invention is described to a human who carries out the administration such as a doctor, a patient or the like. The written instructions describe, for example, a statement that instructs to suitably administer the medicament or the like of the present invention. The written instructions are made according to the form which is defined by the authorities of a country in which the present invention is carried out (for example, Ministry of Health, Labour and Welfare in Japan, Food and Drug Administration in the United State of America (FDA) or the like), and explicitly describes that it is approved by the authorities. The written instructions are so-called appendant document (package insert), which is usually provided in paper media but not limited thereto, and can be provided, for example, in forms such as electronic media (for example, website provided by the Internet, and electronic mail).

### (Nucleic Acid for Suppressing the Expression of SMS2)

The present invention provides a nucleic acid which suppresses the expression of SMS2, and in particular, the novel use thereof. The nucleic acid of the present invention has a function that suppresses the translation or transcription or the like of a nucleic acid. Such a nucleic acid can include an antisense nucleic acid, a nucleic acid having RNAi effect (for example, siRNA), a nucleic acid having a ribozyme activity, or the like. The nucleic acid which suppresses the expression of SMS2 of the present invention can comprise a modified nucleic acid.

Such a nucleic acid (e.g., siRNA, and antisense nucleic acid) is used for ameliorating, treating or preventing a condition, a neural disease and the like (e.g., Alzheimer's disease and the like) associated with aggregation of amyloid β (Aβ).

A preferred embodiment of the nucleic acid which suppresses the expression of SMS2 according to the present invention, such as siRNA of SMS2, can include, for example, nucleic acids selected from the group consisting of the following (a) to (c): (a) an antisense nucleic acid toward a transcript of a gene encoding an SMS2 protein or part thereof, (b) a nucleic acid having a ribozyme activity which specifically cleaves a transcript encoding an SMS2 protein; and (c) a nucleic acid having an effect of inhibiting the expression of a gene encoding SMS2 protein via RNAi effect (for example, siRNA).

SMS2 can include, typically, SEQ ID NO: 79 (human) (Locus: NM_{—}152621, 6246 bp), SEQ ID NO: BO (mouse) (NM_{—}028943, 5791 bp) (full length sequence of SMS2) or the like. However, it is understood that, besides the ones above, any sequence known as SMS2 can be used as a target. As such sequences, sequences referred to by a plurality of Accession numbers on genome databases (for example, on nucleotide databases, besides above, NM_{—}001136257, NM_{—}001136258, BC041369, BC₀₂₈₇₀₅ (human) or the like are searched, and on protein databases, NP_{—}001129730, NP_{—}689834, NP_{—}001129729, Q8NHU3, AAH41369, AAH28705, Q86VZ5 (the preceding are human), NP_{—}083219 (mouse) or the like) are searched on the public gene database NCBI.

Even if it is not the above proteins, for example, proteins which have a high homology (usually 70% or more, preferably 80% or more, more preferably 90% or more, most preferably 95% or more) to the sequences described in these Accession numbers and have functions which the above proteins have (for example, a function of synthesizing sphingomyelin in a cell, or the like), are encompassed in proteins which the present invention targets. It is understood that proteins consisting of an amino acid sequence wherein one or more of amino acids are added, deleted, substituted, or inserted in the amino acid sequences set forth by the Accession numbers related to the above proteins and wherein the number of amino acids that are changed is usually within 30 amino acids, preferably within 10 amino acids, more preferably within 5 amino acids and most preferably within 3 amino acids are also encompassed. Alternatively, ones having a high homology to the DNA sequences set forth by the Accession numbers related to the above nucleotide sequences are also encompassed. High homology means 50% or more, preferably 70% or more, more preferably 80% or more, furthermore preferably 90% or more (for example, 95% or more, further 96%, 97%, 98% or 99% or more) of homology. Such homology can be determined by mBLAST algorithm (Altschul et al. (1990) Proc. Natl. Acad. Sci. USA 87: 2264-8; Karlin and Altschul (1993) Proc. Natl. Acad. Sci. USA 90: 5873-7). Alternatively, the sequence which the present invention targets can be one which hybridizes under stringent condition to the DNA sequences set forth by Accession numbers related to the above nucleotide sequences. Here, “stringent condition” can include, for example, “2×SSC, 0.1% SDS, 50° C.”, “2×SSC, 0.1% SDS, 42° C.”, “1×SSC, 0.1% SDS, 37° C.”, more stringent condition can include conditions of “2×SSC, 0.1% SDS, 65° C.”, “0.5×SSC, 0.1% SDS, 42° C.” and “0.2×SSC, 0.1% SDS, 65° C.”.

Those skilled in the art can suitably obtain a protein functionally equivalent to the above proteins among the above proteins having a high homology by using a method of measuring an activity of synthesizing sphingomyelin. Specific methods for measuring the activity are illustratively described in the Examples. In addition, those skilled in the art can suitably obtain an endogenous gene in other organism which corresponds to the gene based on the base sequence of the gene. Here, in the present specification, protein and gene in an organism other than a human which corresponds to the above protein and gene, or protein and gene functionally equivalent to the above protein and gene are, in sometimes, merely described with the above names.

As a method of inhibiting the expression of a specific endogenous gene such as SMS2, methods using an antisense technique are well known to those skilled in the art. As an action that an antisense nucleic acid inhibits the expression of a target gene, a plurality of factors as follows exist. That is, inhibition of starting transcription by triplex formation, inhibition of transcription by forming a hybrid with a portion wherein the opened loop structure is locally made by an RNA polymerase, inhibition of transcription by forming a hybrid with an RNA which is being synthesized, inhibition of splicing by hybrid formation at the junction of an intron and an exon, inhibition of splicing by forming a hybrid with a spliceosome forming site, inhibition of translocation from nucleus to cytoplasm by hybrid formation with mRNA, inhibition of splicing by forming a hybrid with a capping site or poly (A) addition site, inhibition of starting translation by forming a hybrid with translation initiator-binding site, inhibition of translation by forming a hybrid with the ribosome binding site near start codon, inhibition of elongation of peptide chain by forming a hybrid with translated region or polysome binding site of mRNA, and inhibition of gene expression by forming a hybrid with nucleic acid-protein interacting site, or the like. As explained above, an antisense nucleic acid inhibits the expression of a target gene by inhibiting various processes such as transcription, splicing or translation (Hirashima and Inoue, Shin Seikagaku Jikken Koza [New Lectures on Biochemical Experiment] 2, Kakusan. [Nucleic acid] IV, Idenshi no Fukusei to Hatsugen [Gene Replication and Expression], The Japanese Biochemical Society, ed., Tokyo Kagaku Dojin, 1993, 319-347).

The antisense nucleic acid used in the present invention can inhibit the expression and/or function of the gene encoding SMS2 described above via any actions above. As one embodiment, if an antisense sequence is designed in which it is complementary to the untranslated region near 5′ end of mRNA of the gene encoding SMS2 described above, it is considered to be effective in inhibiting the translation of the gene. In addition, a sequence complementary to the coding region or the 3′ untranslated region can also be used. As such, a nucleic acid comprising an antisense sequence of not only the translated regions of the gene encoding the above SMS2 but also the untranslated regions is also encompassed by the antisense nucleic acid used in the present invention. An antisense nucleic acid used is liked downstream of a suitable promoter and is preferably liked, 3′ thereto, to a sequence comprising a transcription termination signal. A nucleic acid thus prepared can be transformed into a desired animal (cell) by using known methods. The sequence of an antisense nucleic acid is desirably a sequence complementary to a gene encoding an endogenous SMS2 in which the animal (cell) to be transformed has or part thereof, but are not necessarily completely complement as long as it can effectively suppress the expression of the gene. The transcribed RNA is preferably 90% or more, most preferably 95% or more complementary to the transcript of a target gene. In order to effectively inhibit the expression of a target gene by using an antisense nucleic acid, it is preferably that the length of the antisense nucleic acid is at least 12 bases or more and less than 25 bases, but the antisense nucleic acid of the present invention is not necessarily limited to such length and can be, for example, 11 bases or less, 100 bases or more, or 500 bases or more. The antisense nucleic acid can be composed of DNA only, but can comprise nucleic acids other than DNA, for example, a locked nucleic acid (LNA). As an embodiment, the antisense nucleic acid used in the present invention can be an antisense nucleic acid comprising a locked nucleic acid (LNA) at the 5′ end and an LNA at the 3′ end. Such an LNA-containing antisense nucleic acid can include, but not limited to, SEQ ID NOs: 29 to 40 or the like. In addition, in an embodiment of the present invention which uses an antisense nucleic acid, using, for example, the method described in Hirashima and Inoue, Shin Seikagaku Jikken Koza [New Lectures on Biochemical Experiment] 2, Kakusan [Nucleic acid] IV, Idenshi no Fukusei to Hatsugen [Gene Replication and Expression], The Japanese Biochemical Society, ed., Tokyo Kagaku Dojin, 1993, 319-347, based on the nucleic acid sequences of SMS2 set forth in SEQ ID NO: 87 and 88, antisense sequences can be designed. As sequences that can be referred to, SEQ ID NO: 87 and 88 or the like can be used, but not limited thereto. For example, those such as SEQ ID NOs: 29 to 40 are preferably used, but not limited thereto. These antisense nucleic acids can be confirmed for the effect of the antisense of the present invention by a method publicly known in the subject field which uses mice, cells or the like.

The inhibition of the expression of SMS2 can be carried out by utilizing a ribozyme or a DNA encoding a ribozyme. A ribozyme refers to an RNA molecule having a catalytic activity. Various ribozymes have various activities. Among them, a study focusing on ribozyme as an enzyme that cleaves RNAs made it possible to design a ribozyme that site-specifically cleaves RNAs. Some ribozymes are 400 nucleotides or more such as Group I intron type or M1 RNA included in RNase P, others called hammer head type and hairpin type have about 40 nucleotides of activity domain (Makoto Koizumi and Eiko Otsuka, Tanpakushitsu Kakusan Koso [Protein, Nucleic Acid and Enzyme], 1990, 35, 2191).

For example, the autocleaving domain of a hammerhead type ribozyme cleaves 3′ of C15 of a sequence Gl3U14C15, and the base-paring of U14 and A9 is important for the activity and it was demonstrated that Al5 or U15 instead of C15 can be cleaved (Koizumi, M. et al., FEBS Lett, 1988, 228, 228). If a ribozyme whose substrate-binding site is complementary to an RNA sequence near its target site is designed, an RNA-cleaving ribozyme which recognizes sequences UC, UU or UA in the target RNA like a restriction enzyme can be made (Koizumi, M. et al., FEBS Lett, 1988, 239, 285. Mokoto Koizumi and Eiko Otsuka, TanpakushitsuKakusanKoso [Protein, Nucleic Acid and Enzyme], 1990, 35, 2191., Koizumi, M. et al., Nucl Acids Res, 1989, 17, 7059).

Furthermore, a hairpin type ribozyme is also useful for the purpose of the present invention. The ribozyme is found in, for example, the minus strand of satellite RNA of tobacco ringspot virus (Buzayan, J M., Nature, 1986, 323, 349). It was demonstrated that a target specific RNA cleaving ribozyme can be also made from a hairpin type ribozyme (Kikuchi, Y. & Sasaki, N., Nucl Acids Res, 1991, 19, 6751, Yo Kikuchi, Chemistry and Biology, 1992, 30, 112). As described above, by specifically cleaving the transcript of a gene encoding SMS2 with a ribozyme, the expression of the gene can be inhibited.

Furthermore, the suppression of the expression of an endogenous gene such as SMS2 can also be carried out by RNA interference (abbreviated as “RNAi”, hereinafter) with a double stranded RNA having a sequence identical or similar to its target gene sequence. RNAi is currently a noted technique wherein when a double stranded RNA (dsRNA) is directly taken into a cell, the expression of a gene having a sequence homologous to the dsRNA is suppressed. In a mammalian cell, it is possible to induce RNAi by using a short chain dsRNA (siRNA). RNAi has many advantages over a knockout mouse that the effect is stable, experiment for it is easy, the cost is reasonable or the like. For siRNA, it is also described in detail in other positions herein.

As described above, those skilled in the art can suitably produce the “siRNA” of the present invention based on the base sequences of the genes encoding SMS2 described above which the double stranded RNA targets. As an example, a sense strand of the duplex RNA portion includes, but not limited to, SEQ ID NOs: 1, 3, 5, 7, 9, 11, 13, 15, 17, 21, 23, 25, 27 or the like. It can be suitably carried out by those skilled in the art within the scope of usual trial to select any contiguous RNA region of the mRNA that is a transcript of SMS2 sequence and then make a double stranded RNA corresponding to the region. It can be also suitably carried out by those skilled in the art with known methods to select an siRNA sequence having a stronger RNAi effect among mRNA sequences that are the transcript of the sequence. In addition, if one strand is known, those skilled can also readily know the base sequence of the other strand (a complementary chain). Those skilled in the art can suitably make an siRNA by using a commercially available nucleic acid synthesizer. Alternatively, for the synthesis of a desired RNA, a usual contract service for synthesis can be used.

Therefore, in one embodiment, the present invention is an siRNA of SMS2 (for example, SEQ ID NO: 79 or 80 which are the full length sequences of SMS2). Such siRNA specifically includes, but not limited to, siRNA of any one selected from the group consisting of the following (a) to (p), which are siRNAs based on the sequences which the present inventors have originally designed:

(a) an siRNA wherein one strand of the duplex RNA portion is a base sequence set forth by SEQ ID NO: 1 and the other strand of the duplex RNA portion is a base sequence set forth by SEQ ID NO: 2 <SMS2-i6>;
(b) an siRNA wherein one strand of the duplex RNA portion is a base sequence set forth by SEQ ID NO: 3 and the other strand of the duplex RNA portion is a base sequence set forth by SEQ ID NO: 4 <SMS2-i7>;
(c) an siRNA wherein one strand of the duplex RNA portion is a base sequence set forth by SEQ ID NO: 5 and the other strand of the duplex RNA portion is a base sequence set forth by SEQ ID NO: 6 <SMS2-i8>;
(d) an siRNA wherein one strand of the duplex RNA portion is a base sequence set forth by SEQ ID NO: 7 and the other strand of the duplex RNA portion is a base sequence set forth by SEQ ID NO: 8 <SMS2-i104>;
(e) an siRNA wherein one strand of the duplex RNA portion is a base sequence set forth by SEQ ID NO: 9 and the other strand of the duplex RNA portion is a base sequence set forth by SEQ ID NO: 10 <SMS2-i105>;
(f) an siRNA wherein one strand of the duplex RNA portion is a base sequence set forth by SEQ ID NO: 11 and the other strand of the duplex RNA portion is a base sequence set forth by SEQ ID NO: 12 <SMS2-i106>;
(g) an siRNA wherein one strand of the duplex RNA portion is a base sequence set forth by SEQ ID NO: 13 and the other strand of the duplex RNA portion is a base sequence set forth by SEQ ID NO: 14 <SMS2-i107>;
(h) an siRNA wherein one strand of the duplex RNA portion is a base sequence set forth by SEQ ID NO: 15 and the other strand of the duplex RNA portion is a base sequence set forth by SEQ ID NO: 16 <SMS2-i108>;
(i) an siRNA wherein one strand of the duplex RNA portion is a base sequence set forth by SEQ ID NO: 17 and the other strand of the duplex RNA portion is a base sequence set forth by SEQ ID NO: 18 <SMS2-i109>;
(j) an siRNA wherein one strand of the duplex RNA portion is a base sequence set forth by SEQ ID NO: 21 and the other strand of the duplex RNA portion is a base sequence set forth by SEQ ID NO: 22 <SMS2-i3>;
(k) an siRNA wherein one strand of the duplex RNA portion is a base sequence set forth by SEQ ID NO: 23 and the other strand of the duplex RNA portion is a base sequence set forth by SEQ ID NO: 24 <SMS2-i11>;
(l) an siRNA wherein one strand of the duplex RNA portion is a base sequence set forth by SEQ ID NO: 39 and the other strand of the duplex RNA portion is a base sequence set forth by SEQ ID NO: 40 <SMS2-i1>;
(m) an siRNA wherein one strand of the duplex RNA portion is a base sequence set forth by SEQ ID NO: 41 and the other strand of the duplex RNA portion is a base sequence set forth by SEQ ID NO: 42 <SMS2-i2>;
(n) an siRNA wherein one strand of the duplex RNA portion is a base sequence set forth by SEQ ID NO: 45 and the other strand of the duplex RNA portion is a base sequence set forth by SEQ ID NO: 46 <SMS2-i5>; and
(p) an siRNA of any of (a) to (n), wherein one to several nucleotides are added, inserted, deleted or substituted in one or both of the base sequences, and having an activity of suppressing the expression of SMS2.

The siRNA in the present invention is not necessarily a pair of double stranded RNA to its target sequence, but can be a mixture of multiple pairs of double stranded RNA toward regions comprising the target sequence. Here, siRNA as a nucleic acid mixture corresponding to a target sequence can be suitably made by those skilled in the art with a commercially available nucleic acid synthesizer and DICER enzyme. Alternatively, for the synthesis of a desired RNA, a usual contract service for synthesis can be used. Here, siRNA of the present invention encompasses the so-called “cocktail siRNA”. Furthermore, not all nucleotides in the siRNA in the present invention are necessarily ribonucleotides (RNAs). That is, in the present invention, one or a plurality of ribonucleotide(s) that constitutes siRNA can be a corresponding deoxyribonucleotide. The “corresponding” refers to being same kind of base (adenine, guanine, cytosine, thymine (uracil)) with different sugar structures. For example, a corresponding deoxyribonucleotide to a ribonucleotide having adenine refers to a deoxyribonucleotide having adenine. In addition, the above “plurality” refers to, but not specifically limited to, preferably as small a number as about 2 to 5.

Furthermore, a DNA (a vector) capable of expressing the RNA of the present invention can also be encompassed by a preferred embodiment of the nucleic acid of the present invention that is capable of suppressing the expression of SMS2. For example, a DNA (a vector) capable of expressing the double stranded RNA of the present invention is a DNA having a structure wherein a DNA encoding one strand of the double stranded RNA and a DNA encoding the other strand of the double stranded RNA are attracted to the promoters so that each of the DNA can be expressed. Those skilled in the art can suitably make the DNA of the present invention by general genetic engineering techniques. More specifically, the expression vector of the present invention can be made by suitably inserting a DNA encoding the RNA of the present invention into various known expression vectors.

### (General Techniques)

Techniques of manufacturing a medical device, techniques of formulation, techniques of microfabrication, molecular biological methods, biochemical methods, microbiological methods, saccharide chain related methods used herein are well known and commonly used in the art, and described in for example, Maniatis, T. et al. (1989) Molecular Cloning: A Laboratory Manual, Cold Spring Harbor and 3rd Ed. (2001); Ausubel, F. M., et al. eds, Current Protocols in Molecular Biology, John Wiley & Sons Inc., NY, 10158 (2000); Innis, M. A. (1990) PCR Protocols: A Guide to Methods and Applications, Academic Press; Innis, M. A. et al. (1995) PCR Strategies, Academic Press; Sninsky, J. J. et al. (1999) PCR Applications: Protocols for Functional Genomics, Academic Press; Gait, M. J. (1985) Oligonucleotide Synthesis: A Practical Approach, IRL Press; Gait, M. J. (1990) Oligonucleotide Synthesis: A Practical Approach, IRL Press; Eckstein, F. (1991) Oligonucleotides and Analogues: A Practical Approac, IRL Press; Adams, R. L. et al. (1992) The Biochemistry of the Nucleic Acids, Chapman & Hall; Shabarova, Z. et al. (1994) Advanced Organic Chemistry of Nucleic Acids, Weinheim; Blackburn, G. M. et al. (1996) Nucleic Acids in Chemistry and Biology, Oxford University Press; Hermanson, G. T. (1996) Bioconjugate Techniques, Academic Press; Method in Enzymology 230, 242, 247, Academic Press, 1994; Bessatsu Jikken Igaku [Experimental Medicine, Supplemental Volume], Idenshi Donyu Oyobi Hatsugen Kaiseki Jikken Ho [Experimental Methods for Transgenesis & Expression Analysis], Yodosha, 1997 or the like, relevant portions (which may be all) of which are incorporated herein by reference.

Methods of culture used in the present invention are described and supported in for example, Dobutsu Baiyo Saibo Manyuaru [Animal Cultured Cell Manual], Seno et al. ed., Kyoritsu Syuppan, 1993 or the like, the entire description of which are incorporated herein.

Hereinabove, the present invention has been described by showing preferred embodiments for ease of understanding. While the present invention is described hereinbelow based on examples, the above description and the examples below are provided for illustrative purposes only, but not provided for a purpose for limiting the present invention. Therefore, the scope of the present invention is not limited by embodiments or examples that are specifically described herein, but limited only by the Claims.

### EXAMPLES

The animals used in the following Examples were handled in compliance with the standards set forth by the Hokkaido University.

### (Materials and Methods)

### (Antibodies and Reagents)

Primary antibodies were obtained from the following suppliers: murine monoclonal antibodies against Alix, binding immunoglobulin proteins (BiP), GM130 (BD Transduction Laboratories) and Aβ (6E10, Signet); and rabbit polyclonal antibodies against Tsg-101 (Santa Cruz Biotechnology) and Aβ oligomers (A11, Invitrogen). Secondary antibodies were obtained from GE Healthcare. Thioflavin T (ThT), cholera toxin subunit B (CTB), HRP-conjugated CTB, annexinV (AV), imipramine, GW4869, D609 and bacterial SMase (Staphylococcus aureus) were obtained from Sigma. AlexaFluor594-conjugated CTB, AlexaFluor488-conjugated AV, and LysoTracker Green DND-26 and LysoTracker BlueDND-22 were purchased from Invitrogen. N-hexanoyl-D-erythro-sphingosine (C6-ceramide, d18:⅙: 0) was obtained from Avanti Polar Lipids. For synthetic Aβ peptides, human Aβ1-40 (Aβ40, Peptide Institute), Aβ1-42 (Aβ42, Peptide Institute) and FAN-conjugated human Aβ42 (AnaSpec) were used.

### (Cell Cultures)

Murine neuroblastoma Neuro2a (also referred to as N2a herein) was maintained in Dulbecco's Modified Eagle's Medium (Invitrogen) supplemented with 10% fetal bovine serum. Murine microglial cell line BV-2 was purchased from the National Cancer Institute (Instituto Nazionale per la Ricerca sul Cancro, Genova, Italy) and cultured in RPMI1640 (Invitrogen) supplemented with 10% fetal bovine serum and L-glutamine.

A primary culture of nerve cells was prepared from the cerebral cortex of the brain of a 15 day old embryonic mouse according to the method of Levi et al. (Levi G et al., “A Dissection and Tissue Culture Manual of Nervous System”, Alan R Liss, Inc., New York, N.Y. (1989)). Briefly stated, nerve cells were prepared from the isolated cerebral cortex by using a cell dispersion solution (Sumitomo Bakelite Co., Ltd). The cells were then plated onto a polyethyleneimine (PEI)-coated dish at a density of 5.0×10⁵ cells/cm² and cultured in a Neurobasal medium (Invitrogen) comprising 25 mM KCl, 2 mM glutamine and B27 supplement (Invitrogen). Primary cultured microglia prepared from a newborn rat were purchased from Sumitomo Bakelite Co., Ltd. and maintained in a microglial culture medium according to the manufacturer's protocol.

### (Isolation of Exosome)

As previously described (Thery C et al., Curr Protoc Cell Biol Chapter: Unit 3.22 (2006)), exosomes were prepared from culture supernatants of N2a and murine primary cultured cortical nerve cells. The culture medium was replaced with a serum-free medium on the day before the preparation of exosomes. The cell culture supernatants were collected after 24 hours and sequentially centrifuged at 3,000×g for 10 minutes, at 4,000×g for 10 minutes, and at 10,000×g for 30 minutes to remove cells, dead cells, and cell fragments, and then spun down again at 100,000×g for one hour to obtain exosomes as pellets.

For sucrose gradient analysis, exosome pellets (corresponding to the amount from 5×10⁷ cells) were loaded into 10 ml of sucrose gradient (0.25-2.3 M sucrose in 10 ml of 20 mM HEPES) and centrifuged at 100,000×g for 18 hours. After the centrifugation, a small amount (1 ml) was collected, diluted with 20 mM HEPES, and precipitated by centrifugation at 100,000×g for one hour. The obtained pellet was resuspended in PBS and subjected to Western blot.

### (Electronic Microscopy)

100,000×g pellets purified from culture supernatants of N2a cells and primary cultured cortical nerve cells were resuspended in TBS and put on a collodion-covered grid. In addition, the suspension was negatively stained with 2% phosphotungstic acid (Nisshin EM Corporation). Photomicrographs were captured using an HD-2000 scanning transmission electron microscope (Hitachi High-Technologies Corporation).

### (Seed-Free Aβ Preparation)

A seed-free Aβ solution was prepared essentially according to the previous report (Naiki H et al., Methods Enzymol (1999) 309: 305-318). Briefly stated, synthetic Aβ40 and Aβ42 were dissolved in a 0.02% ammonium solution at 500 μM and 300 μM, respectively. In order to remove undissolved Aβ aggregates that can act as pre-existing seeds, the prepared solution was centrifuged at 540,000×g for three hours at 4° C. The obtained supernatant was collected and stored at −80° C. until use.

### (Thioflavin T Test)

Seed free Aβ (25 μM) was incubated in 100 μl of TBS comprising 0 μl, 1 μl, or 10 μl of exosome solution at 37° C. 1 μl of exosome solution was collected from culture supernatants of 1×10⁶ cells. As described in the prior art document (Naiki H et al., Methods Enzymol (1999)309:305-318), fluorescence intensity of ThT in the mixture was measured using an Appliskan spectrofluorophotometer (Thermo Fisher Scientific). The optimal fluorescence intensities of amyloid fibrils were measured at an excitation wavelength of 446 nm and fluorescent wavelength of 490 nm by using a reaction mixture comprising 5 μM ThT and 50 mM glycine/NaOH with pH of 8.5.

### (Dot Blot)

Seed-free Aβ42 (25 μM) was incubated in 100 μl of TBS with or without exosomes (100,000×g pellet) over the indicated time at 37° C., and the solution was dotted onto a nitrocellulose membrane. The membrane was probed with an antibody against Aβ oligomer (A11) and an antibody against Aβ (6E10), and subsequently probed with an HRP-conjugated secondary antibody. A combination of an ECL Plus kit (GE Healthcare) and an LAS4000 (Fujifilm Corporation) was used to detect and analyze chemiluminescence.

### (Toxicity Assay)

Seed-free Aβ (25 μM) was reacted with or without exosomes for five hours at 37° C. in 100 μl of Neurobasal medium supplemented with 25 mM KCl, 2 mM glutamine and B27 additive. The pre-incubated mixture was subsequently added to primary cultured cortical nerve cells that had been plated on a 24-well plate (DIV) and incubated for 24 hours. The cell viability was measured by using a WST-1 (Dojindo).

### (Drug Treatment)

Treatment with imipramine (10 μM), GW4869 (10 μM), D609 (50 μM) C6-ceramide (50 μM) or bacterial SMase (100 μU/ml) was performed for 24 hours in a serum-free medium.

(siRNA Delivery and Transfection)

For RNA-mediated interference (RNAi) experiments, the inventors used Stealth RNAi™ siRNA (Invitrogen) with the following sequences:

For SMS1
(sense; SEQ ID NO: 41)
5′-AUACAUUGUAAUACACCGAUACAGG-3′
and
(antisense; SEQ ID NO: 42)
5′-CCUGUAUCGGUGUAUUACAAUGUAU-3′;
For SMS2:
(sense; SEQ ID NO: 43)
5′-AUACAUAGUUAUACAGCGAUACAGG-3′
and
(antisense; SEQ ID NO: 44)
5′-CCUGUAUCGCUGUAUAACUAUGUAU-3′;
For aSMase:
(sense; SEQ ID NO: 45)
5′-AUUGGUUUCCCUUUAUGAAGGGAGG-3′
and
(antisense; SEQ ID NO: 46)
5′-CCUCCCUUCAUAAAGGGAAACCAAU-3′;
For nSMase1:
(sense; SEQ ID NO: 47)
5′-AAUAGAACCACAUCUGCAUUCUUGG-3′
and
(antisense; SEQ ID NO: 48)
5′-CCAAGAAUGCAGAUGUGGUUCUAUU-3′;
For nSMase2:
(sense; SEQ ID NO: 49)
5′-AAUCGAUGUAGAUCUUGAUCUGAGG-3′
and
(antisense; SEQ ID NO: 50)
5′-CCUCAGAUCAAGAUCUACAUCGAUU-3′.

Stealth™ Control RNA was obtained from Invitrogen SiRNA was delivered according to the manufacturer's protocol by using a Lipofectamine™ transfection reagent (Invitrogen).

The cDNA of a human amyloid precursor protein (APP) 770 was amplified from human brain cDNA (Clontech) by PCR that uses the following primers:

(sense; SEQ ID NO: 51)
5′-ATGCTGCCCGGTTTGG-3′
and
(antisense; SEQ ID NO: 52)
5′-CTACTTCTGCATCTGCTCAAAGAACTTG-3′.

The cDNA was then cloned to a pENTRTMD-TOPO vector (Invitrogen) to ultimately construct p3×FLAG-APP770 by using the Gateway™ recombination system as described previously (Mitsutake S et al., J Biol Chem (2011) 286: 28544-28555). Transient transfection was performed using a Lipofectamine-2000 (Invitrogen) according to the manufacturer's protocol.

### (Measurement of Exosome Release)

Exosome pellets purified from cultures of 5×10⁶ cells were solubilized with Laemmli buffer (Laemmli U K, Nature (1970) 227: 680-685) and subjected to SDS-PAGE and Western blotting. Blots were probed with a primary antibody and then probed with an HRP-conjugated secondary antibody. Bands were detected and analyzed using a combination of an ECL Plus kit (GE Healthcare) and an LAS4000 (Fujifilm Corporation).

### (Fluorescence Staining and Internalization Assay)

Exosomes were fluorescently stained with red dye PKH26 (Sigma) according to the manufacturer's protocol (Morelli A E et al., Blood (2004) 104: 3257-3266). Briefly stated, exosomes (100,000×g pellet) were resuspended in diluent C and stained with PKH26 for 5 minutes, and the reaction was then stopped with 1% bovine serum albumin. The labeled exosomes were precipitated again by ultracentrifugation at 100,000×g for one hour. The PKH26-labeled exosomes were exposed to BV-2 cells on chamber slides (Nunc) over the indicated time under serum-free conditions. For inhibition experiments, the labeled exosomes were pretreated with AV or CTB (0, 0.5, or 1 μM) at 37° C. for 15 minutes. To observe Aβ transfer into BV-2 cells by exosomes, the labeled exosomes were pre-incubated for 5 hours at 37° C. with 25 μM of fluorescence (FAM)-labeled Aβ42. The cells were then fixed with 4% paraformaldehyde, and confocal images were obtained using a Fluoview™ FV10i (Olympus). Fluorescence intensities were analyzed with ImageJ (http://rsbweb.nih.gov/ij/).

### (Aβ Measurement)

Aβ40 and Aβ42 levels were measured by using a sandwich enzyme-linked immunosorbent assay (ELISA) kit from Wako. Aggregated Aβs both in medium and cells were solubilized with 4 M guanidine-HCl buffer for 2 hours at room temperature and then were subjected to ELISA. All samples were measured in duplicates.

### (Transwell Experiment)

N2a cells were cultured on 24-well plate inserts (0.5 μm pore, Costar) at 5×10⁵ cells/cm² and then transfected with the APP770 plasmid simultaneously with siRNA for nSMase2 or SMS2 by using a Lipofectamine-2000. After 24 hours from the transfection, inserts were placed onto wells containing BV-2 cells. After an additional 24 hours of incubation, Aβ levels in the medium were measured by ELISA. Intracellular Aβ levels in BV-2 cells were also measured using ELISA after solubilization in guanidine HCl buffer as described above.

### (Results)

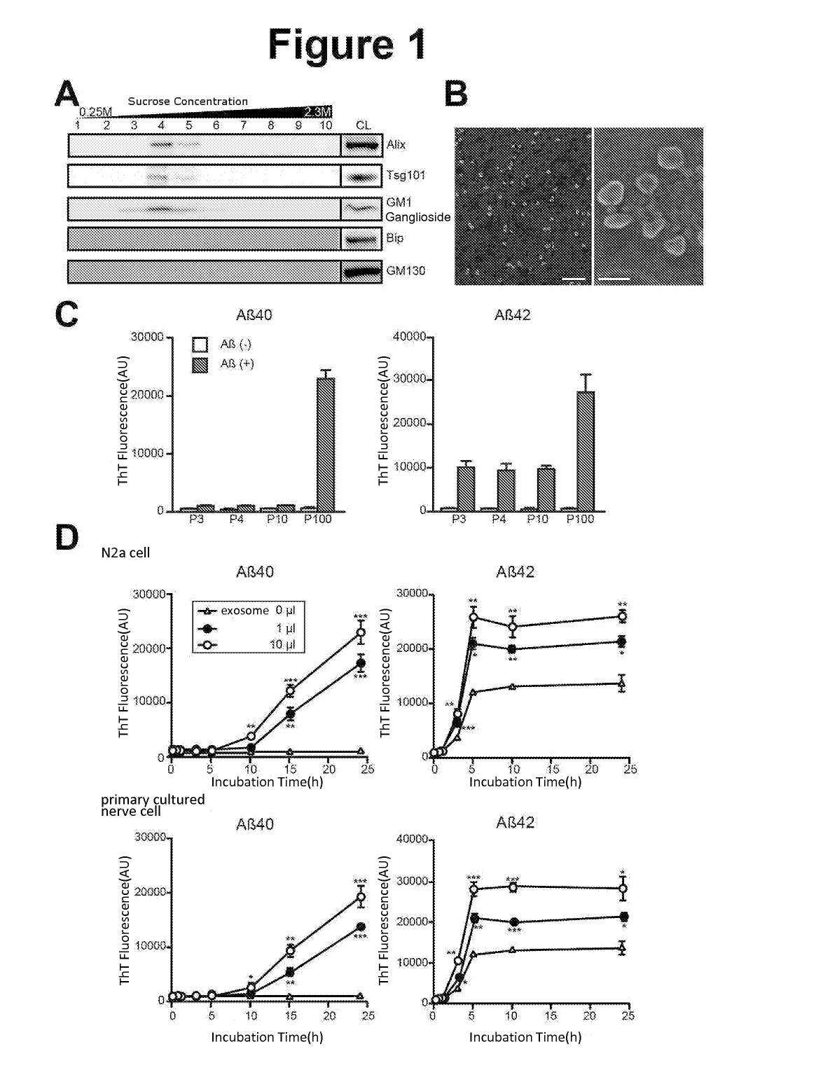

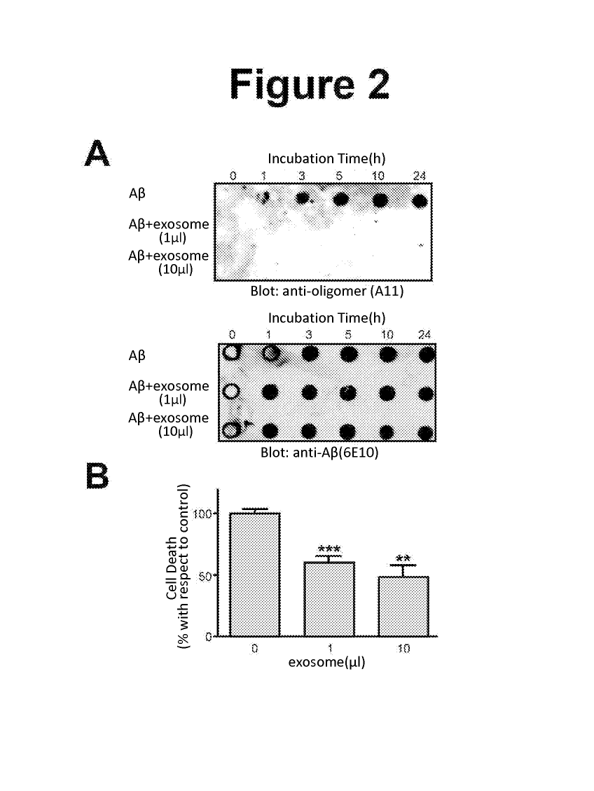

### (Experimental Results Related to FIGS. 1-2)

### (Nerve Cell-Derived Exosomes Drive Aβ to Form Amyloid Fibrils)

Culture media of N2a cells and primary cultured cortical nerve cells were subjected to sequential centrifugation steps with increasing centrifugal forces, ultimately obtaining 100,000×g pellets. Electron microscopy analysis revealed that the pellet collected from the N2a cultures was mainly comprised of small membrane vesicles with a diameter of about 40 to 100 nm (FIG. 1B), similar to previously described exosomes prepared from other cell cultures (Thery C et al., Nat Rev Immunol (2002) 2: 569-579). In a continuous sucrose density gradient, exosomal proteins, Alix and Tsg101, were detected in fractions 4 and 5 (corresponding to sucrose densities of 1.12 and 1.16 g/ml, FIG. 1A), as previously reported (Simons M et al., Curr Opin Cell Biol (2009) 21: 575-581). In addition, exosomes are reportedly rich in proteins and lipids and are bound to lipid microdomains (deGassart A et al., Blood (2003) 102: 4336-4344). Ganglioside GM1 (GM1), a glycosphingolipid abundant in lipid microdomains, was also detected in high concentrations in the same fractions as Alix and Tsg101. In contrast, BiP and GM130, marker proteins for endoplasmic reticulum (ER) and for Golgi apparatus, respectively, were not observed in the 100,000×g pellets. The pellets collected from the primary-cultured nerve cell cultures also had similar sizes and densities as those of membrane vesicles having Alix, Tsg101, and GM1 (data not shown). These data confirm that the 100,000×g pellets are mainly comprised of exosomes and demonstrate that the exosomes are secreted from N2a and primary cultured cortical nerve cells in a constitutive manner.

To examine the effect of nerve cell-derived exosomes on Aβ conformational change, the inventors mixed the pellets obtained from the sequential centrifugations (P3, P4, P10, and P100) with soluble Aβ40 and Aβ42, two major species of Aβ, and incubated the mixture at 37° C. for 24 hours. The amount of amyloid fibrils was then determined using Thioflavin T (ThT). As a result, ThT fluorescence intensities were significantly enhanced only by the P100 exosome fraction (FIG. 1C). FIG. 1D shows the elapsed time in Aβ amyloid generation under the presence or absence of exosomes. Both N2a—and primary cultured nerve cell-derived exosomes significantly accelerated fibril formation of Aβ40 and Aβ42 in a time-dependent manner. In cases of Aβ42, the amount of amyloid Aβ significantly increased after reaching a plateau phase. In addition, in order to investigate the effect of exosomes on the formation of oligomeric Aβ, mixtures of Aβ with or without N2a-derived exosomes were subjected to a dot blot analysis with an anti-oligomer antibody A11 (FIG. 2A). In the absence of exosomes, oligomeric Aβ was formed just after one hour of incubation at 37° C. In the presence of exosomes, however, oligomeric Aβ was not detected in the incubation mixture in 24 hours of incubation. Accumulated evidence indicates that neurodegeneration and synaptic impairment in AD pathogenesis are directly caused by soluble Aβ oligomers (Selkoe D J, Science (2002) 298: 789-791; Haass C et al., Nat RevMol Cell Biol (2007) δ: 101-112). Indeed, an Aβ solution that had been pre-incubated at 37° C. for 5 h without exosomes induced remarkable cell death in primary-cultured nerve cells (FIG. 2B). In contrast, addition of exosomes to the incubation mixtures dramatically prevented nerve cell death. These findings suggest that nerve cell-derived exosomes promote rapid conformational change of Aβ into nontoxic amyloid fibrils on the surface thereof, resulting in a decline in the spontaneous formation of toxic oligomeric species.

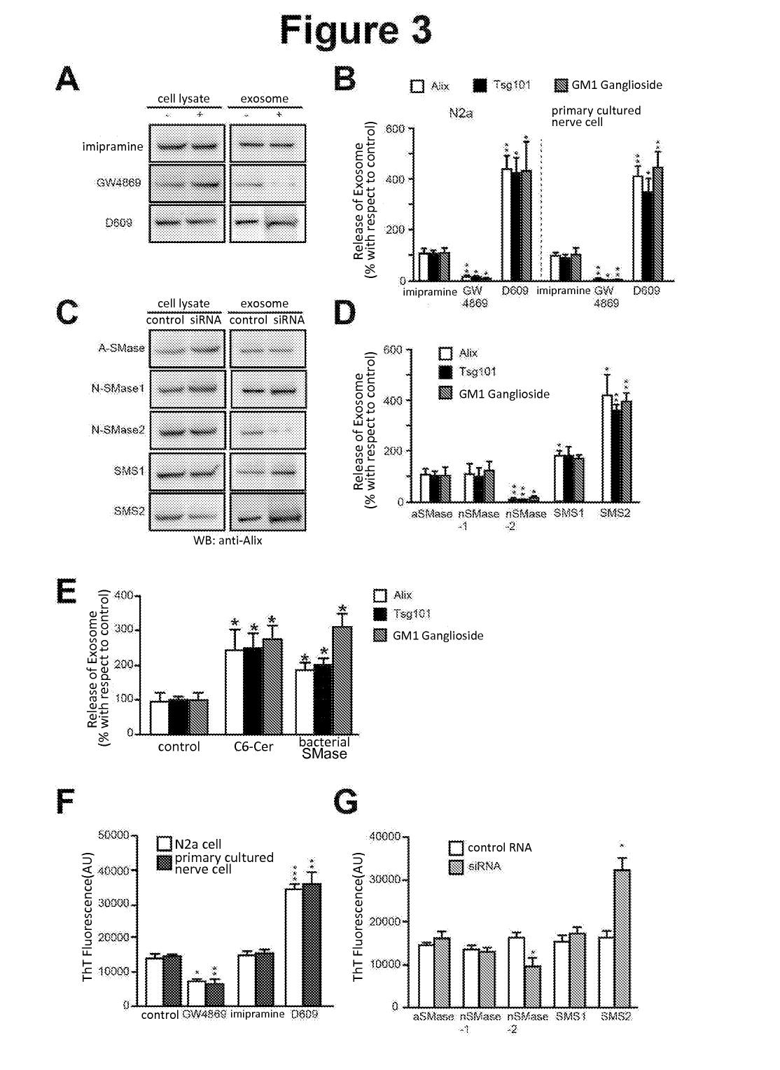

### (Experimental Results Related to FIG. 3)

### (Sphingolipid Metabolism is Involved in Exosome Secretion and Aβ Fibril Formation)

Biosynthesis of exosomes originates from the budding of intraluminal vesicles into multivesicular endosomes. Trajkovic et al. reported that sphingolipid ceramide triggers the intraluminal uptake and induces exosome release (Trajkovic K et al., Science (2008) 319: 1244-1247). To investigate whether nerve cell derived exosomes can be modulated by sphingolipid metabolism, the inventors first treated N2a cells and primary cultured nerve cells with a few inhibitors (GW4869, imipramine, and D609) for sphingolipid-metabolizing enzymes (FIGS. 3A and 3B). The levels of released exosomes were assessed by the exosomal markers, Alix, Tsg101, and GM1, in 100,000×g pellets. Treatment with an inhibitor of neutral sphingomyelinase (nSMase) GW4869, which converts sphingomyelin (SM) to ceramide (Cer), significantly decreased levels of released exosomes. This is in agreement with the previous report (Trajkovic K et al., Science (2008) 319: 1244-1247). In contrast, treatment with imipramine, which selectively inhibits acid sphingomyelinase (aSMase), did not change exosome release. D609 has been reported to inhibit sphingomyelin synthase (SMS), which catalyzes the opposite reaction to SMase, i.e., conversion of Cer into SM (Luberto C et al., J Biol CChem (1998) 273: 14550-14559). Predictably, significant enhancement in exosome secretion was exhibited due to treatment with D609. To further examine the role of sphingolipid metabolism in exosome secretion, the inventors employed an RNA interference approach to knock down the expression of endogenous SMases and SMSs in N2a cells. In agreement with the previous report (Traj kovic K et al., Science (2008) 319: 1244-1247), treatment with siRNA for nSMase2 reduced exosome release (FIGS. 3C and 3D). Meanwhile, treatment with siRNA against aSMase or nSMase1 did not change the release of exosomes. Conversely, an effect of significant increases in exosome secretion was exhibited from knockdown of both SMS1 and SMS2. A more evident increase in exosome release was exhibited, especially from SMS2 knockdown compared with SMS1 knockdown (ratio of amount of Alix compared with a control, 186.02±9.77% for SMS1 siRNA, 424.75±45.96% for SMS2 siRNA, FIG. 2D). These results indicate that Cer production affects exosome secretion. Indeed, exogenously added Cer and Cer production induced by exogenously added SMase significantly increased release levels of exosomes (FIG. 3E).

Next, the inventors investigated the role of sphingolipid metabolism on exosome-mediated Aβ fibrillogenesis. As stated above, the inventors collected exosomes from culture supernatants of cells that had been treated with an inhibitor or siRNA, and the inventors measured the ThT fluorescence in mixtures of exosomes and Aβ42 after five hours of incubation at 37° C. (FIGS. 3F and 3G). Exosomes purified from GW4869- or nSMase2-treated cultures significantly reduced Aβ fibrillogenesis, while exosomes purified from D609- or SMS2-treated cultures increased amyloid formation instead, compared with that in controls. From these data, the inventors consider the potential of exosomes to modulate Aβ fibril formation to be closely correlated with the amount thereof. In addition, release levels of exosomes can be modulated by enzyme activity associated with sphingolipid synthesis.

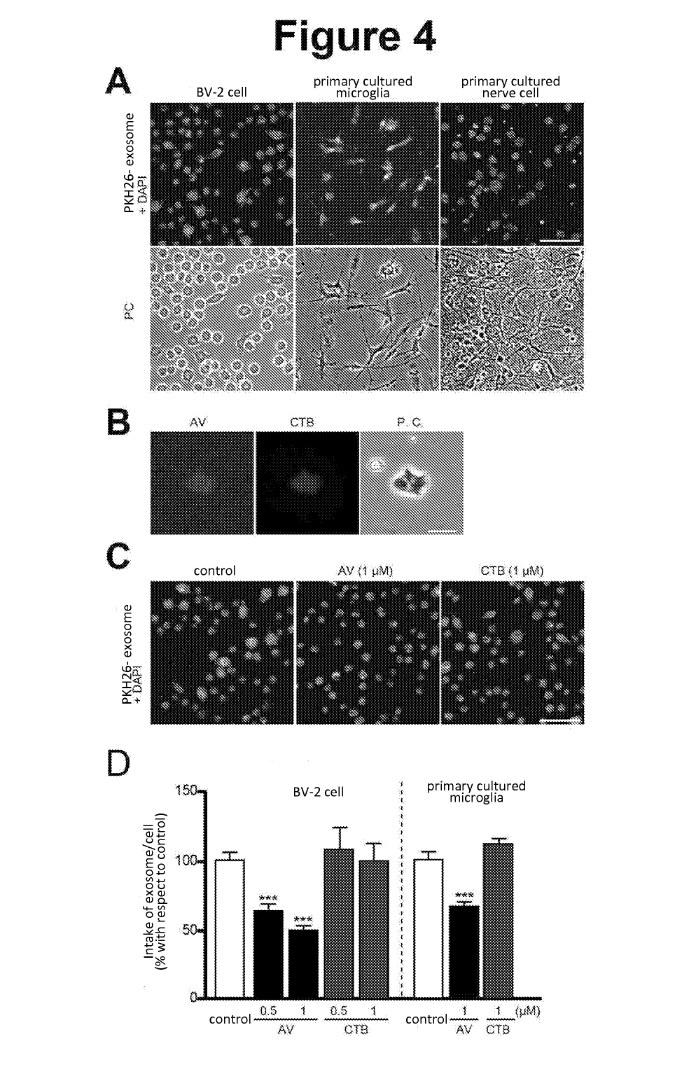

### (Experimental Results Related to FIG. 4)

### (Microglia Engulf Exosomes in a Phosphatidylserine (PS)—Dependent Manner)

Microglia are resident phagocytes in the central nervous system. It is now widely accepted that they are derived from macrophages and contribute to the removal of dead cells and cell fragments in the brain (Napoli I et al., Neuroscience (2009) 158: 1030-1038). Several reports have revealed that macrophages also take up exosomes secreted from several different cells to transduce inflammatory signals or remove the exosomes (Ransohoff R M, Nat Neurosci (2007) 10: 1507-1509). Recently, Fitzner et al. reported that oligodendrocyte-derived exosomes are specifically taken up by microglia in the brain (Fitzner D et al., J. Cell Sci (2011) 124: 447-458). To assess whether microglia also take up nerve cell derived exosomes, exosomes labeled with the fluorescent dye PKH26 were added to microglial cell line BV-2, primary cultured microglia, or primary cultured cortical nerve cells. After three hours of incubation with the labeled exosomes at 37° C., the cells were fixed, stained with DAPI, and analyzed with a confocal microscope. The inventors observed significant fluorescence in both BV-2 and primary cultured microglia (FIG. 4A). These results suggest that exosomes are efficiently internalized into microglia. In contrast, fluorescent signals were rarely detected in primary cultured nerve cells, further demonstrating the selective transfer of nerve cell-derived exosomes into microglia, which is in agreement with the previous report (Fitzner D et al., J Cell Sci (2011) 124: 447-458) using oligodendrocyte-derived exosomes. Various cells produce exosomes expressing phosphatidylserine (PS) on the surface thereof, and PS exposed on the outer leaflet of the plasma membrane is often used as a recognition signal for engulfment of apoptotic cells by macrophages and microglia (Miyanishi M et al, Nature (2007) 450: 435-439; Morelli A E et al., Blood (2004) 104: 3257-3266). The inventors, without permeabilizing an outer membrane, stained 100,000×g pellets with fluorescently labeled annexin V (AV) or cholera toxin B subunit (CTB), which specifically recognize PS and GM1, respectively. Significant fluorescence of both AV and CTB was observed (FIG. 4B), suggesting PS is located on the outer leaflet of N2a-derived exosomes. To further clarify the mechanism for exosome uptake by microglia, the inventors exposed exosomes pre-incubated with AV or CTB to microglial cultures. The inventors found that treatment with AV significantly suppressed uptake of exosomes into BV-2 cells or primary cultured microglia (FIGS. 4C and 4D). However, treatment of exosomes with CTB did not change CTB uptake. These results suggest that PS promotes recognition and internalization of nerve cell-derived exosomes by microglia.

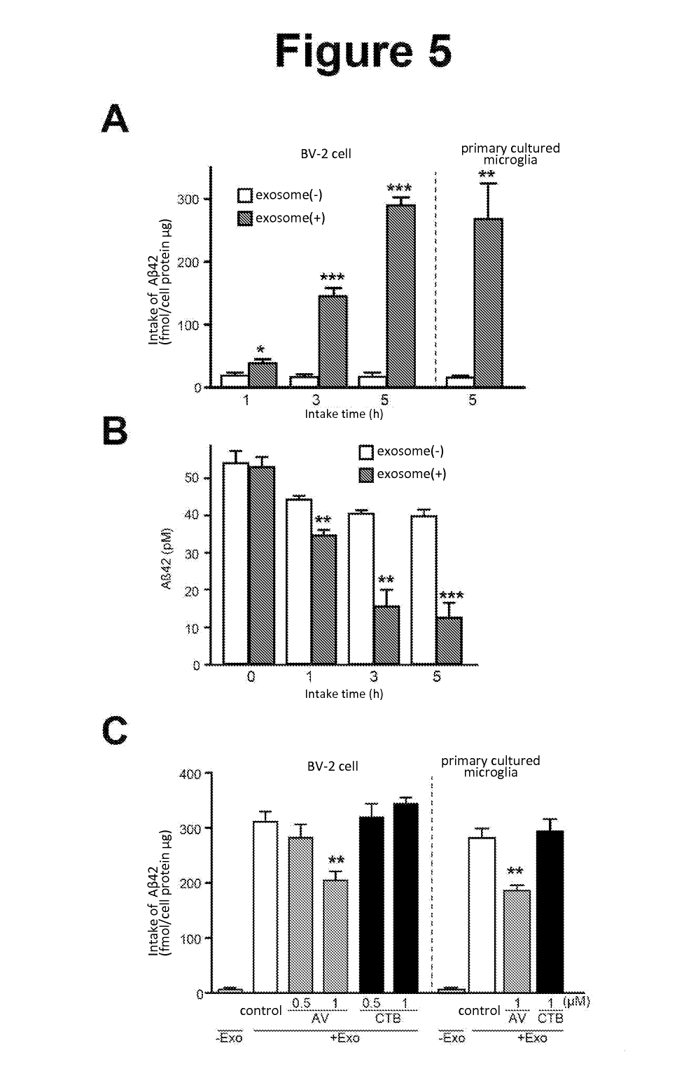

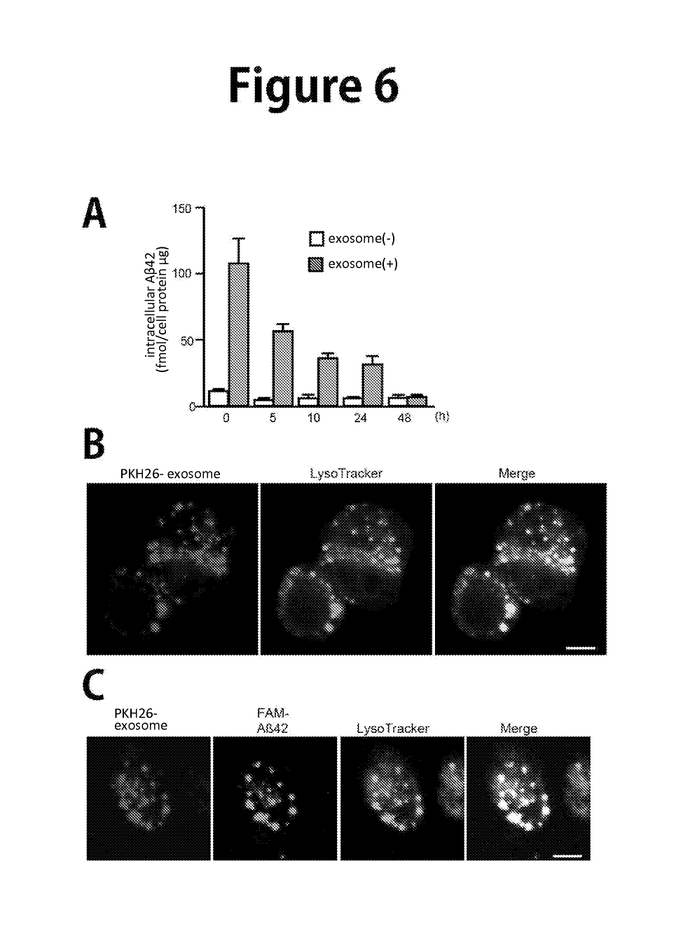

### (Experimental Results Related to FIGS. 5-6)

### (Exosomes Promote Aβ Clearance in Microglia)

Interaction between exosomes and Aβ results in accelerated Aβ fibril formation (FIGS. 1C and 1D), suggesting that Aβ amyloid fibrils accumulate to surround exosomes. Indeed, the exosomal marker Alix was observed to be concentrated in the senile plaque, an extracellular deposition of Aβ fibrils in AD brain (Rajendran L et al., Proc Natl Acad Sci USA (2006) 103: 11172-11177). Furthermore, the present application demonstrates that exosomes are taken up by microglia (FIG. 4A). Based on these findings, the inventors hypothesized that exosomes may have support transfer of Aβ amyloid into microglia to likely aid in Aβ degradation. To verify this hypothesis, the inventors added Aβ42, pre-incubated with or without exosomes, to BV-2 cells or primary cultured microglia. After incubating at 37° C. over the indicated time, intracellular and extracellular levels of Aβ42 were measured. As a result, both BV-2 and primary cultured microglia took up dramatically more Aβ in the presence of exosomes compared to a case of Aβ42 alone (FIG. 5A). Correspondingly, Aβ levels in the medium gradually decreased, with significant difference between Aβ levels in the presence of exosomes and in absence of exosomes (FIG. 5B). The inventors blocked PS on the outer surface of exosomes by AV to further investigate whether the prevention of exosome uptake may affect Aβ uptake into microglia. As shown in FIG. 5C, Aβ uptake was significantly suppressed only when exosomes had been pre-incubated with AV, but not when pre-incubated with CTB. These results suggest that exosomes can, at least partially, mediate Aβ uptake in a PS-dependent manner.

Next, to assess whether Aβ internalized with exosomes is degraded in microglia, the inventors exposed Aβ42, pre-incubated with or without exosomes, to BV-2 cells for three hours and washed the Aβ42 twice in culture medium. After additional culture time as shown, cells were collected and Aβ levels in the cell lysates were measured. The Aβ levels in the BV-2 cells gradually decreased in a time-dependent manner and were nearly depleted at 48 hours after washing (FIG. 6A). To gain insight into the degradation pathway of the internalized exosomes and Aβ, the inventors investigated their localization by staining with LysoTracker, a fluorescence marker of late endosome/lysosomes. The inventors incubated PKH26-labeled, N2a-derived exosomes with BV-2 cells for three hours at 37° C. Punctate fluorescence was observed in the cells and portions of the exosome fluorescence co-localized with lysosomal compartments (FIG. 6B). The inventors next added co-incubated mixture of FAM-Aβ42 and labeled exosomes to BV-2 cells. Together with exosome fluorescence, a signal of Aβ also co-localized with a LysoTracker signal (FIG. 6C). These data demonstrated that Aβ internalized in an exosome-mediated manner was delivered to lysosomes to be degraded via the endocytic pathway within microglia.

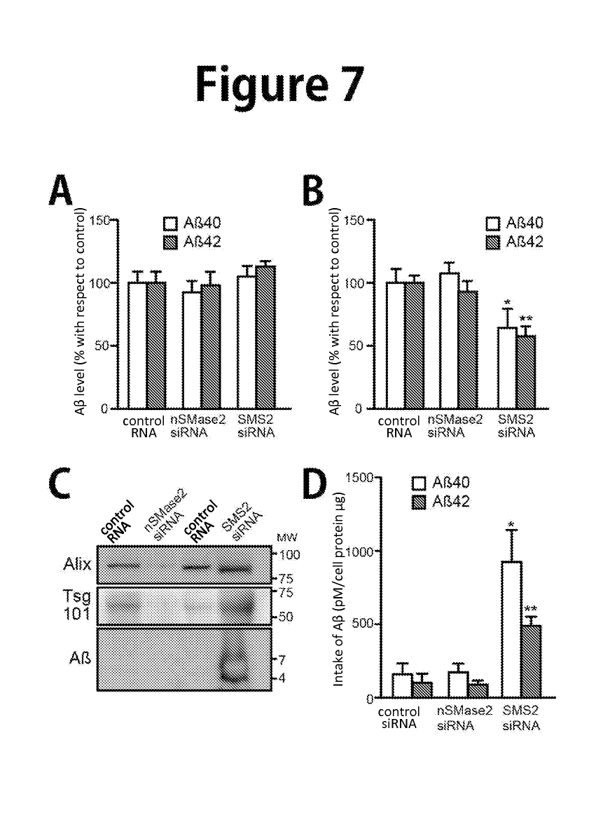

### (Experimental Results Related to FIG. 7)

### (Does Up-Regulation of Exosome Secretion Affect AP Clearance?)

Finally, the inventors examined whether modulation of exosome secretion can affect Aβ clearance by microglia. The inventors plated N2a cells on inserts for a Transwell setting and treated with siRNA for SMase2 or SMS2 to modulate the amount of exosomes released from the cells. The cells were concurrently transfected with an amyloid precursor protein (APP) gene to overexpress Aβ. 24 hours after the transfection, the inventors set the inserts onto 24-well multiplates, on which BV-2 cells had been seeded. Under this experimental setting, the inventors considered exosomes and Aβ, secreted from N2a cells, as capable of interacting with the BV-2 cells through medium shared among the cells. 24 hours after challenging the BV-2 cells with N2a-APP cells, Aβ levels in the medium were determined. Several studies have reported that sphingolipid metabolism is involved in APP processing for generating Aβ (Haughey N J et al., Biochimica et Biophysica Acta (BBA) (2010) 1801: 878-886). However, when there is no BV-2 cells on the lower wells, extracellular Aβ levels were unchanged even when N2a-APP cells were treated with siRNA of both nSMase2 or SMS2 (FIG. 7A). In contrast, in the presence of BV-2 cells in the lower wells, the levels of both Aβ40 and Aβ42 in the culture media were significantly decreased by SMS2 siRNA treatment (FIG. 7B). The inventors next analyzed levels of Alix, Tsg101, and Aβ in 100,000×g pellets collected from media of the N2a-APP cells, transfected with nSMase2 or SMS2 siRNA by Western blotting (FIG. 7C). In agreement with the previous data (FIGS. 3 C and 3D), the levels of released exosomes, estimated by the amounts of Alix and Tsg101, were obviously modulated by nSMase2 or SMS2 knockdown. In addition, Aβ was detected only in pellets from SMS2 treated N2a cultures. These findings suggest that acceleration of exosome secretion can promote formation of Aβ bound to exosomes, and the Aβ bound to exosomes can be in a state that is suitable for uptake by microglia. Indeed, the Aβ levels in the BV-2 cells were significantly increased in cultures treated with SMS2 siRNA compared with treatment with control RNA (FIG. 7D).

### (Discussion)

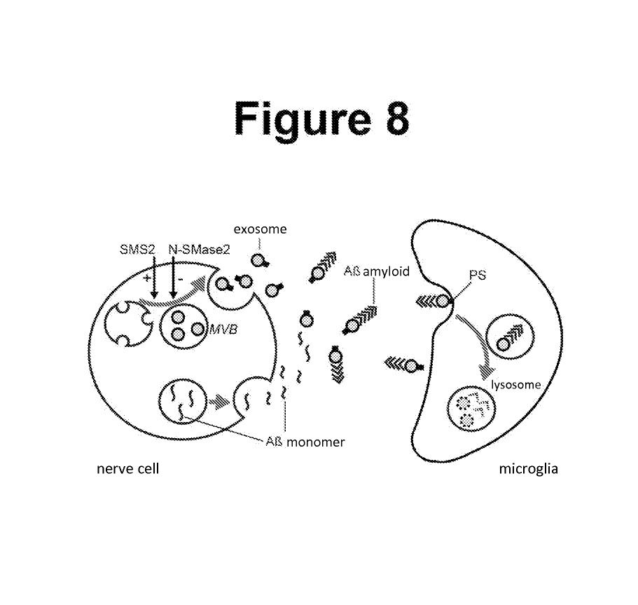

The inventors found that exosomes are constitutively secreted by nerve cells, and the exosomes dramatically promote Aβ amyloidogenesis on the surface thereof. Moreover, the aggregated Aβ bound to exosomes was further taken up by microglia for degradation. The inventors also demonstrated that up-regulation of exosome secretion, which was induced by modulation of sphingolipid metabolism, efficiently reduced extracellular levels of Aβ in a co-culture of nerve cells and microglial cells. In CNS, nerve cells are surrounded and surveyed by microglia to remove damaged structures such as apoptotic cells and obsolete synaptic connections (Kreutzberg G W, Trends Neurosci (1996) 19: 312-318). The present specification provides new insight into the coordinating mechanism between nerve cells and neighboring microglia for Aβ clearance using exosomes (see FIG. 8).

Regarding the formation of ordered Aβ aggregates, the seeding polymerization theory had been proposed (Harper J D et al., Annu Rev Biochem (1997) 66: 385-407). The transition of Aβ from a monomeric form to a polymer thereof requires a conformational change of the monomer Aβ induced by condensation or interaction with other specific molecules to function as a seed (Esler W P et al., Biochemistry (2000) 39: 6288-6295). The inventors have found herein that nerve cell-derived exosomes accelerate Aβ amyloidogenesis from monomeric Aβ (FIGS. 1C and 1D). In addition, it is suggested that seed Aβ can be produced by the binding of the monomer Aβ to exosome surface. Several molecular factors have been reported to accelerate Aβ polymerization, such as ApoE (Kim J et al., Neuron (2009) 63: 287-303), metallic ions including Zn²⁺ (Bush A I et al., Science (1994) 265: 1464-1467), heparan sulfate proteoglycan (HSPG) (Snow A D et al., Neuron (1994) 12: 219-234; Castillo G M et al., J Neurochem (1997) 69: 2452-2465), and various gangliosides (Yanagisawa K et al., Nat Med (1995) 1: 1062-1066; Ariga T et al., J Lipid Res (2008) 49: 1157-1175). Among such molecular factors, gangliosides, particularly GM1, are considered as one of the promising candidates for promoting Aβ amyloid production on exosomes. Accumulated evidence indicates that the specific structure of Aβ bound to GM1 serves as a template for forming Aβ aggregates (Matsuzaki K et al., Biochimica et Biophysica Acta (BBA)—Molecular and Cell Biology of Lipids (2010) 1801: 868-877). In addition, AP-bound GM1 has been found in AD brains exhibiting early pathological changes (Yanagisawa K et al., Nat Med (1995) 1: 1062-1066). It was demonstrated herein that stains on exosomes from fluorescently-labelled CTB expressed GM1 on the outer leaflet of exosome membrane (FIG. 4B) and the amount of GM1 in a 100,000×g pellet clearly correlated with the state of Aβ fibrillogenesis (FIGS. 3B, 3D, 3F, and 3G). However, the inventors are currently unable to eliminate the possibility of other molecules including ApoE, HSPG or unknown molecule being involved in Aβ fibrillogenesis. It was recently reported that Aβ fibrils formed on GM1-containing membranes exhibited significant toxicity toward PC12 cells (Okada T et al., J Mol Biol (2008) 382: 1066-1074). However, herein, addition of exosomes and Aβ to primary cultured cortical cultures significantly suppressed neuronal toxicity and the neuronal toxicity was inversely correlated with the amount of oligomeric Aβ (FIG. 2), but the neuronal toxicity was not inversely correlated with the amount of exosome-mediated Aβ fibrils (FIG. 1D). It is well known that Aβ fibrils exhibit polymorphism, which depends on differences in the first assembly process (Goldsbury C et al., J Mol Biol (2005) 352: 282-298; Petkova A T et al., Science (2005) 307: 262-265). Further careful examination is needed for the inventors to identify the mechanism behind exosome-mediated AP fibrillogenesis.

In the present specification, the inventors collected released exosomes and assessed the ability thereof to promote Aβ amyloidogenesis in TBS (FIGS. 1C and 1D) or in a culture medium (data not shown). The results suggest that the exosomes would be able to effectively promote the formation of Aβ fibrils in extracellular space. However, β-site cleavage of APP has recently been reported to occur in MVBs (Sharples R A et al., (2008) FASEB J 22: 1469-1478). In addition, Aβ42 has been found to preferentially localize to MVBs in normal murine and human brains. Furthermore, in a murine model of AD and in human AD brain, Aβ42 within nerve cells gradually accumulates with age (Takahashi R H et al., Am J Pathol (2002) 161: 1869-1879). Notably, GM1-bound Aβ, an amyloid seed, is preferentially observed in an endosome fraction of nerve cells from an aged monkey brain, while GM1-bound Aβ was not observed an endosome fraction of a young monkey brain (Kimura N et al., Neuroreport (2007) 18: 1669-1673). Thus, additional careful examinations at deeper degradation levels will be required to investigate the possibility that the intraluminal space of MVB provides another environment that can induce Aβ assembly prior to the release of exosomes and Aβ from cells. The inventors found herein that selective inhibition of nSMase2 activity reduced exosome secretion, while selective inhibition of SMS2 activity significantly increased exosome secretion (FIGS. 3C and 3D). nSMase2 is especially abundant in mammalian brains (Liu B et al., J Biol CChem (1998) 273: 34472-34479). This has two predicted transmembrane domains at the N terminus and is mainly localized in the plasma membrane (Karakashian A A et al., FASEB J (2004) 18: 968-970). SMS2 also has predicted six membrane-spanning regions, contributing to SM production at the plasma membrane (Huitema K et al., EMBO J. (2004) 23: 33-44). The inventors also found that exogenously added synthetic Cer and bacterial SMase increase exosome secretion (FIG. 3E). With this finding of the present invention, these findings suggest that elevation in local levels of Cer, especially at the plasma membranes, including endocytosed membrane regions, is important for the promotion of exosome generation. SMS1 not contributing as much as SMS2 does to exosome secretion is possibly due to localization of SMS1 which is responsible for the bulk of SM generation in Golgi apparatuses (Tafesse F G et al., J Biol CChem (2006) 281: 29421-29425). One possible mechanism with regard to a role for Cer in exosome generation would be Cer inducing a physical change in the endosomal membrane, which preferentially promotes the budding of intraluminal vesicles. Indeed, it has been reported that Cer induces a coalescence of small microdomains into larger microdomains to promote domain-induced budding of plasma membranes (Gulbins E et al., Oncogene (2003) 22: 7070-7077). Treatment with bacterial SMase induces intraluminal membrane budding from SM-containing synthetic giant liposomes (Trajkovic K et al., Science (2008) 319: 1244-1247). Alternatively, several pieces of evidence suggest that Cer can also affect endocytic transport. Cer production, induced by exocytosis of aSMase, reportedly promotes repair of plasma membranes and endocytosis (Tam C et al., J Cell Biol (2010) 189: 1027-1038). Further, exogenously added bacterial SMase is known to induce ATP-dependent endocytosis (Zha X et al., Cell Biol (1998) 140: 39-47). These findings suggest another possibility that Cer may promote exosome generation by changing the rate of endocytosis.

Previous studies have shown that microglia can directly take up Aβ itself and degrade it in late endosome-lysosomal compartments (Majumdar A et al., Mol Biol Cell (2007) 18: 1490-1496). Thus, it remains difficult to define what the functional significance of Aβ uptake together with exosomes is. One point that is conceivably important would be an increase in the efficiency of Aβ uptake into microglia. As demonstrated by the results from the inventors that Aβ rapidly forms amyloid fibrils in the presence of exosomes (FIGS. 1C and 1D), microglia can take up Aβ more rapidly after excessive production of Aβ, in the presence of exosomes. Furthermore, addition of exosomes indeed suppresses the formation of toxic oligomers (FIG. 2), but this is highly effective for avoiding impairment of nerve cells. Another conceivably important point that is postulated is a decrease in immunological reactions by the microglia. Accumulated evidence from in vitro and in vivo studies indicates that fibril Aβ promotes inflammatory responses in microglia, including the release of proinflammatory cytokines and reactive oxygen species (Cameron B et al., Neurobiol Dis (2010) 37: 503-509). Activatedmicroglia surrounding senile plaque, an excessive deposition of Aβ fibrils, contribute to chronic inflammation in the AD brain exhibiting a pathological change. In general, it is well understood that uptake of apoptotic bodies by microglia is associated with anti-inflammatory reactions (Magnus T et al., J Immunol (2001) 167: 5004-5010). In addition, it has recently been shown that oligodendrocyte-derived exosomes are taken up by microglia in an immunologically silent manner (Fitzner D et al., J Cell Sci (2011) 124: 447-458). This document also reports that exosome internalization was preferentially connected to inflammatory unresponsive microglia, which shows a low level of MHCII. In addition, the inventors found that mRNA expression of IL-1β and TNF-α did not change in BV-2 cells after N2a-derived exosome uptake (data not shown). Although further studies are needed, these data suggest that nerve cell-derived exosomes can remove Aβ by preventing proinflammatory reactions by microglia.

Other aggregate-prone proteins, including α-synuclein and prion protein, which cause the pathogenesis of Parkinson and Creutzfeldt-Jakob diseases, also bind to nerve cell exosomes (Fevrier B et al., Curr Opin Cell Biol (2004) 16: 415-421; Emmanouilidou E et al., J Neuroscience (2010) 30: 6838-6851). A challenging subject of studies in the future will be determining whether exosomes are involved in the assembly of these proteins and in clearance of these proteins through interaction with microglia. The inventors consider that when uptake/clearance activity of microglia decreases, secretion of exosomes having these proteins can cause the pathological events, which substantially occur in the extracellular space. Indeed, in the absence of exosome-removing cells, exosomes that bind to both normally and abnormally folded species of prion proteins are infectious, causing the spreading thereof between nerve cells (Fevrier B et al., Proc Natl Acad Sci USA (2004) 101: 9683-9688; Vella L J et al., J Pathol (2007) 211: 582-590). Furthermore, secreted a-synuclein bound to exosomes causes cell death of recipient nerve cells (Emmanouilidou E et al., J Neuroscience (2010) 30: 6838-6851). Aβ plaques can also be pathological structures constructed in a state lacking glial activity for removing exosomes. Indeed, a decrease in the number of microglia in a murine model of AD results in an increase of Aβ deposition (El Khoury J et al., Nat Med (2007) 13: 432-438).

Improvement of Aβ clearance is a potent strategy for AD therapy (Mawuenyega K G et al., Science (2010) 330: 1774). The present specification can provide a new approach using exosomes for Aβ removal. Modulation of exosome secretion by selective regulation of a Cer-metabolizing pathway is likely to be therapeutically useful. In addition, delivery technique of exosomes, including the targeting of intravenously injected exosomes into the brain, is currently being developed for therapeutic applications (Alvarez-Erviti L et al., Nat Biotechnol (2011) 29: 341-345). This can also be useful for exosome-mediated Aβ clearance in AD with some advantages, such as treatment with engineered exosomes or with required quantities of exosomes.

### Example 2

### Experiments with Other Sequences

Experiments similar to the aforementioned Example can be conducted using the following siRNA sequences.

The murine specific siRNA sequences that will be used are shown below.

(SEQ ID NO: 23)

| | |
|---|---|
| SMS2-i1 | 5′-ggucacuuggaaagucaaa-3′ (sense strand) |

antisense strand which is a complementary sequence thereof (SEQ ID NO: 24)

(SEQ ID NO: 25)

| | |
|---|---|
| SMS2-i2 | 5′-ccggacuacauccagauuu-3′ (sense strand) |

antisense strand which is a complementary sequence thereof (SEQ ID NO: 26)

(SEQ ID NO: 19)

| | |
|---|---|
| SMS2-i3 | 5′-ggaugguauugguuggguu-3′ (sense strand) |

antisense strand which is a complementary sequence thereof (SEQ ID NO: 20)

(SEQ ID NO: 94)

| | |
|---|---|
| SMS2-i4 | 5′-gcagauuguuguugaucau-3′ (sense strand) |

antisense strand which is a complementary sequence thereof (SEQ ID NO: 95)

(SEQ ID NO: 21)

| | |
|---|---|
| SMS2-i11 | 5′-ggcucuuucugcguuacaa-3′ (sense strand) |

antisense strand which is a complementary sequence thereof (SEQ ID NO: 22)

The siRNA sequences used, which are homologous between mice and humans, are shown below.

(SEQ ID NO: 27)

| | |
|---|---|
| SMS2-i5 | 5′-cauagagacagcaaaacuu-3′ (sense strand) |

antisense strand which is a complementary sequence thereof (SEQ ID NO: 28).

(SEQ ID NO: 1)

| | |
|---|---|
| SMS2-i6 | 5′-gcauuuucuguaucagaaa-3′ (sense strand) |

antisense strand which is a complementary sequence thereof (SEQ ID NO: 2)

(SEQ ID NO: 3)

| | |
|---|---|
| SMS2-i7 | 5′-gucacuucuggugguauca-3′ (sense strand) |

antisense strand which is a complementary sequence thereof (SEQ ID NO: 4)

(SEQ ID NO: 5)

| | |
|---|---|
| SMS2-i8 | 5′-cuguuuuggugguaccauu-3′ (sense strand) |

antisense strand which is a complementary sequence thereof (SEQ ID NO: 6)

The human specific siRNA sequences that were used are shown.

(SEQ ID NO: 7)

| | |
|---|---|
| SMS2-i104 | 5′-gggcauugccuucauauau-3′ (sense strand) |

antisense strand which is a complementary sequence thereof (SEQ ID NO: 8)

(SEQ ID NO: 9)

| | |
|---|---|
| SMS2-i105 | 5′-ggcuguuucugagauacaa-3′ (sense strand) |

antisense strand which is a complementary sequence thereof (SEQ ID NO: 10)

(SEQ ID NO: 11)

| | |
|---|---|
| SMS2-i106 | 5′-ggugguggauuguccauaa-3′ (sense strand) |

antisense strand which is a complementary sequence thereof (SEQ ID NO: 12)

(SEQ ID NO: 13)

| | |
|---|---|
| SMS2-i107 | 5′-ggauuguccauaacuggau-3′ (sense strand) |

antisense strand which is a complementary sequence thereof (SEQ ID NO: 14)

(SEQ ID NO: 15)

| | |
|---|---|
| SMS2-i108 | 5′-ccauaacuggaucacauau-3′ (sense strand) |

antisense strand which is a complementary sequence thereof (SEQ ID NO: 16)

(SEQ ID NO: 17)

| | |
|---|---|
| SMS2-i109 | 5′-gcacacgaacacuacacua-3′ (sense strand) |

antisense strand which is a complementary sequence thereof (SEQ ID NO: 18)

SiRNA (CTR-i) sequence that was used as the control is shown.

| SEQ ID NO: 96) | |
|---|---|
| CTR-i | 5′-uucuccgaacgugucacgu-3′ (sense strand) |

antisense strand (SEQ ID NO: 97).

### Example 3

### Experiments with nSMase 2

Experiments similar to the aforementioned Examples will be conducted using nSMase or an expression vector thereof.

It is possible to confirm the effect of nSMase 2 on diseases associated with amyloid β with the method described in Example 1 by transfecting an nSMase 2 expression vector (e.g., including the sequence described in SEQ ID NO: 83 or 85) with the same method as the method of introducing siRNA or the like while referring to known methods in the art.

### Example 4

### Example of Nucleic Acids Other than SiRNA (Ribozyme)

Next, a ribozymal sequence is designed based on the nucleic acid sequence of SMS2 described in SEQ ID NO: 87 or 88 by using the method such as that described in (Kikuchi, Y. & Sasaki, N., Nucl Acids Res, 1991, 19, 6751., Yo KIKUCHI, Kagaku to Seibutsu (Chemistry and Biology), 1992, 30, 112.)

With respect to the above matter, the effect of ribozymes of the present invention can be confirmed by the method using a mouse and a nerve cell described in the aforementioned

### Examples

### Example 5

### Screening for Antisense Nucleic Acid for SMS2

The present Example demonstrated the effectiveness of an antisense nucleic acid that had been designed based on a homologous domain (consecutive homologous domains which are tridecamer or more) in the base sequences of SMS2 of a human and mouse.

Antisense oligonucleotides are designed and manufactured, and knockdown experiments are conducted with human HIK293 cells. Efficient gene silencing by delivery of locked nucleic acid antisense oligonucleotides, unassisted by transfection reagents in Nucleic Acid Research 2010, Vol 38, No. 1 was referred for the design of sequence structure.

The sequence and shape of tridecamer antisense oligonucleotide that was used in the Examples are shown below. The nucleic acid that was used is an LNA-containing nucleic acid, also called LNA Gapmer antisense oligonucleotide, is an LNA (Locked nucleic acid) in capital letters but is DNA in lower case letters. LNA is a type of BNA (Bridged Nucleic Acid). More than 10 types of BNAs are known. Among them, LNAs are engineered nucleic acids in which the 2′ position and 4′ position of sugar are cross-linked with —O—CH₂— to fix the conformation to an N-type. LNAs are available from Funakoshi and the like. All are linked by a phosphorothioate-modified backbone. All Cs in the LNA moiety are methylcytosine. In the following example, three Locked Nucleic Acids (LNA) are comprised in the 5′ terminus and two LNAs are comprised in the 3′ terminus.

| | | Antisense | |
|---|---|---|---|
| | | nucleic acid | |
| | | sequence | |
| | Name | (5′-3′) | |
| 1 | SMS2-13-003 → | TGAtaccaccaGA | (SEQ ID NO: 29) |
| 2 | SMS2-13-006 → | TGCagatgatcCC | (SEQ ID NO: 30) |
| 3 | SMS2-13-007 → | CGTgttgtgatAT | (SEQ ID NO: 31) |
| 4 | SMS2-13-008 → | ACTtgtotgggAG | (SEQ ID NO: 32) |
| 5 | SMS2-13-009 → | AGAggaagtctCC | (SEQ ID NO: 33) |
| 6 | SMS2-13-010 → | AGAtggggaacCA | (SEQ ID NO: 34) |
| 7 | SMS2-13-011 → | AGTctccattgAG | (SEQ ID NO: 35) |
| 8 | SMS2-13-012 → | CCAgaagtgacGA | (SEQ ID NO: 36) |
| 9 | SMS2-13-014 → | TTGcctgagagTC | (SEQ ID NO: 37) |
| 10 | SMS2-13-017 → | AAGttttgctgTC | (SEQ ID NO: 38) |
| 11 | SMS2-13-019 → | TTGaagcagccAG | (SEQ ID NO: 39) |
| 12 | SMS2-13-020 → | GCAgcaaggaaTT | (SEQ ID NO: 40) |

Knock down experiments were conducted with human HEK293 cells by using 12 types of LNA-Gapmer antisense oligonucleotides that were made above. The LNA Gapmer antisense oligonucleotides were directly added to cell culture solutions at a final concentration of 5 μM. Quantitative PCR was performed 72 hours after transformation. G3PDH was used as an endogenous control.

To measure the expression amount of human SMS2, the following primer sequences were used:

| | | |
|---|---|---|
| Fw primer: | TCAATGGAGACTCTCAGGC | (SEQ ID NO: 90) |
| and | | |
| Rv primer: | CCGCTGAAGAGGAAGTCTC. | (SEQ ID NO: 91) |

To measure the expression amount of human G3PDH, the following primer sequences were used:

| | | |
|---|---|---|
| Fw primer: | CCTTCCGTGTCCCCACTG | (SEQ ID NO: 92) |
| and | | |
| Rv primer: | ACCCTGTTGCTGTAGCCAA. | (SEQ ID NO: 93) |

As a result thereof, SMS2 gene expression suppression can be confirmed in each of the Examples in comparison to saline administered cells (antisense-free cell). In addition, SMS2 gene expression suppression of 50% or greater in comparison to saline administered cells (antisense-free cell) was able to be confirmed in SMS2-13-006, 007, 008, 012, 014, 017, and 019 <SEQ ID NO: 30, 31, 32, 36, 37, 38, and 39, respectively>.

### Example 6

### Experiments with Antisense

Experiments similar to Example 1 or 2 are conducted by using the antisense sequences of SMS2 that were identified in Example 5.

In the present Example, it is possible to confirm the effect of nSMase 2 on diseases associated with amyloid β with the method described in Example 1 by transfecting these antisense sequences (SEQ ID NOs: 29-40) with the same method as the method of introducing siRNA or the like while referring to known methods in the art.

As described above, the present invention is exemplified by the use of its preferred Embodiments. However, it is understood that the scope of the present invention should be interpreted solely based on the claims. It is also understood that any patent, any patent application, and any references cited in the present specification should be incorporated by reference in the present specification in the same manner as the contents are specifically described therein.

### INDUSTRIAL APPLICABILITY

The present invention provides an agent for the treatment or prevention for a condition, symptom or disease associated with amyloid β.

### [Sequence Listing Free Text]

SEQ ID NO: 1: a sequence of a sense strand portion of a duplex portion of SMS2-i6
SEQ ID NO: 2: a sequence of an antisense strand of a duplex portion of SMS2-i6
SEQ ID NO: 3: a sequence of a sense strand portion of a duplex portion of SMS2-i7
SEQ ID NO: 4: a sequence of an antisense strand of a duplex portion of SMS2-i7
SEQ ID NO: 5: a sequence of a sense strand portion of a duplex portion of SMS2-i8
SEQ ID NO: 6: a sequence of an antisense strand of a duplex portion of SMS2-i8
SEQ ID NO: 7: a sequence of a sense strand portion of a duplex portion of SMS2-i104
SEQ ID NO: 8: a sequence of an antisense strand of a duplex portion of SMS2-i104
SEQ ID NO: 9: a sequence of a sense strand of a duplex portion of SMS2-i105
SEQ ID NO: 10: a sequence of an antisense strand of a duplex portion of SMS2-i105
SEQ ID NO: 11: a sequence of a sense strand of a duplex portion of SMS2-i106
SEQ ID NO: 12: a sequence of an antisense strand of a duplex portion of SMS2-i106
SEQ ID NO: 13: a sequence of a sense strand of a duplex portion of SMS2-i107
SEQ ID NO: 14: a sequence of an antisense strand of a duplex portion of SMS2-i107
SEQ ID NO: 15: a sequence of a sense strand of a duplex portion of SMS2-i108
SEQ ID NO: 16: a sequence of an antisense strand of a duplex portion of SMS2-i108
SEQ ID NO: 17: a sequence of a sense strand of a duplex portion of SMS2-i109
SEQ ID NO: 18: a sequence of an antisense strand of a duplex portion of SMS2-i109
SEQ ID NO: 19: a sequence of a sense strand of a duplex portion of SMS2-i3
SEQ ID NO: 20: a sequence of an antisense strand of a duplex portion of SMS2-i3
SEQ ID NO: 21: a sequence of a sense strand of a duplex portion of SMS2-i11
SEQ ID NO: 22: a sequence of an antisense strand of a duplex portion of SMS2-i11
SEQ ID NO: 23: a sequence of a sense strand portion of a duplex portion of SMS2-i1
SEQ ID NO: 24: a sequence of an antisense strand of a duplex portion of SMS2-i1
SEQ ID NO: 25: a sequence of a sense strand portion of a duplex portion of SMS2-i2
SEQ ID NO: 26: a sequence of an antisense strand of a duplex portion of SMS2-i2
SEQ ID NO: 27: a sequence of a sense strand of a duplex portion of SMS2-i5
SEQ ID NO: 28: a sequence of an antisense strand of a duplex portion of SMS2-i5
SEQ ID NO: 29: a sequence of an antisense nucleic acid of SMS2-13-003
SEQ ID NO: 30: a sequence of an antisense nucleic acid of SMS2-13-006
SEQ ID NO: 31: a sequence of an antisense nucleic acid of SMS2-13-007
SEQ ID NO: 32: a sequence of an antisense nucleic acid of SMS2-13-008
SEQ ID NO: 33: a sequence of an antisense nucleic acid of SMS2-13-009
SEQ ID NO: 34: a sequence of an antisense nucleic acid of SMS2-13-010
SEQ ID NO: 35: a sequence of an antisense nucleic acid of SMS2-13-011
SEQ ID NO: 36: a sequence of an antisense nucleic acid of SMS2-13-012
SEQ ID NO: 37: a sequence of an antisense nucleic acid of SMS2-13-014
SEQ ID NO: 38: a sequence of an antisense nucleic acid of SMS2-13-017
SEQ ID NO: 39: a sequence of an antisense nucleic acid of SMS2-13-019
SEQ ID NO: 40: a sequence of an antisense nucleic acid of SMS2-13-020
SEQ ID NO: 41: a sense strand siRNA sequence of a duplex portion with regard to SMS1; 5′-AUACAUUGUAAUACACCGAUACAGG-3′
SEQ ID NO: 42: an antisense strand siRNA sequence of a duplex portion with regard to SMS1; 5′-CCUGUAUCGGUGUAUUACAAUGUAU-3′
SEQ ID NO: 43: a sense strand siRNA sequence of a duplex portion with regard to SMS2; 5′-AUACAUAGUUAUACAGCGAUACAGG-3′
SEQ ID NO: 44: an antisense strand siRNA sequence of a duplex portion with regard to SMS2; 5′-CCUGUAUCGCUGUAUAACUAUGUAU-3′
SEQ ID NO: 45: a sense strand siRNA sequence of a duplex portion with regard to aSMase; 5′-AUUGGUUUCCCUUUAUGAAGGGAGG-3′
SEQ ID NO: 46: an antisense strand siRNA sequence of a duplex portion with regard to aSMase; 5′-CCUCCCUUCAUAAAGGGAAACCAAU-3′
SEQ ID NO: 47: a sense strand siRNA sequence of a duplex portion with regard to nSMase1; 5′-AAUAGAACCACAUCUGCAUUCUUGG-3′
SEQ ID NO: 48: an antisense strand siRNA sequence of a duplex portion with regard to nSMase1; 5′-CCAAGAAUGCAGAUGUGGUUCUAUU-3′
SEQ ID NO: 49: a sense strand siRNA sequence of a duplex portion with regard to nSMase2; 5′-AAUCGAUGUAGAUCUUGAUCUGAGG-3′
SEQ ID NO: 50: an antisense strand siRNA sequence of a duplex portion with regard to nSMase2; 5′-CCUCAGAUCAAGAUCUACAUCGAUU-3′
SEQ ID NO: 51: a human amyloid precursor protein (APP) 770 amplifying primer; 5′-ATGCTGCCCGGTTTGG-3′
SEQ ID NO: 52: a human amyloid precursor protein (APP) 770 amplifying antisense primer; 5′-CTAGTTCTGCATCTGCTCAAAGAACTTG-3′
SEQ ID NO: 53: a sequence of a sense strand portion of SMS2-i1
SEQ ID NO: 54: a sequence of an antisense strand of SMS2-i1
SEQ ID NO: 55: a sequence of a sense strand portion of SMS2-i2
SEQ ID NO: 56: a sequence of an antisense strand of SMS2-i2
SEQ ID NO: 57: a sequence of a sense strand of SMS2-i3
SEQ ID NO: 58: a sequence of an antisense strand of SMS2-i3
SEQ ID NO: 59: a sequence of a sense strand of SMS2-i4
SEQ ID NO: 60: a sequence of an antisense strand of SMS2-i4
SEQ ID NO: 61: a sequence of a sense strand of SMS2-i5
SEQ ID NO: 62: a sequence of an antisense strand of SMS2-i5
SEQ ID NO: 63: a sequence of a sense strand portion of SMS2-i6
SEQ ID NO: 64: a sequence of an antisense strand of SMS2-i6
SEQ ID NO: 65: a sequence of a sense strand portion of SMS2-i7
SEQ ID NO: 66: a sequence of an antisense strand of SMS2-i7
SEQ ID NO: 67: a sequence of a sense strand portion of SMS2-i8
SEQ ID NO: 68: a sequence of an antisense strand of SMS2-i8
SEQ ID NO: 69: a sequence of a sense strand of SMS2-i11
SEQ ID NO: 70: a sequence of an antisense strand of SMS2-i11
SEQ ID NO: 71: a sequence of a sense strand portion of SMS2-i104
SEQ ID NO: 72: a sequence of an antisense strand of SMS2-i104
SEQ ID NO: 73: a sequence of a sense strand of SMS2-i105
SEQ ID NO: 74: a sequence of an antisense strand of SMS2-i105
SEQ ID NO: 75: a sequence of a sense strand of SMS2-i106
SEQ ID NO: 76: a sequence of an antisense strand of SMS2-i106
SEQ ID NO: 77: a sequence of a sense strand of SMS2-i107
SEQ ID NO: 78: a sequence of an antisense strand of SMS2-i107
SEQ ID NO: 79: a sequence of a sense strand of SMS2-i108
SEQ ID NO: 80: a sequence of an antisense strand of SMS2-i108
SEQ ID NO: 81: a sequence of a sense strand of SMS2-i109
SEQ ID NO: 82: a sequence of an antisense strand of SMS2-i109
SEQ ID NO: 83: a nucleic acid sequence of human N-SMase2 <NM_{—}018667>
SEQ ID NO: 84; an amino acid sequence of human N-SMase2
SEQ ID NO: 85: a nucleic acid sequence of murine N-SMase2 <NM_{—}021491>
SEQ ID NO: 86: an amino acid sequence of murine N-SMase2
SEQ ID NO: 87: a nucleic acid sequence of human SMS2
SEQ ID NO: 88: a nucleic acid sequence of murine SMS2
SEQ ID NO: 89: amyloid β (1-55)=DAEFRHDSGY EVHHQKLVFF AEDVGSNKGA IIGLMVGGVV IATVIVITLVMLKKK
SEQ ID NO: 90: a primer sequence used for assaying an expression amount of human SMS2: Fw primer: TCAATGGAGACTCTCAGGC
SEQ ID NO: 91: a primer sequence used for assaying an expression amount of human SMS2:Rv primer:CCGCTGAAGAGGAAGTCTC
SEQ ID NO: 92: a primer sequence used for assaying an expression amount of human G3PD:Fw primer:CCTTCCGTGTCCCCACTG
SEQ ID NO: 93: a primer sequence used for assaying an expression amount of human G3PDH:Rv primer:ACCCTGTTGCTGTAGCCAA
SEQ ID NO: 94: a sense strand of a duplex portion of SMS2-i4=5′-gcagauuguuguugaucau-3′ (sense strand)
SEQ ID NO: 95: an antisense strand of a duplex portion of SMS2-i4
SEQ ID NO: 96: an siRNA (CTR-i) sequence used as a control: CTR-i 5′-uucuccgaacqugucacgu-3′ (sense strand)
SEQ ID NO: 97: same antisense strand
SEQ ID NO: 98: SEQ ID NO: 43+dTdT
SEQ ID NO: 99: dTdT+SEQ ID NO: 44

1. A method for screening a treatment substance or prevention substance for a disease associated with amyloid β, comprising:
(1) allowing protein of neutral sphingomyelinase 2 (N-SMase2) and/or sphingomyelin synthetic enzyme 2 (SMS2) to contact with a test substance;
(2) comparing enzyme activity of the protein of the N-SMase2 and/or SMS2 to which the test substance has been contacted, with enzyme activity of protein of the N-SMase2 and/or SMS2 to which the test substance has not been contacted; and
(3) when the enzyme activity of the protein of the N-SMase2 to which the test substance has been contacted is increased compared to the enzyme activity of the protein of the N-SMase2 to which the test substance has not been contacted, and/or the enzyme activity of the protein of the SMS2 to which the test substance has been contacted is decreased compared to the enzyme activity of the protein of the SMS2 to which the test substance has not been contacted, selecting the test substance as a treatment substance or prevention substance of the disease associated with amyloid β.

2. A method for screening a treatment substance or prevention substance for a disease associated with amyloid β, comprising:
(1) allowing a cell to contact with a test substance;
(2) comparing expression of N-SMase2 and/or SMS2 in the cell to which the test substance has been contacted, with expression of N-SMase2 and/or SMS2 in a control cell to which the test substance has not been contacted; and
(3) when the expression of the N-SMase2 in the cell to which the test substance has been contacted is increased compared to the expression of the N-SMase2 in the control cell to which the test substance has not been contacted, and/or when the expression of SMS2 in the cell to which the test substance has been contacted is decreased compared to the expression of SMS2 in the cell to which the test substance has not been contacted, selecting the test substance as a treatment substance or prevention substance of the disease associated with amyloid β.

3. A method for screening a treatment substance or prevention substance for a disease associated with amyloid β, comprising:
(1) allowing a cell to contact with a test substance;
(2) comparing an exosome secretion level in the cell to which the test substance has been contacted, with an exosome secretion level in a control cell to which the test substance has not been contacted; and
(3) when an exosome secretion level in the cell to which the test substance has been contacted is increased compared to an exosome secretion level in the control cell to which the test substance has not been contacted, selecting the test substance as a treatment substance or prevention substance of the disease associated with amyloid β.

4. The method according to claim 2 or 3, wherein said cell and said control cell are a nerve cell.

5. A pharmaceutical composition for treating or preventing a disease associated with amyloid β, comprising a substance for increasing enzyme activity or expression of protein of N-SMase2.

6. A pharmaceutical composition for treating or preventing a disease associated with amyloid β, comprising N-SMase2.

7. A pharmaceutical composition for treating or preventing a disease associated with amyloid β, comprising a substance for suppressing enzyme activity or expression of protein of SMS2.

8. The pharmaceutical composition according to claim 7, wherein said substance is a nucleic acid.

9. The pharmaceutical composition according to claim 8, wherein said nucleic acid is an siRNA and/or antisense nucleic acid.

10. The pharmaceutical composition according to claim 9, wherein said siRNA consists of any one or more selected from the group consisting of siRNAs in the following (a) to (p):
(a) an siRNA wherein one strand of the duplex RNA portion is a base sequence set forth by SEQ ID NO: 1 and the other strand of the duplex RNA portion is a base sequence set forth by SEQ ID NO: 2;
(b) an siRNA wherein one strand of the duplex RNA portion is a base sequence set forth by SEQ ID NO: 3 and the other strand of the duplex RNA portion is a base sequence set forth by SEQ ID NO: 4;
(c) an siRNA wherein one strand of the duplex RNA portion is a base sequence set forth by SEQ ID NO: 5 and the other strand of the duplex RNA portion is a base sequence set forth by SEQ ID NO: 6;
(d) an siRNA wherein one strand of the duplex RNA portion is a base sequence set forth by SEQ ID NO: 7 and the other strand of the duplex RNA portion is a base sequence set forth by SEQ ID NO: 8;
(e) an siRNA wherein one strand of the duplex RNA portion is a base sequence set forth by SEQ ID NO: 9 and the other strand of the duplex RNA portion is a base sequence set forth by SEQ ID NO: 10;
(f) an siRNA wherein one strand of the duplex RNA portion is a base sequence set forth by SEQ ID NO: 11 and the other strand of the duplex RNA portion is a base sequence set forth by SEQ ID NO: 12;
(g) an siRNA wherein one strand of the duplex RNA portion is a base sequence set forth by SEQ ID NO: 13 and the other strand of the duplex RNA portion is a base sequence set forth by SEQ ID NO: 14;
(h) an siRNA wherein one strand of the duplex RNA portion is a base sequence set forth by SEQ ID NO: 15 and the other strand of the duplex RNA portion is a base sequence set forth by SEQ ID NO: 16;
(i) an siRNA wherein one strand of the duplex RNA portion is a base sequence set forth by SEQ ID NO: 17 and the other strand of the duplex RNA portion is a base sequence set forth by SEQ ID NO: 18;
(j) an siRNA wherein one strand of the duplex RNA portion is a base sequence set forth by SEQ ID NO: 19 and the other strand of the duplex RNA portion is a base sequence set forth by SEQ ID NO: 20;
(k) an siRNA wherein one strand of the duplex RNA portion is a base sequence set forth by SEQ ID NO: 21 and the other strand of the duplex RNA portion is a base sequence set forth by SEQ ID NO: 22;
(l) an siRNA wherein one strand of the duplex RNA portion is a base sequence set forth by SEQ ID NO: 23 and the other strand of the duplex RNA portion is a base sequence set forth by SEQ ID NO: 24, which is a complementary sequence thereof;
(m) an siRNA wherein one strand of the duplex RNA portion is a base sequence set forth by SEQ ID NO: 25 and the other strand of the duplex RNA portion is a base sequence set forth by SEQ ID NO: 26, which is a complementary sequence thereof;
(n) an siRNA wherein one strand of the duplex RNA portion is a base sequence set forth by SEQ ID NO: 27 and the other strand of the duplex RNA portion is a base sequence set forth by SEQ ID NO: 28, which is a complementary sequence thereof;
(o) an siRNA wherein one strand of the duplex RNA portion is a base sequence set forth by SEQ ID NO: 43 and the other strand of the duplex RNA portion is a base sequence set forth by SEQ ID NO: 44;
(p) an siRNA according to any of (a) to (o), wherein one to several nucleotides are added, inserted, deleted or substituted in one or both of the base sequences, and having an activity of suppressing the expression of SMS2.
